(19) United States
(12) Patent Application Publication
(10) Pub. No.: US 2024/0277839 A1
(43) Pub. Date: Aug. 22, 2024

(54) HIGH CONCENTRATION FORMULATION AND USES THEREOF

(71) Applicant: CSL Innovation Pty Ltd., Melborne VIC (AU)

(72) Inventors: Michael JOHNSTON, Williamstown, Victoria (AU); Dianna Grace GOODALL, Melbourne, Victoria (AU); Nathan Aaron EDWARDS, Pascoe Vale South, Victoria (AU)

(21) Appl. No.: 18/004,004

(22) PCT Filed: Jul. 5, 2021
(86) PCT No.: PCT/AU2021/050714

§ 371 Date: Dec. 30, 2022

(30) Foreign Application Priority Data
Jul. 3, 2020 (EP) 20184004.8

(51) Int. Cl. A61K 39/395; A61K 39/00; A61K 47/22; A61K 47/26; A61P 7/02; C07K 16/36

(52) U.S. Cl. CPC A61K 39/39591; A61K 47/22; A61K 47/26; A61P 7/02; C07K 16/36; A61K 2039/505; C07K 2317/76

(57) ABSTRACT

The present disclosure relates to high concentration protein formulations and uses thereof. In particular, it is based on the identification of a pharmaceutical formulation for a protein comprising an antigen binding domain that binds to or specifically binds to Factor XII and/or FXIIa.

### FIELD

The present disclosure relates to high concentration protein formulations and uses thereof.

### BACKGROUND

Normal blood coagulation is a highly conserved process in mammalian biology involving complex physiological and biochemical processes comprising activation of a coagulation factor (or clotting factor) cascade ultimately leading to fibrin formation and platelet aggregation. The blood coagulation cascade comprises an “extrinsic” pathway, the primary means of coagulation initiation, and an “intrinsic” pathway, which contributes to stabilisation of the fibrin clot.

The majority of coagulation factors involved in the coagulation cascade are precursors of proteolytic enzymes known as zymogens. These enzymes circulate in the blood in a non-activated form and only participate in the coagulation cascade once they become activated (e.g. by proteolytic cleavage).

Factor XII (FXII, Hageman factor) is an essential coagulation protein for initiation of the intrinsic coagulation cascade. Activation of FXII to produce activated FXII (FXIIa) leads to activation of Factor XI to Factor XIa and C1 esterases (C1r, C1s), the first components of the macromolecular complex of C1 and the classical complement pathway. Activation of FXI leads to a series of proteolytic reactions resulting in thrombin generation and the haemostatic pathway, whilst activation of the complement system leads to increased vascular permeability, chemotaxis of phagocytic cells, activation of inflammatory cells, opsonization of foreign particles, direct killing of cells and tissue damage.

Despite its role in activation of the intrinsic coagulation cascade, and activation of the classical complement pathway, deficiencies in FXII are not associated with bleeding abnormalities. However, dysregulation of these pathways can lead to serious conditions.

Although antibodies and inhibitors against FXII and/or FXIIa exist, there are an increasing number of challenges in formulation development for drug manufacturers. For example, an ideal inhibitor of FXII/FXIIa will not increase the risk of bleeding, be non-immunogenic and has to be administered as sparingly as possible. In addition, subcutaneous administration is fast becoming a preferred alternative to intravenous administration. Subcutaneous injection of a drug using a self-administered delivery device, such as an auto-injector, pen, or pre-filled syringe, is not only more convenient for the patient, but also reduces healthcare costs by minimising hospital visits.

However, there are numerous challenges associated with formulating high concentration antibody formulations (e.g., ≥100 mg/ml protein) suitable for subcutaneous administration. Formulations for subcutaneous administration typically require higher concentrations of product so as to achieve smaller injection volumes, yet increasing protein concentration often negatively impacts protein aggregation and degradation, solubility, stability, and viscosity. In addition to changes in intrinsic protein properties, manufacturing challenges also exist including, difficulties with processing and storage, changes in formulation composition and rheological and syringeability properties of the final formulation. For example, viscous solutions typically require a higher injection force to administer, therefore a prolonged injection time may also be required contributing to patient pain and discomfort.

Various solutions to manufacturing high concentration antibody formulations include lyophilised formulations for reconstitution, bufferless formulations and the addition of high concentrations of salt or other additives to reduce aggregation and/or the viscosity of the formulation. However, the use of excessive amounts of such excipients, may lead to hypertonic preparations or changes in ionic strength of the formulation and related protein aggregation issues.

Thus, there is a need for formulations comprising protein therapeutics, for example antibodies that target the coagulation pathway, that are stable long-term and free of aggregation at high antibody concentrations.

### SUMMARY

The present disclosure is based on the identification of a pharmaceutical formulation for a protein comprising an antigen binding domain that binds to or specifically binds to Factor XII and/or an activated form thereof (i.e., activated FXII; FXIIa).

In producing suitable formulations, the inventors found that they can produce pharmaceutical formulations, e.g., liquid pharmaceutical formulations, comprising high concentrations of a protein comprising an antigen binding domain that binds to or specifically binds to FXII and/or FXIIa (i.e., concentrations of at least about 100 mg/ml), which remained soluble and maintained a viscosity suitable for injection (e.g., by subcutaneous administration). The high concentration formulation of the present disclosure comprises an organic acid, a non-ionic surfactant, an amino acid stabiliser and optionally a polyol. Notably, in producing the formulation of the present disclosure the inventors found that additional salts and/or stabilising agents were not required. Furthermore, in increasing the protein concentration of the antibody in the formulation (e.g., from about 100 mg/ml to about 170 mg/ml), the inventors found that changes (i.e., increasing or decreasing) to the concentration of excipients was not always required.

The findings by the inventors provide the basis for a pharmaceutical formulation comprising at least about 100 mg/ml of a protein comprising an antigen binding domain that binds to or specifically binds to Factor XII and/or an activated form thereof, wherein the formulation has a dynamic (i.e., absolute) viscosity of less than about 30 mPa*s at 20° C. The findings by the inventors also provide the basis for methods of treating a condition or disorder, e.g., a thrombotic disorder, an inflammatory disorder and/or a thrombo-inflammatory disorder in a subject.

The present disclosure provides, a liquid pharmaceutical formulation comprising at least about 100 mg/ml of a protein comprising an antigen binding domain that binds to or specifically binds to Factor XII and/or an activated form thereof, an organic acid buffer, a non-ionic surfactant and an amino acid stabilizer, wherein the formulation has a pH of 5.0 to 6.5 and a dynamic viscosity of less than about 30 mPa*s at 20° C.

In one example, the protein of the present disclosure is present in the formulation at a concentration of at least 100 mg/ml. For example, the protein is present in the formulation at a concentration of between 100 mg/ml and 200 mg/ml. For example, the protein is present in the formulation at a concentration of about 100 mg/ml, or about 110 mg/ml, or about 120 mg/ml, or about 130 mg/ml, or about 140 mg/ml, or about 150 mg/ml, or about 160 mg/ml, or about 170 mg/ml, or about 180 mg/ml, or about 190 mg/ml, or about 200 mg/ml. In one example, the protein is present in the formulation at a concentration of about 100 mg/ml, or about 120 mg/ml, or about 150 mg/ml, or about 170 mg/ml. In one example, the protein is present in the formulation at a concentration of about 100 mg/ml. In one example, the protein is present in the formulation at a concentration of about 150 mg/ml. In one example, the protein is present in the formulation at a concentration of at least about 150 mg/ml. In one example, the protein is present in the formulation at a concentration of about 160 mg/ml to about 180 mg/ml. For example, the protein is present in the formulation at a concentration of about 170 mg/ml. In one example, the protein is present in the formulation at a concentration of greater than 200 mg/ml. For example, the protein is present in the formulation at a concentration of about 200 mg/ml, or about 210 mg/ml, or about 220 mg/ml.

In one example, the protein comprising an antigen binding domain that binds to or specifically binds to Factor XII and/or an activated form thereof binds to Factor XII or to activated Factor XII (Factor XIIa). In one example, the protein that binds to Factor XII/XIIa binds to or specifically binds to Factor XII. In one example, the protein that binds to Factor XII/XIIa binds to or specifically binds to activated Factor XII (Factor XIIa).

In one example, the protein binds to or specifically binds to Factor XII and antagonises activity of Factor XII and/or Factor XIIa. In one example, the protein binds to or specifically binds to Factor XII and antagonises activation of the Factor XII and/or Factor XIIa. Reference herein to a protein or antibody that “binds to” Factor XII provides literal support for a protein or antibody that “binds specifically to” Factor XII.

In one example, the protein binds to or specifically binds to activated Factor XII (Factor XIIa) and antagonises activity of Factor XII and/or Factor XIIa. In one example, the protein binds to or specifically binds to activated Factor XII and antagonises activation of the Factor XII and/or Factor XIIa. Reference herein to a protein or antibody that “binds to” activated Factor XIIa provides literal support for a protein or antibody that “binds specifically to” activated Factor XIIa.

In one example, the protein comprises an antigen binding domain of an antibody. For example, the protein comprises at least a heavy chain variable region (V_{H}) and a light chain variable region (V_{L}), wherein the V_{H} and V_{L} bind to form a Fv comprising an antigen binding domain.

In one example, the V_{H} and the V_{L} are in a single polypeptide chain. For example, the protein is:
(i) a single chain Fv fragment (scFv);
(ii) a dimeric scFv (di-scFv); or
(iii) at least one of (i) and/or (ii) linked to a constant region of an antibody, Fc or a heavy chain constant domain (C_{H}) C_{H}2 and/or C_{H}3.

In one example, the V_{L} and V_{H} are in separate polypeptide chains. For example, the protein is:
(i) a diabody;
(ii) a triabody;
(iii) a tetrabody;
(iv) a Fab;
(v) a F(ab′)₂;
(vi) a Fv; or
(vii) one of (i) to (vi) linked to a constant region of an antibody, Fc or C_{H}2 and/or C_{H}3.

The foregoing proteins (described in the previous two lists) can also be referred to as antigen binding domains of antibodies.

In one example, the protein is an antibody or antigen binding fragment thereof (e.g., a scFv comprising the variable regions of the antibody). Exemplary antibodies are described, for example, in WO 2013/014092 and WO 2017/173494 which are incorporated herein by reference.

In one example, the protein comprises a scFv. In one example, the protein comprises a scFv that binds to or specifically binds to Factor XII and/or Factor XIIa (and e.g., antagonizes activity of the Factor XII and/or Factor XIIa or antagonizes activation of the Factor XII and/or Factor XIIa).

In one example, the protein comprising an antigen binding domain that binds to Factor XII and/or Factor XIIa is an antibody, i.e., a full-length antibody. For example, the antibody is an anti-FXII antibody. In another example, the antibody is an anti-FXIIa antibody.

In one example, the protein is recombinant, chimeric, de-immunised, humanised, human or primatised. In one example, the protein or antibody is human. In one example, the protein or antibody is humanised.

In one example, the antibody is a monoclonal antibody.

In one example, the antibody is an IgG antibody. For example, the antibody is an IgG₁, or an IgG₂, or an IgG₃, or an IgG₄ antibody.

In one example, the antibody is an IgG₄ antibody.

In one example, the antibody is a monoclonal IgG₄ antibody.

In one example, the protein comprises an Fc region. For example, the Fc region is a human IgG₁ Fc region or a human IgG₄ Fc region or a stabilised human IgG₄ Fc region. For example, the Fc region is a human IgG₄ Fc region. In one example, the antibody Fc region is modified to prevent dimerisation, (e.g., as discussed herein).

In one example, the antibody or antigen binding fragment thereof comprises an IgG₄ constant region.

In one example, the IgG₄ constant region is a stabilised IgG₄ constant region. For example, the IgG₄ constant region comprises a stabilised hinge region. For example, the stabilised IgG₄ constant regions comprise a proline at position 241 of the hinge region according to the system of Kabat (Kabat et al., Sequences of Proteins of Immunological Interest Washington DC United States Department of Health and Human Services, 1987 and/or 1991).

In one example, the protein comprising an antigen binding domain that binds to or specifically binds to at least Factor XII and/or an activated form thereof. This does not mean that the protein of the present disclosure does not bind to other proteins, only that the protein (or part thereof) is specific to Factor XII and/or activated Factor XII and does not bind proteins in general. This term also does not exclude e.g., a bispecific antibody or protein comprising antigen binding domains thereof, which can specifically bind to Factor XII and/or activated Factor XII with one (or more) binding domains and can specifically bind to another protein with another binding domain.

In one example, the protein is monospecific, bispecific, or multispecific. For example, the protein comprises an antigen binding domain which can specifically bind to Factor XII and/or activated Factor XII and an antigen binding domain that specifically binds to another protein.

In one example, the antigen binding domain is monospecific, bispecific, or multispecific. For example, the antigen binding domain is monospecific. In one example, the antigen binding domain is multispecific, for example, the antigen binding domain is bispecific.

In one example, the antigen binding domain is monospecific.

In one example, the antigen binding domain is not bispecific.

In one example, the protein comprises an antibody variable region that competitively inhibits the binding of antibody 3F7 comprising a heavy chain variable region (V_{H}) comprising a sequence set forth in SEQ ID NO: 1 and a light chain variable region (V_{L}) comprising a sequence set forth in SEQ ID NO: 2 to Factor XII.

In one example, the protein comprises an antibody variable region that competitively inhibits the binding of germlined antibody 3F7 (3F7G) comprising a heavy chain variable region (V_{H}) comprising a sequence set forth in SEQ ID NO: 3 and a light chain variable region (V_{L}) comprising a sequence set forth in SEQ ID NO: 4 to Factor XII.

In one example, the protein comprises an antibody variable region that competitively inhibits the binding of affinity matured antibody 3F7 (3F7^{aff}) comprising a heavy chain variable region (V_{H}) comprising a sequence set forth in SEQ ID NO: 5 and a light chain variable region (V_{L}) comprising a sequence set forth in SEQ ID NO: 6 to Factor XII.

In one example, the protein comprises an antibody variable region that competitively inhibits the binding of garadacimab to Factor XII and/or to activated Factor XII.

In one example, the protein comprises a heavy chain variable region (V_{H}) comprising an amino acid sequence set forth in SEQ ID NO: 1 and a light chain variable region (V_{L}) comprising an amino acid sequence set forth in SEQ ID NO: 2.

In one example, the protein is an antibody or antigen binding fragment thereof comprising a heavy chain variable region (V_{H}) comprising the complementarity determining regions (CDRs) of a V_{H} comprising an amino acid sequence set forth in SEQ ID NO: 1 and a light chain variable region (V_{L}) comprising the CDRs of a V_{L} comprising an amino acid sequence set forth in SEQ ID NO: 2.

For example, the protein comprises:
(i) a V_{H} comprising:
   (a) a CDR1 comprising a sequence set forth in amino acids 25-34 of SEQ ID NO: 1;
   (b) a CDR2 comprising a sequence set forth in amino acids 49-65 of SEQ ID NO: 1; and
   (c) a CDR3 comprising a sequence set forth in amino acids 98-108 of SEQ ID NO: 1;
and/or
(ii) a V_{L} comprising:
   (a) a CDR1 comprising a sequence set forth in amino acids 23-33 of SEQ ID NO: 2;
   (b) a CDR2 comprising a sequence set forth in amino acids 49-55 of SEQ ID NO: 2; and
   (c) a CDR3 comprising a sequence set forth in amino acids 88-96 of SEQ ID NO: 2.

In one example, the protein comprises:
(i) a V_{H} comprising:
   (a) a sequence set forth in SEQ ID NO: 1; or
   (b) a CDR1 comprising a sequence set forth in SEQ ID NO: 7; a CDR2 comprising a sequence set forth in SEQ ID NO: 8; and a CDR3 comprising a sequence set forth in SEQ ID NO: 9; or
   (c) a CDR1 comprising a sequence set forth in SEQ ID NO: 7; a CDR2 comprising a sequence set forth in SEQ ID NO: 10; and a CDR3 comprising a sequence set forth in SEQ ID NO: 11;
and/or
(ii) a V_{L} comprising:
   (a) a sequence set forth in SEQ ID NO: 2; or
   (b) a CDR1 comprising a sequence set forth in SEQ ID NO: 12; a CDR2 comprising a sequence set forth in SEQ ID NO: 13; and a CDR3 comprising a sequence set forth in SEQ ID NO: 14; or
   (c) a CDR1 comprising a sequence set forth in SEQ ID NO: 12; a CDR2 comprising a sequence set forth in SEQ ID NO: 13; and a CDR3 comprising a sequence set forth in SEQ ID NO: 15.

In one example, the protein is an antibody comprising:
(i) a V_{H} comprising:
   (a) a CDR1 comprising a sequence set forth in SEQ ID NO: 7;
   (b) a CDR2 comprising a sequence set forth in SEQ ID NO: 8; and
   (c) a CDR3 comprising a sequence set forth in SEQ ID NO: 9;
and/or
(ii) a V_{L} comprising:
   (a) a CDR1 comprising a sequence as set forth in SEQ ID NO: 12;
   (b) a CDR2 comprising a sequence as set forth in SEQ ID NO: 13; and
   (c) a CDR3 comprising a sequence as set forth in SEQ ID NO: 14.

In one example, the protein is an antibody comprising:
(i) a V_{H} comprising:
   (a) a CDR1 comprising a sequence set forth in SEQ ID NO: 7;
   (b) a CDR2 comprising a sequence set forth in SEQ ID NO: 10; and
   (c) a CDR3 comprising a sequence set forth in SEQ ID NO: 11;
and/or
(ii) a V_{L} comprising:
   (a) a CDR1 comprising a sequence as set forth in SEQ ID NO: 12;
   (b) a CDR2 comprising a sequence as set forth in SEQ ID NO: 13; and
   (c) a CDR3 comprising a sequence as set forth in SEQ ID NO: 15.

In one example, the protein comprises a V_{H} comprising a CDR2 as set forth in SEQ ID NO: 10.

In one example, the amino acid sequence of V_{H} CDR2 comprises Arginine (R), Asparagine (N) or Aspartic Acid (D) at position 3 and/or Proline (P), Valine (V), Isoleucine (I) or Methionine (M) at position 4 and/or Serine (S), Proline (P) or Alanine (A) at position 5 and/or Glycine (G), Leucine (L), Valine (V) or Threonine (T) at position 6 and/or any amino acid at position 7 and/or Threonine (T), Glycine (G) or Serine (S) at position 8.

In one example, the amino acid sequence of V_{H} CDR2 comprises Asparagine (N) at position 3 and Valine (V) at position 4 and Proline (P) at position 5 and Leucine (L) at position 6 and Tyrosine (Y) at position 7 and Glycine (G) at position 8.

In one example, the amino acid sequence of V_{H} CDR2 comprises Asparagine (N) at position 3 and Valine (V) at position 4 and Proline (P) at position 5 and Valine (V) at position 6 and Glutamine (Q) at position 7 and Glycine (G) at position 8.

In one example, the amino acid sequence of V_{H} CDR2 comprises Aspartic acid (D) at position 3 and Isoleucine (I) at position 4 and Proline (P) at position 5 and Threonine (T) at position 6 and Lysine (K) at position 7 and Glycine (G) at position 8.

In one example, the amino acid sequence of V_{H} CDR2 comprises Aspartic acid (D) at position 3 and Methionine (M) at position 4 and Proline (P) at position 5 and Threonine (T) at position 6 and Lysine (K) at position 7 and Glycine (G) at position 8.

In one example, the protein is an antibody comprising:
(i) a V_{H} comprising
   (a) a CDR1 set forth in SEQ ID NO: 7;
   (b) a CDR2 set forth in SEQ ID NO: 10 wherein the X at position 3 is D, the X at position 4 is I, the X at position 5 is P, the X at position 6 is T, the X at position 7 is K, and the X at position 8 is G; and
   (c) a CDR3 set forth in SEQ ID NO: 9;
and/or
(ii) a V_{L} comprising
   (a) a CDR1 set forth in SEQ ID NO: 12;
   (b) CDR2 set forth in SEQ ID NO: 13; and
   (c) a CDR3 set forth in SEQ ID NO: 14.

In one example, the protein comprises a V_{H} comprising a CDR3 as set forth in SEQ ID NO: 11.

In one example, the amino acid sequence of V_{H} CDR3 comprises Isoleucine (I), Methionine (M) or Valine (V) at position 9 and/or Serine (S) or Lysine (K) at position 10 and/or Proline (P), Lysine (K), Threonine (T) or Histidine (H) at position 11 and/or Histidine (H), Asparagine (N), Glycine (G) or Glutamine (Q) at position 12.

In one example, the protein comprises a V_{L} comprising a CDR3 as set forth in SEQ ID NO: 15.

In one example, the amino acid sequence of V_{L} CDR3 comprises Alanine (A) or Serine (S) at position 2, and/or Aspartic acid (D), Tyrosine (Y), Glutamic acid (E), Threonine (T), Tryptophan (W) or Serine (S) at position 4 and/or Alanine (A), Asparagine (N), Isoleucine (I), Leucine (L), Valine (V), Proline (P), Glutamine (Q) or Glutamic acid (E) at position 5 and/or Serine (S), Aspartic acid (D), Proline (P), Glutamic acid (E), Glutamine (Q) or Arginine (R) at position 6 and/or Leucine (L) or Valine (V) at position 7 and/or Glycine (G), Leucine (L) or Lysine (K) at position 9 and/or Valine (V), Alanine (A), Aspartic acid (D), Threonine (T), Methionine (M) or Glycine (G) at position 10.

In one example, the protein comprises a heavy chain variable region (V_{H}) comprising an amino acid sequence set forth in SEQ ID NO: 3 and a light chain variable region (V_{L}) comprising an amino acid sequence set forth in SEQ ID NO: 4.

In one example, the protein is an antibody or antigen binding fragment thereof comprising a V_{H} comprising the complementarity determining regions (CDRs) of a V_{H} comprising an amino acid sequence set forth in SEQ ID NO: 3 and a V_{L} comprising the CDRs of a V_{L} comprising an amino acid sequence set forth in SEQ ID NO: 4. For example, the protein comprises:
(i) a V_{H} comprising:
   (a) a CDR1 comprising a sequence set forth in amino acids 25-34 of SEQ ID NO: 3;
   (b) a CDR2 comprising a sequence set forth in amino acids 49-65 of SEQ ID NO: 3; and
   (c) a CDR3 comprising a sequence set forth in amino acids 98-108 of SEQ ID NO: 3;
and/or
(ii) a V_{L} comprising:
   (a) a CDR1 comprising a sequence set forth in amino acids 23-33 of SEQ ID NO: 4;
   (b) a CDR2 comprising a sequence set forth in amino acids 49-55 of SEQ ID NO: 4; and
   (c) a CDR3 comprising a sequence set forth in amino acids 88-96 of SEQ ID NO: 4.

In one example, the protein of the present disclosure is an affinity matured, chimeric, CDR grafted, or humanised antibody, or antigen binding fragment thereof. In one example, the protein is an affinity matured form of antibody 3F7. In one example, the protein is garadacimab.

In one example, the protein comprises a heavy chain variable region (V_{H}) comprising an amino acid sequence set forth in SEQ ID NO: 5 and a light chain variable region (V_{L}) comprising an amino acid sequence set forth in SEQ ID NO: 6.

In one example, the protein is an antibody or antigen binding fragment thereof comprising a V_{H} comprising the complementarity determining regions (CDRs) of a V_{H} comprising an amino acid sequence set forth in SEQ ID NO: 5 and a V_{L} comprising the CDRs of a V_{L} comprising an amino acid sequence set forth in SEQ ID NO: 6.

In one example, the protein comprises:
(i) a V_{H} comprising:
   (a) a CDR1 comprising a sequence set forth in amino acids 25-34 of SEQ ID NO: 5;
   (b) a CDR2 comprising a sequence set forth in amino acids 49-65 of SEQ ID NO: 5; and
   (c) a CDR3 comprising a sequence set forth in amino acids 98-108 of SEQ ID NO: 5;
and/or
(ii) a V_{L} comprising:
   (a) a CDR1 comprising a sequence set forth in amino acids 23-33 of SEQ ID NO: 6;
   (b) a CDR2 comprising a sequence set forth in amino acids 49-55 of SEQ ID NO: 6; and
   (c) a CDR3 comprising a sequence set forth in amino acids 88-96 of SEQ ID NO: 6.

In one example, the protein comprises:
(i) a V_{H} comprising:
   (a) a CDR1 comprising a sequence set forth in SEQ ID NO: 7;
   (b) a CDR2 comprising a sequence set forth in SEQ ID NO: 16; and
   (c) a CDR3 comprising a sequence set forth in SEQ ID NO: 9; and/or
(ii) a V_{L} comprising:
   (a) a CDR1 comprising a sequence as set forth in SEQ ID NO: 12;
   (b) a CDR2 comprising a sequence as set forth in SEQ ID NO: 13; and
   (c) a CDR3 comprising a sequence as set forth in SEQ ID NO: 14.

In one example, the protein, antibody or antigen binding fragment thereof is any form of the protein, antibody or functional fragment thereof encoded by a nucleic acid encoding any of the foregoing proteins, antibodies or functional fragments.

In one example, the organic acid buffer is selected from the group consisting of a histidine buffer, a glutamate buffer, a succinate buffer and a citrate buffer. In one example, the organic acid buffer is selected from the group consisting of a histidine buffer and a glutamate buffer.

In one example, the organic acid buffer is an amino acid buffer. For example, the amino acid buffer is selected from the group consisting of a histidine buffer and a glutamate buffer.

Advantageously, histidine buffer and glutamate buffer have higher thermal and aggregation stability (i.e., reduced propensity towards aggregation) compared to citrate buffer and/or succinate buffer.

In one example, the organic acid buffer is a histidine buffer. Suitable histidine buffers for use in the present disclosure will be apparent to the skilled person and included, for example, histidine chloride, histidine acetate, histidine phosphate and histidine sulfate. In one example, the histidine buffer is L-histidine.

In one example, the organic acid buffer is a glutamate buffer. Suitable glutamate buffers for use in the present disclosure will be apparent to the skilled person and include, for example, monosodium glutamate.

In one example, the organic acid buffer is a succinate buffer. Suitable succinate buffers for use in the present disclosure will be apparent to the skilled person and include, for example, succinic acid-monosodium succinate mixture, succinic acid-sodium hydroxide mixture, succinic acid-disodium succinate mixture.

In one example, the organic acid buffer is a citrate buffer. Suitable citrate buffers for use in the present disclosure will be apparent to the skilled person and include, for example, monosodium citrate-disodium citrate mixture, citric acid-trisodium citrate mixture, citric acid monosodium citrate mixture.

It will be apparent to the skilled person that buffers suitable for use in the present disclosure will provide sufficient buffer capacity to maintain the desired pH over the range of conditions to which it will be exposed during formulation and storage of the product. In one example, the formulation of the present disclosure has a pH of about 5.0 to about 7.0. For example, the formulation has a pH of about 5.0 to 6.5, or a pH of about 5.8 to about 6.4. For example, the organic acid buffer is a histidine buffer having a pH of about 5.5 to about 5.7. In one example, the organic acid buffer is a glutamate buffer having a pH of about 5.5. In one example, the organic acid buffer is a succinate buffer having a pH of about 5.5. In one example, the organic acid buffer is a citrate buffer having a pH of about 5.5. In one example, the organic acid buffer has a pH of about 5.5 to 6.5, or a pH of about 5.6 to about 6.4, or a pH of about 5.8 to about 6.4. In one example, the formulation has a pH of 5.8 to 6.4. For example, the formulation has a pH of about 5.8, or about 5.9, or about 6.0, or about 6.1, or about 6.2, or about 6.3, or about 6.4. In one example, the organic acid buffer is a histidine buffer and the formulation has a pH of about 5.8 to about 6.4. In one example, the organic acid buffer is a glutamate buffer and the formulation has a pH of about 5.8 to about 6.4. In one example, the organic acid buffer is a succinate buffer and the formulation has a pH of about 5.8 to about 6.4. In one example, the organic acid buffer is a citrate buffer and the formulation has a pH of about 5.8 to about 6.4.

In one example, the concentration of the organic acid buffer in the pharmaceutical formulation of the present disclosure is between about 2 mM and 120 mM. In one example, the organic acid buffer is present at a concentration of a least 2 mM. For example, the organic acid buffer is present at a concentration of between about 2 mM and about 10 mM. For example, the organic acid buffer is present at a concentration of about 2 mM, or about 3 mM, or about 4 mM, or about 5 mM, or about 6 mM, or about 7 mM, or about 8 mM, or about 9 mM, or about 10 mM. In one example, the organic acid buffer is present at a concentration of at least about 10 mM. For example, the organic acid buffer is present at a concentration of between about 10 mM and about 30 mM. For example, the organic acid buffer is present at a concentration of about 10 mM, or about 12 mM, or about 14 mM, or about 16 mM, or about 18 mM, or about 20 mM, or about 25 mM, or about 30 mM. In one example, the organic acid buffer is present at a concentration of between about 12 mM and about 25 mM. For example, the organic acid buffer is present at a concentration of about 20 mM. For example, the organic acid buffer is present at a concentration of between about 10 mM and about 60 mM. For example, the organic acid buffer is present at a concentration of about 10 mM, or about 15 mM, or about 20 mM, or about 25 mM, or about 30 mM, or about 35 mM, or about 40 mM, or about 45 mM, or about 50 mM, or about 55 mM, or about 60 mM. In one example, the organic acid buffer is present at a concentration of about 20 mM. In one example, the organic acid buffer is present at a concentration of between about 60 mM and about 120 mM. For example, the organic acid buffer is present at a concentration of about 60 mM, or about 65 mM, or about 70 mM, or about 75 mM, or about 80 mM, or about 85 mM, or about 90 mM, or about 95 mM or about 100 mM, or about 105 mM, or about 110 mM, or about 115 mM, or about 120 mM. In one example, the organic acid buffer is present at a concentration of about 100 mM.

In one example, the organic acid buffer is L-histidine and is present at a concentration of about 12 mM to about 25 mM. In one example, the organic buffer is L-histidine and is present at a concentration of about 20 mM.

In one example, the non-ionic surfactant is selected from the group consisting of polyoxyethylensorbitan fatty acid esters (e.g., polysorbate 20 and polysorbate 80), polyethylene-polypropylene copolymers, polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS). For example, the non-ionic surfactant is selected form the group consisting of polyoxyethylensorbitan fatty acid esters and polyoxyethylene-polyoxypropylene copolymers.

In one example, the non-ionic surfactant is selected from the group consisting of polysorbate 20, polysorbate 80 and poloxamer 188. For example, the non-ionic surfactant is polysorbate 80.

In one example, the concentration of the non-ionic surfactant in the pharmaceutical formulation of the present disclosure is between about 0.01% (w/v) and about 1.00% (w/v). In one example, the non-ionic surfactant is present at a concentration of at least about 0.01% (w/v). For example, the non-ionic surfactant is present at a concentration of between about 0.01% (w/v) and about 0.10% (w/v). For example, the non-ionic surfactant is present at a concentration of about 0.01% (w/v), or about 0.02% (w/v), or about 0.03% (w/v), or about 0.04% (w/v), or about 0.05% (w/v), or about 0.06% (w/v), or about 0.07% (w/v), or about 0.08% (w/v), or about 0.09% (w/v), or about 0.10% (w/v). In one example, the non-ionic surfactant is present at a concentration of about 0.02% (w/v) or about 0.05% (w/v). For example, the non-ionic surfactant is present at a concentration of about 0.02% (w/v). In another example, the non-ionic surfactant is present at a concentration of about 0.05% (w/v). In one example, the non-ionic surfactant is present at a concentration of between about 0.01% (w/v) and about 0.03% (w/v). In one example, the non-ionic surfactant is polysorbate 80 and is present at a concentration of between about 0.01% (w/v) and about 0.03% (w/v). In one example, the non-ionic surfactant is polysorbate 80 and is present at a concentration of about 0.02% (w/v).

In one example, the pharmaceutical formulation comprises an amino acid stabilizer selected from the group consisting of glycine, alanine, valine, leucine, isoleucine, methionine, threonine, phenylalanine, tyrosine, serine, cysteine, histidine, tryptophan, proline, aspartic acid, glutamic acid, arginine, lysine, omithine and asparagine. For example, the amino acid stabiliser is selected from the group consisting of proline, arginine, salts thereof and a combination thereof. In one example, the amino acid stabiliser is a salt form of an amino acid discussed herein.

In one example, the amino acid stabilizer is proline. In one example, the amino acid stabiliser is L-proline.

In one example, the amino acid stabiliser is arginine. In one example, the amino acid stabiliser is L-arginine. In one example, the amino acid stabiliser is L-arginine monohydrochloride.

In one example, the formulation comprises proline and arginine. For example, the formulation comprises L-proline and L-arginine or L-arginine Monohydrochloride.

Advantageously, proline has a significant effect on thermal and aggregation stability (i.e., reduced propensity towards aggregation) compared to phenylalanine, arginine and sorbitol.

In one example, the concentration of the amino acid stabiliser in the pharmaceutical formulation of the present disclosure is between about 50 mM and about 250 mM. In one example, the amino acid stabiliser is present at a concentration of between about 90 mM and about 200 mM. For example, the amino acid stabiliser is present at a concentration of about 90 mM, or about 100 mM, or about 110 mM, or about 120 mM, or about 130 mM, or about 140 mM, or about 150 mM, or about 160 mM, or about 170 mM, or about 180 mM, or about 190 mM, or about 200 mM. In one example, the amino acid stabiliser is present at a concentration of between about 100 mM and about 160 mM. In another example, the amino acid stabiliser is present at a concentration of between about 90 mM and about 150 mM. For example, the amino acid stabiliser is present at a concentration of about 140 mM. In another example, the amino acid stabiliser is present at a concentration of about 150 mM.

Discussion of the foregoing concentrations also relates to a salt form of the amino acid stabiliser and the concentration recited herein is the concentration of the salt form of the amino acid rather the concentration of the amino acid per se.

In one example, the formulation comprises proline at a concentration of between 110 mM and 170 mM, for example at a concentration of about 140 mM or of about 150 mM. In another example, the formulation comprises proline at a concentration of between 90 mM and 150 mM, for example at a concentration of about 140 mM.

In one example, the formulation further comprises arginine at a concentration of between 110 mM and 170 mM, for example at a concentration of about 150 mM. In another example, the formulation further comprises arginine at a concentration of between 100 mM and 160 mM, for example at a concentration of about 150 mM. In one example, the arginine is a salt form of arginine, e.g., arginine monohydrochloride and the concentration recited herein is the concentration of the salt form of arginine rather the concentration of arginine per se.

In one example, the formulation comprises proline at a concentration of between 90 mM and 150 mM and arginine at a concentration of between 100 mM and 160 mM. For example, the formulation comprises 140 mM L-proline and 150 mM L-arginine monohydrochloride.

In one example, the pharmaceutical formulation further comprises a polyol. For example, the polyol is selected from a sugar, a sugar alcohol and a saccharic acid (i.e., an aldaric acid).

In one example, the polyol is a sugar. For example, the sugar is a reducing or a non-reducing sugar. In one example, the polyol is a non-reducing sugar selected from the group consisting of fructose, mannose, maltose, lactose, arabinose, xylose, ribose, rhamnose, galactose and glucose. In one example, the polyol is a reducing sugar selected from the group consisting of sucrose, trehalose, sorbose, melezitose, and raffinose.

In one example, the polyol is a sugar alcohol. For example, the sugar alcohol is selected from the group consisting of mannitol, xylitol, erythritol, threitol, sorbitol and glycerol. In one example, the sugar alcohol is sorbitol.

In one example, the polyol is a saccharic acid, such as L-gluconic acid and its metal salt.

In one example, the concentration of the polyol in the pharmaceutical formulation of the present disclosure is between about 50 mM and about 250 mM. In one example, the polyol is present at a concentration of between about 60 mM and about 140 mM. For example, the polyol is present at a concentration of about 60 mM, or about 70 mM, or about 80 mM, or about 90 mM, or about 100 mM, or about 110 mM, or about 120 mM, or about 130 mM, or about 140 mM. In one example, the polyol is present at a concentration of about 80 mM.

In one example, the formulation does not comprise a polyol. For example, the formulation does not comprise, a sugar, a sugar alcohol or a saccharic acid.

In one example, the formulation does not comprise a salt. For example, the formulation does not comprise, for example, sodium chloride, calcium chloride and/or potassium chloride. Discussion of the foregoing does not relate to a salt form of an amino acid as disclosed herein.

In one example, the formulation has a dynamic (i.e., absolute) viscosity of less than about 30 mPa*s at 20° C. For example, the formulation has a dynamic viscosity of about 30 mPa*s, or about 28 mPa*s, or about 26 mPa*s, or about 24 mPa*s, or about 22 mPa*s, or about 20 mPa*s at 20° C. In one example, the formulation has a dynamic (i.e., absolute) viscosity of less than about 20 mPa*s at 20° C. For example, the formulation has a dynamic viscosity of about 20 mPa*s, or about 19 mPa*s, or about 18 mPa*s, or about 17 mPa*s, or about 16 mPa*s, or about 15 mPa*s at 20° C. In one example, the formulation has a dynamic viscosity of less than about 15 mPa*s at 20° C. For example, the formulation has a dynamic viscosity of about 15 mPa*s, or about 14 mPa*s, or about 13 mPa*s, or about 12 mPa*s, or about 11 mPa*s, or about 10 mPa*s at 20° C. In one example, the formulation has a dynamic viscosity of less than about 10 mPa*s at 20° C. For example, the formulation has a dynamic viscosity of about 10 mPa*s, or about 9 mPa*s, or about 8 mPa*s, or about 7 mPa*s, or about 6 mPa*s, or about 5 mPa*s, or about 4 mPa*s, or about 3 mPa*s, or about 2 mPa*s at 20° C. In one example, the formulation has a dynamic viscosity of between about 3.0 mPa*s and about 4.0 mPa*s at 20° C. For example, the formulation has a dynamic viscosity of about 3.3 mPa*s at 20° C. In another example, the formulation has a dynamic viscosity of between about 8.0 mPa*s and about 10.0 mPa*s at 20° C. For example, the formulation has a dynamic viscosity of about 8.9 mPa*s at 20° C. In one example, the formulation has a dynamic viscosity of about 9 mPa*s at 20° C.

In one example, the formulation comprises the protein or antibody in an amount of about 100 mg/ml and has a dynamic viscosity of less than about 10.0 mPa*s at 20° C. For example, such formulation has a dynamic viscosity of about 3.3 mPa*s at 20° C.

In one example, the formulation comprises the protein or antibody in an amount of about 170 mg/ml and has a dynamic viscosity of less than about 10.0 mPa*s at 20° C. For example, such formulation has a dynamic viscosity of about 8.9 mPa*s at 20° C.

In one example, the formulation has a dynamic (i.e., absolute) viscosity of less than about 30 mPa*s at 25° C. For example, the formulation has a dynamic viscosity of about 30 mPa*s, or about 28 mPa*s, or about 26 mPa*s, or about 24 mPa*s, or about 22 mPa*s, or about 20 mPa*s at 25° C. In one example, the formulation has a dynamic (i.e., absolute) viscosity of less than about 20 mPa*s at 25° C. For example, the formulation has a dynamic viscosity of about 20 mPa*s, or about 19 mPa*s, or about 18 mPa*s, or about 17 mPa*s, or about 16 mPa*s, or about 15 mPa*s at 25° C. In one example, the formulation has a dynamic viscosity of less than about 15 mPa*s at 25° C. For example, the formulation has a dynamic viscosity of about 15 mPa*s, or about 14 mPa*s, or about 13 mPa*s, or about 12 mPa*s, or about 11 mPa*s, or about 10 mPa*s at 25° C. In one example, the formulation has a dynamic viscosity of less than about 10 mPa*s at 25° C. For example, the formulation has a dynamic viscosity of about 10 mPa*s, or about 9 mPa*s, or about 8 mPa*s, or about 7 mPa*s, or about 6 mPa*s, or about 5 mPa*s, or about 4 mPa*s, or about 3 mPa*s, or about 2 mPa*s at 25° C. In one example, the formulation has a dynamic viscosity of between about 2 mPa*s and about 9 mPa*s at 25° C. In one example, the formulation has a dynamic viscosity of between about 1.0 mPa*s and about 3.0 mPa*s at 25° C. For example, the formulation has a dynamic viscosity of about 2.8 mPa*s at 25° C. In another example, the formulation has a dynamic viscosity of between about 7.0 mPa*s and about 8.0 mPa*s at 25° C. For example, the formulation has a dynamic viscosity of about 7.5 mPa*s at 25° C.

In one example, the formulation comprises the protein or antibody in an amount of about 100 mg/ml and has a dynamic viscosity of less than about 10.0 mPa*s at 25° C. For example, such formulation has a dynamic viscosity of about 2.8 mPa*s at 25° C.

In one example, the formulation comprises the protein or antibody in an amount of about 170 mg/ml and has a dynamic viscosity of less than about 10.0 mPa*s at 25° C. For example, such formulation has a dynamic viscosity of about 7.5 mPa*s at 25° C.

Methods of assessing viscosity will be apparent to the skilled person and/or are described herein. For example, viscosity may be assessed by use of a microviscometer, such as a rolling-ball viscometer. A rolling-ball viscometer measures the rolling time of a ball through transparent and opaque liquids according to Hoppler's falling ball principle. An example of a rolling-ball viscometer is the Anton Par Lovis 2000 M Microviscometer.

In one example, the formulation has a density of between about 1.00 to about 1.10 g/cm³ at 20° C. For example, the density of the formulation is about 1.01 g/cm³ or about 1.02 g/cm³, or about 1.03 g/cm³, or about 1.04 g/cm³, or about 1.05 g/cm³, or about 1.06 g/cm³, or about 1.07 g/cm³, or about 1.08 g/cm³, or about 1.09 g/cm³, or about 1.10 g/cm³ at 20° C. In one example, the density of the formulation is about 1.04 g/cm³, or about 1.05 g/cm³, or about 1.06 g/cm³, or about 1.07 g/cm³. For example, the density of the formulation is about 1.06 g/cm³. Methods of assessing density of the formulation will be apparent to the skilled person and/or described herein. In one example, density is determined using a density meter, for example the Mettler Toledo DA-100M Density Meter.

In one example, the present disclosure provides a pharmaceutical formulation comprising a protein comprising an antigen binding domain that binds to or specifically binds to Factor XII and/or an activated form thereof, an organic acid buffer selected from the group consisting of a histidine buffer and a glutamate buffer, an amino acid stabiliser selected from the group consisting of proline, arginine, salts thereof and a combination thereof, polysorbate 80 as a non-ionic surfactant, wherein the formulation has a viscosity of less than about 10 mPa*s at 20° C.

In one example, the formulation comprises a protein comprising an antigen binding domain that binds to or specifically binds to Factor XII and/or an activated form thereof, a histidine buffer or a glutamate buffer, proline and polysorbate 80. For example, the formulation comprises the antibody or antigen binding fragment thereof, a histidine buffer, proline and polysorbate 80. In another example, the formulation comprises the antibody or antigen binding fragment thereof, a glutamate buffer, proline and polysorbate 80. Optionally, the formulation further comprises arginine or sorbitol.

The present disclosure provides a liquid pharmaceutical formulation comprising about 170 mg/ml of an antibody or antigen binding fragment thereof, a histidine buffer, polysorbate 80 and proline as a stabiliser, wherein the formulation has a pH of 5.5 to 6.5 and a viscosity of less than about 10 mPa*s at 20° C. For example, the formulation comprises about 20 mM L-histidine buffer having a pH of 5.8 to 6.4, 0.02% (w/v) polysorbate 80 and 140 mM or 150 mM L-proline. In one example, the formulation further comprises 150 mM L-arginine monohydrochloride. In another example, the formulation further comprises 80 mM sorbitol.

The present disclosure provides a liquid pharmaceutical formulation comprising between about 100 mg/ml and about 110 mg/ml of a protein comprising an antigen binding domain of the present disclosure, a L-histidine buffer, polysorbate 80 and L-proline and L-arginine monohydrochloride as stabilisers, wherein the formulation has a pH of 5.5 to 6.5 and a viscosity of less than about 10 mPa*s at 20° C.

The present disclosure provides a liquid pharmaceutical formulation comprising between about 160 mg/ml and about 180 mg/ml of a protein comprising an antigen binding domain of the present disclosure, a L-histidine buffer, polysorbate 80 and L-proline and L-arginine monohydrochloride as stabilisers, wherein the formulation has a pH of 5.5 to 6.5 and a viscosity of less than about 10 mPa*s at 20° C.

The present disclosure also provides a pharmaceutical formulation comprising about 170 mg/ml of an antibody or antigen binding fragment thereof described herein, a glutamate buffer, polysorbate 80 and proline as a stabiliser, wherein the formulation has a pH of 5.5 to 6.5 and a viscosity of less than about 10 mPa*s at 20° C. For example, the formulation comprises about 100 mM glutamate buffer having a pH of 5.5, 0.05% (w/v) polysorbate 80 and 150 mM proline.

The present disclosure provides a pharmaceutical formulation having a pH of 5.5 to 6.5 and comprising about 160 mg/ml to about 180 mg/ml of an antibody or antigen binding fragment thereof described herein, a histidine buffer, polysorbate 80 and proline and arginine monohydrochloride as stabilisers, wherein the formulation has a viscosity of less than about 10 mPa*s at 20° C. and 25° C. For example, the formulation has a pH of 5.8 to 6.4 and comprises about 12 to about 25 mM L-histidine buffer, 0.01% to 0.03% (w/v) polysorbate 80, 110 mM to 170 mM L-proline and 110 mM to 170 mM L-arginine. In one example, the formulation comprises 12 to 25 mM L-histidine buffer having a pH of 5.8 to 6.4, 0.01% to 0.03% (w/v) polysorbate 80, 90 mM to 150 mM L-proline and 100 mM to 160 mM L-arginine. In one example, the osmolality of the formulation is about 430-530 mOsm/kg, for example about 450 mOsm/kg. In one example, the antibody comprises a V_{H} comprising an amino acid sequence set forth in SEQ ID NO: 5 and a V_{L} comprising an amino acid sequence set forth in SEQ ID NO: 6.

The present disclosure provides a pharmaceutical formulation having a pH of 5.5 to 6.5 and comprising about 170 mg/ml of an antibody or antigen binding fragment thereof described herein, a histidine buffer, polysorbate 80 and proline and arginine as stabilisers, wherein the formulation has a viscosity of less than about 10 mPa*s at 20° C. and 25° C. For example, the formulation has a pH of 5.8 to 6.4 and comprises about 20 mM L-histidine buffer, 0.02% (w/v) polysorbate 80, 140 mM L-proline and 150 mM L-arginine. In one example, the osmolality of the formulation is about 450 mOsm/kg. In one example, the antibody comprises a V_{H} comprising an amino acid sequence set forth in SEQ ID NO: 5 and a V_{L} comprising an amino acid sequence set forth in SEQ ID NO: 6.

The present disclosure provides a pharmaceutical formulation having a pH of 5.5 to 6.5 and comprising about 100 mg/ml of an antibody or antigen binding fragment thereof described herein, a histidine buffer, polysorbate 80 and proline and arginine monohydrochloride as stabilisers, wherein the formulation has a viscosity of less than about 10 mPa*s at 20° C. and 25° C. For example, the formulation has a pH of 5.8 to 6.4 and comprises between 12-25 mM histidine buffer, 0.01% to 0.03% (w/v) polysorbate 80, 110 mM to 170 mM proline and 110 mM to 170 mM arginine. In one example, the osmolality of the formulation is about 430 mOsm/kg. In one example, the antibody comprises a V_{H} comprising an amino acid sequence set forth in SEQ ID NO: 5 and a V_{L} comprising an amino acid sequence set forth in SEQ ID NO: 6.

The present disclosure provides a pharmaceutical formulation comprising about 100 mg/ml of an antibody or antigen binding fragment thereof described herein, a histidine buffer having a pH of 5.5 to 6.5, polysorbate 80 and proline and arginine as stabilisers, wherein the formulation has a viscosity of less than about 5 mPa*s at 20° C. and 25° C. For example, the formulation comprises about 20 mM histidine buffer having a pH of 5.8 to 6.4, 0.02% (w/v) polysorbate 80, 140 mM proline and 150 mM arginine. In one example, the osmolality of the formulation is about 430 mOsm/kg. In one example, the antibody comprises a V_{H} comprising an amino acid sequence set forth in SEQ ID NO: 5 and a V_{L} comprising an amino acid sequence set forth in SEQ ID NO: 6.

The present disclosure provides a pharmaceutical formulation having a pH of 5.5 to 6.5 and comprising between about 100 mg/ml and about 170 mg/ml of a protein comprising an antigen binding domain described herein, a histidine buffer, polysorbate 80 and proline and arginine monohydrochloride as stabilisers, wherein the formulation has a viscosity of less than about 30 mPa*s at 20° C. and wherein the protein comprises a V_{H} comprising an amino acid sequence set forth in SEQ ID NO: 5 and a V_{L} comprising an amino acid sequence set forth in SEQ ID NO: 6. For example, the formulation has a pH of 5.8 to 6.4 and comprises about 20 mM L-histidine buffer, 0.02% (w/v) polysorbate 80, 140 mM L-proline and 150 mM L-arginine monohydrochloride. In one example, the osmolality of the formulation is about 430 to about 450 mOsm/kg.

The present disclosure also provides a pharmaceutical formulation having a pH of 5.5 to 6.5 and comprising between about 100 mg/ml and about 170 mg/ml of a protein comprising an antigen binding domain as described herein, a histidine buffer, polysorbate 80 and proline and arginine monohydrochloride as stabilisers, wherein the formulation has a viscosity of less than about 30 mPa*s at 20° C. and wherein the protein comprises:
(i) a V_{H} comprising a CDR1 comprising a sequence set forth in SEQ ID NO: 7; a CDR2 comprising a sequence set forth in SEQ ID NO: 16; and a CDR3 comprising a sequence set forth in SEQ ID NO: 9; and
(ii) a V_{L} comprising a CDR1 comprising a sequence set forth in SEQ ID NO: 12; a CDR2 comprising a sequence set forth in SEQ ID NO: 13; and a CDR3 comprising a sequence set forth in SEQ ID NO: 14.

In one example, the high concentration formulation of the present disclosure is a stable formulation. For example, the formulation is physically and/or thermally stable.

In one example, the formulation of the present disclosure is a liquid formulation. For example, the formulation is an aqueous formulation.

In one example, the formulation has not previously been lyophilised. In one example, the formulation is not a reconstituted formulation. For example, the formulation is a liquid formulation.

Methods of assessing thermal stability will be apparent to the skilled person and/or described herein. In one example, thermal aggregation stability is determined by differential scanning fluorimetry (DSF). For example, changes in intrinsic protein fluorescence are monitored across a range of temperatures, (e.g., 20-95° C.; with a temperature increase at the rate of e.g., 0.5° C./min) to determine the midpoint of thermal transition (Tₘ; i.e., melting temperature) and onset of melting temperature (Tₒₙₛₑₜ). Static light scattering is also monitored at 266 nm and 473 nm to determine the onset of aggregation temperature (T_{agg}).

In one example, the Tₘ of the formulation, as determined by differential scanning fluorimetry, is between about 55.0° C. and about 70.0° C., such as between about 59° C. and about 67° C. For example, the Tₘ of the formulation is about 59.0° C., or about 60.0° C., or about 61.0° C., or about 62.0° C., or about 63.0° C., or about 64.0° C., or about 65.0° C., or about 66.0° C., or about 67° C.

In one example, the Tₒₙₛₑₜ of the formulation, as determined by differential scanning fluorimetry, is between about 55.0° C. and about 70.0° C., such as between about 58.0° C. and 63.0° C. For example, the Tₒₙₛₑₜ of the formulation is about 58.0° C., or about 59.0° C., or about 60.0° C., or about 61.0° C., or about 62.0° C., or about 63.0° C.

In one example, the T_{agg} of the formulation at 266 nm, as determined by differential scanning fluorimetry, is between about 56.0° C. and about 65.0° C. For example, the T_{agg} at 266 nm of the formulation is about 56.0° C., or about 57.0° C., or about 58.0° C., or about 60.0° C., or about 61.0° C., or about 62.0° C., or about 63.0° C., or about 64.0° C., or about 65.0° C.

In one example, the T_{agg} of the formulation at 473 nm, as determined by differential scanning fluorimetry, is between about 58.0° C. and about 64.0° C. For example, the T_{agg} at 266 nm of the formulation is about 58.0° C., or about 60.0° C., or about 61.0° C., or about 62.0° C., or about 63.0° C., or about 64.0° C.

In one example, the formation of aggregates (i.e., particle size distribution) of the antibody or antigen binding fragment thereof is assessed using dynamic light scattering (DLS). For example, the fluctuation of light intensity using a digital correlator (e.g., Malvern Zetasizer software) is measured and the Z-average hydrodynamic diameter and polydispersity index (using e.g., a cumulants analysis) are determined. In one example, the organic acid buffer does not significantly change (i.e., increase or decrease) the Z-average hydrodynamic diameter of the antibody or antigen binding fragment.

The stability of the formulation may also be assessed by measuring total aggregates and/or monomer content. Methods for assessing accumulation of aggregates and monomer content of the formulation will be apparent to the skilled person and/or described herein. In one example, the percent total aggregates of antibody or antigen binding fragment thereof in the formulation is determined by size-exclusion chromatography (SEC or SE-HPLC). In another example, the percent monomer of antibody or antigen binding fragment thereof in the formulation is determined by size-exclusion chromatography (SEC or SE-HPLC). For example, a formulation of the present disclosure comprises at least 90% monomer antibody or antigen binding fragment thereof and/or less than (i.e., no more than) 10% aggregate and/or degraded (e.g., fragmented) antibody or antigen binding fragment thereof. In one example, a formulation of the present disclosure comprises at least 95% monomer antibody or antigen binding fragment thereof and/or less than (i.e., no more than) 5% aggregate and/or degraded (e.g., fragmented) antibody or antigen binding fragment thereof.

In one example, the formulation comprises less than about 10% total aggregates of the antibody. For example, the formulation comprises less than about 10%, or less than about 9%, or less than about 8%, or less than about 7%, or less than about 6%, or less than about 5%, or less than about 4%, or less than about 3%, or less than about 2%, or less than about 1% total aggregates. For example, the aggregates in the composition are high molecular weight species and/or degraded antibody or antigen binding fragment thereof.

In one example, the formulation comprises about 0.5% to about 3.0% total aggregates after 4-5 weeks storage at 5° C. For example, the formulation comprises about 0.5%, or about 1.0%, or about 1.5%, or about 2.0%, or about 2.5%, or about 3.0% total aggregates after 4-5 weeks storage at 5° C. In one example, the formulation comprises about 1.5% to about 2.0% total aggregates after 4-5 weeks storage at 5° C. For example, the formulation comprises about 1.7% total aggregates after 4-5 weeks storage at 5° C.

In one example, the formulation comprises less than about 10% total aggregates of the antibody after 24 months storage at 5° C. For example, the formulation comprises less than about 10%, or less than about 9%, or less than about 8%, or less than about 7%, or less than about 6%, or less than about 5%, or less than about 4%, or less than about 3%, or less than about 2%, or less than about 1% total aggregates after 24 months storage at 5° C. In one example, the formulation comprises about 1.5% to about 2.0% total aggregates after 24 months storage at 5° C. For example, the formulation comprises about 2.0% total aggregates after 24 months storage at 5° C. For example, the aggregates in the composition are high molecular weight species and/or degraded antibody or antigen binding fragment thereof.

In one example, the formulation comprises about 2.0% to about 7.0% total aggregates after 4-5 weeks storage at 35-40° C. For example, the formulation comprises about 2.0%, or about 2.5%, or about 3.0%, or about 3.5%, or about 4.0%, or about 4.5%, or about 5.0%, or about 5.5%, or about 6.0%, or about 6.5%, or about 7.0% total aggregates of the antibody after 4-5 weeks storage at 35-40° C.

In one example, the formulation comprises less than about 10% total aggregates of the antibody after 24 months storage at 25° C. For example, the formulation comprises less than about 10%, or less than about 9%, or less than about 8%, or less than about 7%, or less than about 6%, or less than about 5%, or less than about 4%, or less than about 3%, or less than about 2%, or less than about 1% total aggregates after 24 months storage at 5° C. In one example, the formulation comprises about 1.5% to about 3.5% total aggregates after 24 months storage at 25° C. For example, the formulation comprises about 3.1% total aggregates after 24 months storage at 25° C. For example, the aggregates in the composition are high molecular weight species and/or degraded antibody or antigen binding fragment thereof.

In one example, the formulation comprises less than about 10% total aggregates of the antibody after 36 months storage at 25° C. For example, the formulation comprises less than about 10%, or less than about 9%, or less than about 8%, or less than about 7%, or less than about 6%, or less than about 5%, or less than about 4%, or less than about 3% after 36 months storage at 5° C. In one example, the formulation comprises about 1% to about 5% total aggregates after 24 months storage at 25° C. For example, the formulation comprises about 2.4% total aggregates after 24 months storage at 25° C. For example, the aggregates in the composition are high molecular weight species and/or degraded antibody or antigen binding fragment thereof.

In one example, at least about 90% of the antibody in the formulation is a monomer. For example, at least about 90%, or about 91%, or about 92%, or about 93%, or about 94%, or about 95%, or about 96%, or about 97%, or about 98%, or about 99%, or about 99.5% of the antibody in the formulation is a monomer. In one example, at least about 95% of the antibody in the formulation is a monomer.

In one example, at least about 95% of the antibody in the formulation is a monomer after 4-5 weeks storage at 5° C. For example, at least about 95%, or about 95.5%, or about 96%, or about 96.5%, or about 97%, or about 97.5%, or about 98%, or about 98.5%, or about 99%, or about 99.5% of the antibody in the formulation is a monomer after 4-5 weeks storage at 5° C. In one example, about 98% to about 99% of the antibody in the formulation is a monomer after 4-5 weeks storage at 5° C. For example, about 98.3% of the antibody in the formulation is a monomer after 4-5 weeks storage at 5° C. In one example, about 98% of the antibody in the formulation is a monomer after 24 months storage at 5° C.

In one example, at least about 92% of the antibody in the formulation is a monomer after 4-5 weeks storage at 35-40° C. For example, at least about 92%, or about 92.5%, or about 93%, or about 93.5%, or about 94%, or about 94.5%, or about 95%, or about 95.5%, or about 96%, or about 96.5%, or about 97%, or about 97.5%, or about 98%, or about 98.5%, or about 99%, or about 99.5% of the antibody in the formulation is a monomer after 4-5 weeks storage at 35-40° C.

In one example, at least about 96% of the antibody in the formulation is a monomer after 24 months storage at 25° C. For example, at least about 96%, or about 96.5%, or about 97%, or about 97.5%, or about 98%, or about 98.5%, or about 99%, or about 99.5% of the antibody in the formulation is a monomer after 24 months storage at 25° C. For example, about 96.9% of the antibody in the formulation is monomer after 24 months storage at 25° C.

In one example, the osmolality of the formulation is between about 150 mOsm/kg and about 550 mOsm/kg. For example, the osmolality of the formulation is about 150 mOsm/kg, or about 175 mOsm/kg, or about 200 mOsm/kg, or about 225 mOsm/kg, or about 250 mOsm/kg, or about 275 mOsm/kg, or about 300 mOsm/kg, or about 325 mOsm/kg, or about 350 mOsm/kg, or about 375 mOsm/kg, or about 400 mOsm/kg, or about 425 mOsm/kg, or about 450 mOsm/kg, or about 475 mOsm/kg, or about 500 mOsm/kg, or about 550 mOsm/kg. In one example, the osmolality of the formulation is between about 400 mOsm/kg and about 550 mOsm/kg. For example, the osmolality of the formulation is about 400 mOsm/kg, or about 410 mOsm/kg, or about 420 mOsm/kg, or about 430 mOsm/kg, or about 440 mOsm/kg, or about 450 mOsm/kg, or about 460 mOsm/kg, or about 470 mOsm/kg, or about 480 mOsm/kg, or about 490 mOsm/kg, or about 500 mOsm/kg, or about 550 mOsm/kg. In one example, the osmolality is between about 400 mOsm/kg and about 500 mOsm/kg. For example, the osmolality is about 430 mOsm/kg. In one example, the osmolality of the formulation is about 450 mOsm/kg.

In one example, the present disclosure provides the pharmaceutical formulation of the present disclosure for use in treating or preventing a disease or condition in a subject.

In one example, the present disclosure provides the pharmaceutical formulation of the present disclosure for use in antagonising activity of Factor XII and/or activated Factor XII in a subject.

In one example, the present disclosure provides the pharmaceutical formulation of the present disclosure for use in antagonising activation of Factor XII and/or activated Factor XII in a subject.

The present disclosure also provides a method of treating or preventing a disease or condition in a subject, the method comprising administering the high concentration formulation of the present disclosure.

The present disclosure also provides a method of antagonising activity of Factor XII and/or activated Factor XII in a subject, the method comprising administering the high concentration formulation of the present disclosure.

The present disclosure also provides a method of antagonising activation of Factor XII and/or activated Factor XII in a subject, the method comprising administering the high concentration formulation of the present disclosure.

In one example, the present disclosure provides use of the pharmaceutical formulation of the present disclosure in the manufacture of a medicament for the treatment or prevention of a disease or condition in a subject.

In one example, the present disclosure provides use of the pharmaceutical formulation of the present disclosure in the manufacture of a medicament for antagonising activity of Factor XII and/or activated Factor XII in a subject.

In one example, the present disclosure provides use of the pharmaceutical formulation of the present disclosure in the manufacture of a medicament for antagonising activation of Factor XII and/or activated Factor XII in a subject.

In one example, the subject is in need of treatment with the pharmaceutical formulation of the present disclosure (i.e., in need thereof).

In one example, the disease or condition is a thrombotic disorder, an inflammatory disorder and/or a thrombo-inflammatory disorder. For example, the subject is suffering from, or at risk of a thrombotic disorder, an inflammatory disorder and/or a thrombo-inflammatory disorder.

In one example, the subject suffers from a thrombotic disorder, an inflammatory disorder and/or a thrombo-inflammatory disorder. In one example, the subject has been diagnosed as suffering from a thrombotic disorder, an inflammatory disorder and/or a thrombo-inflammatory disorder. In one example, the subject is receiving treatment of a thrombotic disorder, an inflammatory disorder and/or a thrombo-inflammatory disorder.

In one example of any method described herein, the pharmaceutical formulation of the present disclosure is administered before or after the development of a disease or condition, e.g., a thrombotic disorder, an inflammatory disorder and/or a thrombo-inflammatory disorder. In one example of any method described herein, the pharmaceutical formulation of the present disclosure is administered before the development of the disease or condition. In one example of any method described herein, the pharmaceutical formulation of the present disclosure is administered after the development of the disease or condition.

In one example, the pharmaceutical formulation is administered before or after the onset of symptoms of a disease or condition, e.g., a thrombotic disorder, an inflammatory disorder and/or a thrombo-inflammatory disorder. In one example, the pharmaceutical formulation is administered before the onset of symptoms of a disease or condition, e.g., a thrombotic disorder, an inflammatory disorder and/or a thrombo-inflammatory disorder. In one example, the pharmaceutical formulation is administered after the onset of symptoms of a disease or condition, e.g., a thrombotic disorder, an inflammatory disorder and/or a thrombo-inflammatory disorder. In one example, the pharmaceutical formulation is administered at a dose that alleviates or reduces one or more of the symptoms of a disease or condition, e.g., a thrombotic disorder, an inflammatory disorder and/or a thrombo-inflammatory disorder.

Symptoms of a thrombotic disorder, an inflammatory disorder and/or a thrombo-inflammatory disorder will be apparent to the skilled person and will be dependent on the condition. Exemplary symptoms of a condition or disorder include, for example:
Recurring infection;
Joint inflammation;
Muscle weakness;
Rash or discolouration of the skin;
Edema, especially in the extremities (e.g., feet, hands, legs or arms) or eyes;
Abdominal pain;
Pain, swelling and tenderness in the affected area;
A dull or heavy ache in the affected area;
Warm skin in the area of the thrombus;
Red skin;
Chest pain;
Breathing difficulties (e.g., breathlessness and/or chest tightness);
Sudden loss of strength in one arm or leg;
Nausea;
Fatigue;
Hematuria;
Partial or complete paralysis; and
Poor cognitive ability.

In one example, the thrombotic disorder, inflammatory disorder and/or thrombo-inflammatory disorder is selected from the group consisting of venous, arterial or capillary thrombus formation (such as stroke, myocardial infarction, deep vein thrombosis (DVT), portal vein thrombosis, renal vein thrombosis, jugular vein thrombosis, cerebral sinus thrombosis, Budd-Chiari syndrome, Paget-Schroetter disease or silent brain ischemia), thrombus formation in the heart, thromboembolism, thrombus formation during and/or after contacting blood of a human or animal subject with artificial surfaces, disseminated intravascular coagulation (DIC), atrial fibrillation, acute coronary syndromes (ACS), atherosclerotic disease, ischaemic stroke with reperfusion, a disease associated with ischemia-reperfusion injury (IRI, such as trauma, organ transplantation), neurotraumatic disorder (such as such as traumatic brain injury, spinal cord injury), a neurological inflammatory disease (such as multiples sclerosis), an interstitial lung disease (such as idiopathic pulmonary fibrosis (IPF)), pneumonia, fibrinolysis, a disease related to FXII/FXIIa-induced kinin formation (such as hereditary angioedema (HAE)), sepsis, a disease related to FXII/FXIIa-mediated complement activation, acute respiratory distress syndrome (ARDS), organ and cell transplantation, sickle cell disease and a condition associated with increased vascular permeability. fibrinolysis, and a condition associated with increased vascular permeability.

In one example, the disease or condition is a venous, arterial or capillary thrombus. For example, the venous or arterial thrombus is associated with a disease or condition selected from the group consisting of stroke, myocardial infarction, deep vein thrombosis (DVT), portal vein thrombosis, thromboembolism, renal vein thrombosis, jugular vein thrombosis, cerebral venous sinus thrombosis, Budd-Chiari syndrome, silent brain ischemia (SBI), and Paget-Schroetter disease.

In one example, the disease or condition is thrombus formation during and/or after contacting blood of a subject with artificial surfaces. In one example, the thrombus formation occurs during and/or after contacting blood of a human or animal subject with artificial surfaces during and/or after a medical procedure performed on a subject and a formulation of the disclosure is administered before and/or during and/or after said medical procedure. For example, in subjects with valve replacements, stents, percutaneous coronary intervention (PCI), extracorporeal membrane oxygenation (ECMO), or undergoing cardiopulmonary bypass surgery (CPB surgery).

In one example, the disease or condition is a chronic and/or an acute thromboembolism. For example, the chronic and/or acute thromboembolism is a pulmonary embolism, cerebral thromboembolism following atrial fibrillation-induced thrombus formation (e.g., stroke prevention in atrial fibrillation (SPAF)).

In one example, the stroke is thrombotic stroke. In another example, the stroke is stroke prevention in atrial fibrillation (SPAF).

In one example, the disease or condition is an ischemic stroke with reperfusion. For example, the disease or condition is a secondary aspect of stroke (e.g., the secondary aspects of ischemic or hemorrhagic stroke).

In one example, the disease or condition is a neurotraumatic disorder. For example, the neurotraumatic disorder is a traumatic injury of the central nervous system (CNS), including a spinal cord injury and a traumatic brain injury. In one example, the disease or condition is a spinal cord injury. In another example, the disease or condition is a traumatic brain injury.

In one example, the disease or condition is an ischemia-reperfusion injury (IRI). For example, the IRI is caused by a natural event (e.g., restoration of blood flow following a myocardial infarction), a trauma, or by one or more surgical procedures (e.g. organ transplantation) or other therapeutic interventions that restore blood flow to a tissue or organ that had been subjected to a diminished supply of blood. Such surgical procedures can include, for example, coronary artery bypass graft surgery, coronary angioplasty, organ transplant surgery, elective surgery, reconstructive surgery, vascular surgery, cardiac surgery, trauma surgery, crash or crush surgery, cancer surgery, orthopedic surgery, transplantation, or minimally invasive surgery. In one example, the surgical procedure can include the insertion of a device for delivery of a pharmacologically active substance, such as a thrombolytic agent or vasodilator, or a device to mechanically remove complete or partial obstructions, e.g., obstructions of blood vessels.

In one example, the thromboembolism is a pulmonary embolism. In another example, the thromboembolism is a systemic embolism. In a further example, the thromboembolism is chronic thromboembolic pulmonary hypertension.

In one example, the disease or condition is contact-mediated thrombo-inflammation.

In one example, the disease or condition is atrial fibrillation.

In one example, the disease or condition is an acute coronary syndrome (ACS).

In one example, the disease or condition is interstitial lung disease (ILD). For example, the interstitial lung disease is fibroproliferative and/or idiopathic pulmonary fibrosis. In one example, the disease or condition is idiopathic pulmonary fibrosis (IPF). For example, the subject suffers from idiopathic pulmonary fibrosis (IPF).

In one example, the disease or condition is an inflammatory disorder. For example, the inflammatory disorder is a neurological inflammatory disease (or neuroinflammatory disease). In one example, the neurological inflammatory disease is spinal cord injury (SCI), stroke, traumatic brain injury (TBI), secondary brain edema, edema of the central nervous system, multiple sclerosis (MS), transverse myelitis, or neuromyelitis optica (Devic's disease). In one example, the inflammatory disorder is pneumonia.

In one example, the disease or condition is fibrinolysis.

In one example, the disease or condition disorder is angiogenesis.

In one example, the disease or condition disorder is a disease related to FXII/FXIIa-induced kinin formation. For example, the disease or condition is selected from the group consisting of hereditary angioedema (HAE), bacterial infections of the lung, trypanosoma infections, hypotensive shock, pancreatitis, chagas disease, articular gout, arthritis, disseminated intravascular coagulation (DIC) and sepsis.

In one example, the disease or condition is hereditary angioedema (HAE). For example, the subject suffers from hereditary angioedema (HAE).

In one example, the formulation of the disclosure is administered subcutaneously to the subject in need thereof. In another example, the formulation of the disclosure is administered intravenously to the subject in need thereof.

In one example, the formulation of the disclosure is self-administered.

In one example, the formulation of the disclosure is self-administered subcutaneously.

In one example, the formulation of the disclosure is provided in a pre-filled syringe.

In one example, the formulation of the disclosure is self-administered subcutaneously, with a pre-filled syringe.

In one example of any method described herein, the subject is a mammal, for example a primate such as a human.

Methods of treatment described herein can additionally comprise administering a further compound to reduce, treat or prevent the effect of the thrombotic disorder, inflammatory disorder and/or thrombo-inflammatory disorder.

The present disclosure provides a kit comprising at least one pharmaceutical formulation of the present disclosure packed with instructions for use in antagonising activity and/or antagonising activation of Factor XII and/or an activated form thereof in a subject. Optionally, the kit additionally comprises a further therapeutically active compound or drug.

The present disclosure further provides a kit comprising at least one pharmaceutical formulation of the present disclosure packaged with instructions for use in treating or preventing a disorder in a subject. Optionally, the kit additionally comprises a further therapeutically active compound or drug.

The present disclosure also provides a kit comprising at least one pharmaceutical formulation of the present disclosure packaged with instructions to administer the conjugate or composition to a subject who is suffering from or at risk of suffering from a disorder, optionally, in combination with a further therapeutically active compound or drug.

In one example, the formulation is present in a vial, syringe or an autoinjector device. For example, the syringe is a pre-filled syringe.

The present disclosure provides a vial comprising a pharmaceutical formulation of the present disclosure. For example, the vial is a single use vial.

The present disclosure provides a syringe comprising a pharmaceutical formulation of the present disclosure.

The present disclosure also provides an autoinjector device comprising a pharmaceutical formulation of the present disclosure.

The present disclosure provides a prefilled syringe comprising pharmaceutical formulation comprising about 170 mg/ml of an antibody or antigen binding fragment thereof described herein, a histidine buffer having a pH of 5.5 to 6.5, polysorbate 80 and proline and arginine monohydrochloride as stabilisers, wherein the formulation has a viscosity of less than about 10 mPa*s at 20° C. and 25° C. For example, the prefilled syringe comprises a formulation comprising 12 mM to-25 mM histidine buffer having a pH of 5.8 to 6.4, 0.01% to 0.03% (w/v) polysorbate 80, 110 mM to 170 mM proline and 110 mM to 170 mM arginine. In one example, the osmolality of the formulation is about 450 mOsm/kg. In one example, the antibody comprises a V_{H} comprising an amino acid sequence set forth in SEQ ID NO: 5 and a V_{L} comprising an amino acid sequence set forth in SEQ ID NO: 6. In one example, the volume of the prefilled syringe is between 0.5 ml and 2 ml.

The present disclosure provides a prefilled syringe comprising a pharmaceutical formulation comprising between about 160 mg/ml to 180 mg/ml of an antibody or antigen binding fragment thereof described herein, a histidine buffer having a pH of 5.8 to 6.4, polysorbate 80, proline and arginine monohydrochloride as stabilisers, wherein the formulation has a viscosity of less than about 10 mPa*s at 20° C. and 25° C. For example, the prefilled syringe comprises a formulation comprising between 12 to 25 mM histidine buffer having a pH of 5.8 to 6.4, 0.01% to 0.03% (w/v) polysorbate 80, 90 mM to 150 mM proline and 100 mM to 160 mM arginine. In one example, the osmolality of the formulation is between about 430 to 530 mOsm/kg. In one example, the antibody comprises a V_{H} comprising an amino acid sequence set forth in SEQ ID NO: 5 and a V_{L} comprising an amino acid sequence set forth in SEQ ID NO: 6. In one example, the volume of the prefilled syringe is between 0.5 ml and 2 ml.

The present disclosure provides a prefilled syringe comprising a pharmaceutical formulation comprising about 170 mg/ml of an antibody or antigen binding fragment thereof described herein, a histidine buffer having a pH of 5.5 to 6.5, polysorbate 80 and proline and arginine monohydrochloride as stabilisers, wherein the formulation has a viscosity of less than about 10 mPa*s at 20° C. and 25° C. For example, the prefilled syringe comprises a formulation comprising about 20 mM histidine buffer having a pH of 5.8 to 6.4, 0.02% (w/v) polysorbate 80, 140 mM proline and 150 mM arginine. In one example, the osmolality of the formulation is about 450 mOsm/kg. In one example, the antibody comprises a V_{H} comprising an amino acid sequence set forth in SEQ ID NO: 5 and a V_{L} comprising an amino acid sequence set forth in SEQ ID NO: 6. In one example, the volume of the prefilled syringe is between 0.5 ml and 2 ml.

The present disclosure provides a vial comprising a pharmaceutical formulation comprising about 100 mg/ml of an antibody or antigen binding fragment thereof described herein, a histidine buffer having a pH of 5.5 to 6.5, polysorbate 80 and proline and arginine monohydrochloride as stabilisers, wherein the formulation has a viscosity of less than about 10 mPa*s at 20° C. and 25° C. For example, the vial comprises a formulation comprises between 12-25 mM histidine buffer having a pH of 5.8 to 6.4, 0.01% to 0.03% (w/v) polysorbate 80, 90 mM to 150 mM proline and 100 mM to 160 mM arginine. In one example, the osmolality of the formulation is about 450 mOsm/kg. In one example, the antibody comprises a V_{H} comprising an amino acid sequence set forth in SEQ ID NO: 5 and a V_{L} comprising an amino acid sequence set forth in SEQ ID NO: 6. In one example, the vial has a volume of 2 ml.

The present disclosure provides a vial comprising a pharmaceutical formulation comprising about 100 mg/ml of an antibody or antigen binding fragment thereof described herein, a histidine buffer having a pH of 5.5 to 6.5, polysorbate 80 and proline and arginine as stabilisers, wherein the formulation has a viscosity of less than about 5 mPa*s at 20° C. and 25° C. For example, the vial comprises a formulation comprises about 20 mM histidine buffer having a pH of 5.8 to 6.4, 0.02% (w/v) polysorbate 80, 140 mM proline and 150 mM arginine. In one example, the osmolality of the formulation is about 450 mOsm/kg. In one example, the osmolality of the formulation is about 450 mOsm/kg. In one example, the antibody comprises a V_{H} comprising an amino acid sequence set forth in SEQ ID NO: 5 and a V_{L} comprising an amino acid sequence set forth in SEQ ID NO: 6. In one example, the vial has a volume of 2 ml.

Exemplary effects of the pharmaceutical formulation of the present disclosure are described herein and are to be taken to apply mutatis mutandis to the examples of the disclosure set out in the previous paragraphs.

### KEY TO SEQUENCE LISTING

SEQ ID NO: 1 amino acid sequence of 3F7 V_{H}
SEQ ID NO: 2 amino acid sequence of 3F7 V_{L}
SEQ ID NO: 3 amino acid sequence of 3F7G V_{H}
SEQ ID NO: 4 amino acid sequence of 3F7G V_{L}
SEQ ID NO: 5 amino acid sequence of affinity matured 3F7 V_{H}
SEQ ID NO: 6 amino acid sequence of affinity matured 3F7 V_{L}
SEQ ID NO: 7 amino acid sequence of 3F7 V_{H} CDR1
SEQ ID NO: 8 amino acid sequence of 3F7 V_{H} CDR2
SEQ ID NO: 9 amino acid sequence of 3F7 V_{H} CDR3
SEQ ID NO: 10 amino acid consensus sequence of 3F7 variant V_{H} CDR2
SEQ ID NO: 11 amino acid consensus sequence of 3F7 variant V_{H} CDR3
SEQ ID NO: 12 amino acid sequence of 3F7 V_{L} CDR1
SEQ ID NO: 13 amino acid sequence of 3F7 V_{L} CDR2
SEQ ID NO: 14 amino acid sequence of 3F7 V_{L} CDR3
SEQ ID NO: 15 amino acid consensus sequence of 3F7 variant V_{L} CDR3
SEQ ID NO: 16 amino acid sequence of affinity matured 3F7 V_{H} CDR2
SEQ ID NO: 17 amino acid sequence from a human Factor XII

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, antibodies and fragments thereof herein may be further clarified by the discussion in Kabat Sequences of Proteins of Immunological Interest, National Institutes of Health, Bethesda, Md., 1987 and 1991.

The term “EU numbering system of Kabat” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

Coagulation Factor XII, also known as Hageman factor or FXII, is a plasma protein. It is the zymogen form of Factor XIIa, an enzyme of the serine protease (or serine endopeptidase) class. In humans, Factor XII is encoded by the F12 gene. For the purposes of nomenclature only and not limitation exemplary sequences of human Factor XII is set out in NCBI Reference Sequence: NP_000496.2; in NCPI protein accession number NP_000496 and in SEQ ID NO: 17. Additional sequences of Factor XII can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)).

As used herein, the term “Factor XII inhibitor” or “FXII inhibitor” or “inhibitor of FXII” refers to an inhibitor of either or both of Factor XII (prior to activation, i.e., its zymogen) and activated Factor XII (FXIIa) as well as to the activation of FXII. Thus, “inhibitor(s) of FXII” can include inhibitors of either or both of FXII and FXIIa (also termed αFXIIa) as well as the activation of FXII, including the FXIIa cleavage products FXIIa alpha and FXIIa beta (also termed FXIIf). FXII inhibitors encompass functional variants and fragments of the wild-type inhibitor. A functional variant or fragment is a molecule that retains at least 50% (e.g., about 50%, or about 60%, or about 70%, or about 80%, or about 90%, or about 95%, or about 99%, or about 100%) of the ability of the wild-type molecule to inhibit FXII, FXIIa or the activation of FXII. In one example, the FXII inhibitors are non-endogenous inhibitors; that is, they are not inhibitors that occur naturally in the human or animal body.

The term “amidolytic activity” refers to the ability of the protein of the present disclosure to catalyse the hydrolysis of at least one peptide bond in another polypeptide. The term “organic acid buffer” refers to conventional buffers of organic acids and salts.

The term “non-ionic surfactant” as used herein refers to any detergent that has an uncharged polar head.

A “stable” formulation is one in which the antibody or antigen binding fragment thereof essentially retains its physical stability and/or chemical stability and/or biological activity upon storage.

In the context of the present disclosure, the term “monomer” or “monomeric” refers to the correctly folded antibody or antigen binding fragment thereof. For example, a monomer of an antibody according to the present disclosure relates to the standard tetrameric antibody comprising two identical, glycosylated heavy and light chains respectively. An “aggregate” is then a non-specific association of two antibody molecules (e.g., high molecular weight species).

As used herein, the term “amino acid stabiliser” refers to an amino acid or derivative thereof that improves or otherwise enhances the stability of the formulation.

As used herein, the term “polyol” refers to a substance having a plurality of hydroxyl groups.

The term “dynamic viscosity” or “absolute viscosity” refers to the internal resistance to flow exhibited by a fluid at a specified temperature (e.g., 20° C.), the ratio of shearing stress to rate of shear. A liquid has a dynamic viscosity of one poise if a force of 1 dyne/square centimetre causes two parallel liquid surfaces one square centimetre in area and one square centimetre apart to move past one another at a velocity of 1 cm/second. One poise equals one hundred centipoise (cP) and one centipoise equals one millipascal-second (mPa*s) in System International (SI) units.

As used herein, the term “density” of a formulation refers to the volumetric mass density or the mass per unit volume (g/cm³).

As used herein, the term “osmolality” is a measure of the osmoles (Osm) of solute per kilogram of solvent (osmol/kg or Osm/kg).

As used herein, the term “binds” in reference to the interaction of an antibody or an antigen binding fragment thereof with an antigen means that the interaction is dependent upon the presence of a particular structure (e.g., an antigenic determinant or epitope) on the antigen. For example, an antibody, or antigen binding fragment thereof, recognises and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antibody or antigen binding fragment thereof reacts or associates more frequently, more rapidly, with greater duration and/or with greater affinity with a particular antigen or cell expressing same than it does with alternative antigens or cells. For example, an antibody or an antigen binding fragment thereof binds to FXII (or FXIIa) with materially greater affinity (e.g., 1.5 fold or 2 fold or 5 fold or 10 fold or 20 fold or 40 fold or 60 fold or 80 fold to 100 fold or 150 fold or 200 fold) than it does to other blood clotting factors or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

The term “recombinant” shall be understood to mean the product of artificial genetic recombination. Accordingly, in the context of an antibody or antigen binding fragment thereof, this term does not encompass an antibody naturally occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

For the purposes of the present disclosure, the term “antibody” includes a protein capable of specifically binding to one or a few closely related antigens (e.g., a blood coagulation factor) by virtue of an antigen binding domain contained within a Fv. This term includes four chain antibodies (e.g., two light chains and two heavy chains), recombinant or modified antibodies (e.g., chimeric antibodies, humanised antibodies, human antibodies, CDR-grafted antibodies, primatised antibodies, de-immunised antibodies, synhumanised antibodies, half-antibodies, bispecific antibodies). Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (such as, human) antibody. In one example the antibody heavy chain is missing a C-terminal lysine residue. In one example, the antibody is humanised, synhumanised, chimeric, CDR-grafted or deimmunised.

An “anti-FXII antibody” includes antibodies that bind to and/or inhibit either or both of the zymogen of FXII and the activated protein (FXIIa), including the FXIIa alpha and FXIIa beta cleavage fragments. In some examples, the antibody binds specifically to FXIIa or the alpha or beta chain fragments of FXIIa.

As used herein the term “germlined” antibody refers to an antibody where some or all somatic mutations that introduced changes into the framework residues are reversed to the original sequence present in the genome, e.g., a human genome. In this regard, not all changes need to be reversed in a germlined antibody.

As used herein, “variable region” refers to the portions of the light and/or heavy chains of an antibody as defined herein that is capable of specifically binding to an antigen and, includes amino acid sequences of complementarity determining regions (CDRs); i.e., CDRI, CDR2, and CDR3, and framework regions (FRs).

As used herein, the term “complementarity determining regions” (syn. CDRs; i.e., CDRI, CDR2, and CDR3) refers to the amino acid residues of an antibody variable region the presence of which are major contributors to specific antigen binding. Each variable region typically has three CDR regions identified as CDRI, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”. According to the numbering system of Kabat, V_{H} FRs and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4).

“Framework regions” (hereinafter FR) are those variable domain residues other than the CDR residues.

An “antigen binding fragment” of an antibody comprises one or more variable regions of an intact antibody. Examples of antibody fragments include Fab, Fab′, F(ab′)₂ and Fv fragments; diabodies; linear antibodies; single-chain antibody molecules, half antibodies and multispecific antibodies formed from antibody fragments.

As used herein, the term “Fv” shall be taken to mean any protein, whether comprised of multiple polypeptides or a single polypeptide, in which a variable region of the light chain (V_{L}) and a variable region of a heavy chain (V_{H}) associate and form a complex having an antigen binding domain, i.e., capable of specifically binding to an antigen. The V_{H} and the V_{L} which form the antigen binding domain can be in a single polypeptide chain or in different polypeptide chains. Furthermore, an Fv of the disclosure (as well as any protein of the disclosure) may have multiple antigen binding domains which may or may not bind the same antigen. This term shall be understood to encompass fragments directly derived from an antibody as well as proteins corresponding to such a fragment produced using recombinant means. Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “fragment crystallisable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG₁), gamma 2 (IgG₂), gamma 3 (IgG₃) and gamma 4 (IgG₄), or hybrids thereof.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy (C_{H}) C_{H}1, a linker, a C_{H}2 and a C_{H}3. In a heavy chain, a constant region comprises a Fc. In a light chain, a constant region generally comprises one constant domain (a C_{L}1).

The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognising two distinct antigens (resulting in bispecific molecules). Fab arm exchange occurs naturally in vivo and can be induced in vitro by purified blood cells or reducing agents such as reduced glutathione.

As used herein, the term “monospecific” refers to a binding domain comprising one or more antigen binding sites each with the same epitope specificity. Thus, a monospecific binding domain can comprise a single antigen binding site (e.g., a Fv, scFv, Fab, etc) or can comprise several antigen binding sites that recognise the same epitope (e.g., are identical to one another), e.g., a diabody or an antibody. The requirement that the binding region is “monospecific” does not mean that it binds to only one antigen, since multiple antigens can have shared or highly similar epitopes that can be bound by a single antigen binding site. A monospecific binding domain that binds to only one antigen is said to “exclusively bind” to that antigen.

The term “multispecific” refers to a binding domain comprising two or more antigen binding sites, each of which binds to a distinct epitope, for example each of which binds to a distinct antigen. For example, the multispecific binding domain may include antigen binding sites that recognise two or more different epitopes of the same protein (e.g., coagulation factor) or that may recognise two or more different epitopes of different proteins (i.e., distinct coagulation factors). In one example, the binding domain may be “bispecific”, that is, it includes two antigen binding sites that specifically bind two distinct epitopes. For example, a bispecific binding domain specifically binds or has specificities for two different epitopes on the same protein. In another example, a bispecific binding domain specifically binds two distinct epitopes on two different proteins.

As used herein, the term “binds” in reference to the interaction of a compound or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a conjugate comprising an antibody Fc that binds to Factor XII (e.g., human Factor XII) with materially greater affinity (e.g., 20 fold or 40 fold or 60 fold or 80 fold to 100 fold or 150 fold or 200 fold) than it does to other cytokine receptor or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

The term “competitively inhibits” shall be understood to mean that a protein of the disclosure (or an antigen binding site thereof) reduces or prevents binding of a recited antibody or protein to Factor XII and/or Factor XIIa. This may be due to the protein (or antigen binding site) and antibody binding to the same or an overlapping epitope. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Preferably, the protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to Factor XII either in the presence or absence of the protein. If less antibody binds in the presence of the protein than in the absence of the protein, the protein is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit a protein (or antigen binding site thereof) that binds to one epitope to competitively inhibit the binding of a protein (or antigen binding site) that binds to the other epitope. For example, the “overlapping” epitopes share at least 1 or 2 or 3 or 4 or 5 or 6 or 7 or 8 or 9 or 10 amino acids.

The phrase “conservative amino acid substitution” refers to replacement or substitution of an amino acid residue with an amino acid residue having a similar side chain and/or hydropathicity and/or hydrophilicity. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophilic indices are described in, e.g., U.S. Pat. No. 4,554,101.

As used herein, the terms “disease”, “disorder” or “condition” refers to a disruption of or interference with normal function, and is not to be limited to any specific condition, and will include diseases or disorders.

As used herein, the term “thrombotic disorder” refers to a condition which is characterised by the development of blood clots, or the potential to develop blood clots. Blood clots are also referred to as “thrombi”, “thrombus” or “thrombosis”.

As used herein, the term “inflammatory disorder” refers to a condition which is characterised by the development of an inflammation, or the potential to develop inflammation.

As used herein, the term “thrombo-inflammatory disorder” refers to any condition which is characterised as having both inflammatory and thrombotic tendencies.

As used herein, the phrase “thrombotic disorder, inflammatory disorder and/or thrombo-inflammatory disorder” refers to diseases or conditions which are classified as one or more of a thrombotic disorder, inflammatory disorder or thrombo-inflammatory disorder.

As used herein, the terms “treating”, “treat” or “treatment” include administering a protein described herein to thereby reduce or eliminate at least one symptom of a specified disease or condition or to slow progression of the disease or condition.

As used herein, the terms “preventing”, “prevent” or “prevention” includes providing prophylaxis with respect to occurrence or recurrence of a specified disease or condition in an individual. An individual may be predisposed to or at risk of developing the disease or disease relapse but has not yet been diagnosed with the disease or the relapse.

As used herein, a subject “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change in a factor associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the antibody or antigen binding fragment thereof are outweighed by the therapeutically beneficial effects.

As used herein, the term “subject” shall be taken to mean any animal including humans, for example a mammal. Exemplary subjects include but are not limited to humans and non-human primates. For example, the subject is a human.

### Proteins of the Pharmaceutical Formulation

As discussed herein, the present disclosure provides a liquid pharmaceutical formulation comprising at least 100 mg/ml of a protein comprising an antigen binding domain that binds to or specifically binds to Factor XII and/or an activated form thereof (i.e., activated FXII; FXIIa).

### Proteins Comprising Antigen Binding Domains

The present disclosure provides a pharmaceutical formulation comprising a protein comprising an antigen binding domain that binds to or specifically binds to Factor XII and/or an activated form thereof. For example, the protein comprises at least a V_{H} and a V_{L}, wherein the V_{H} and V_{L} bind to form a Fv comprising an antigen binding domain.

In one example, the antigen binding domain binds to or specifically binds to Factor XII and/or an activated form thereof and antagonises the activity of Factor XII and/or activated Factor XII. For example, the protein binds to or specifically binds to Factor XII and antagonises Factor XII activity. In another example, the protein binds to or specifically binds to activated Factor XII (FXIIa) and antagonises Factor XIIa activity.

In one example, the antigen binding domain binds to or specifically binds to Factor XII and/or an activated form thereof and antagonises the activation of Factor XII and/or activated Factor XII. For example, the protein binds to or specifically binds to Factor XII and inhibits the activation of Factor XII to Factor XIIa.

### Antibodies or Antigen Binding Fragments

In one example, the protein comprising an antigen binding domain that binds to or specifically binds to Factor XII and/or an activated form thereof is an antibody or antigen binding fragment. For example, the protein is an antibody or antigen binding fragment that binds to Factor XII and/or activated Factor XII (FXIIa). For example, the protein is an antibody or antigen binding fragment that binds to Factor XII. In another example, the protein is an antibody or antigen binding fragment that binds to activated Factor XII (FXIIa).

Methods for generating antibodies are known in the art and/or described in Harlow and Lane (editors) Antibodies: A Laboratory Manual, Cold Spring Harbor Laboratory, (1988). Generally, in such methods Factor XII (e.g., hFXII) or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present disclosure may be a synthetic antibody. For example, the antibody is a chimeric antibody, a humanised antibody, a human antibody or a de-immunised antibody.

The antibodies or antigen binding fragments of the present disclosure may be humanised.

The term “humanised antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanised antibodies also include antibodies in which one or more residues of the human protein are modified by one or more amino acid substitutions and/or one or more FR residues of the human antibody are replaced by corresponding non-human residues. Humanised antibodies may also comprise residues which are found in neither the human antibody nor in the non-human antibody. Any additional regions of the antibody (e.g., Fc region) are generally human. Humanisation can be performed using a method known in the art, e.g., U.S. Pat. No. 5,225,539, 6,054,297, 7,566,771 or 5,585,089. The term “humanised antibody” also encompasses a super-humanised antibody, e.g., as described in U.S. Pat. No. 7,732,578. A similar meaning will be taken to apply to the term “humanised antigen binding fragment”.

The antibodies or antigen binding fragments thereof of the present disclosure may be human antibodies or antigen binding fragments thereof. The term “human antibody” as used herein refers to antibodies having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human antibody will also be considered to include a protein comprising FRs from a human antibody or FRs comprising sequences from a consensus sequence of human FRs and in which one or more of the CDRs are random or semi-random, e.g., as described in U.S. Pat. Nos. 6,300,064 and/or 6,248,516. A similar meaning will be taken to apply to the term “human antigen binding fragment”.

The antibodies or antigen binding fragments thereof of the present disclosure may be synhumanised antibodies or antigen binding fragments thereof. The term “synhumanised antibody” refers to an antibody prepared by a method described in WO2007019620. A synhumanised antibody includes a variable region of an antibody, wherein the variable region comprises FRs from a New World primate antibody variable region and CDRs from a non-New World primate antibody variable region.

The antibody or antigen binding fragment thereof of the present disclosure may be primatised. A “primatised antibody” comprises variable region(s) from an antibody generated following immunisation of a non-human primate (e.g., a cynomolgus macaque). Optionally, the variable regions of the non-human primate antibody are linked to human constant regions to produce a primatised antibody. Exemplary methods for producing primatised antibodies are described in U.S. Pat. No. 6,113,898.

In one example an antibody or antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a V_{H} and/or a V_{L} from a non-human antibody (e.g., a murine antibody) and the remaining regions of the antibody are from a human antibody. The production of such chimeric antibodies and antigen binding fragments thereof is known in the art, and may be achieved by standard means (as described, e.g., in U.S. Pat. Nos. 6,331,415; 5,807,715; 4,816,567 and 4,816,397).

The present disclosure also contemplates a deimmunised antibody or antigen binding fragment thereof, e.g., as described in WO2000034317 and WO2004108158. De-immunised antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analysed to identify one or more B or T cell epitopes and one or more amino acid residues within the epitope is mutated to thereby reduce the immunogenicity of the antibody.

Exemplary human antibodies are described herein and include 3F7, 3F7G and affinity matured 3F7 and/or variable regions thereof. A further exemplary antibody is the anti-FXII antibody garadacimab. These human antibodies provide an advantage of reduced immunogenicity in a human compared to non-human antibodies. Exemplary antibodies are described in WO 2013/014092 and WO 2017/173494, which are incorporated herein by reference. Additional antibodies and proteins comprising variable regions are described in WO 2006/066878, and in Ravon et al., Blood 86: 4134-43 (1995).

### Bispecific Antibodies

In one example, the protein of the present disclosure may be a bispecific antibody or fragment thereof. For example, the antibody or fragment may bind to Factor XII and/or an activated form thereof, and another target. A bispecific antibody is a molecule comprising two types of antibodies or antibody fragments (e.g., two half antibodies) having specificities for different antigens or epitopes. Exemplary bispecific antibodies bind to two different epitopes of the same protein. Alternatively, the bispecific antibody binds to two different epitopes on two different proteins. Exemplary “key and hole” or “knob and hole” bispecific proteins as described in U.S. Pat. No. 5,731,168. In one example, a constant region (e.g., an IgG₄ constant region) comprises a T366W mutation (or knob) and a constant region (e.g., an IgG₄ constant region) comprises a T366S, L368A and Y407V mutation (or hole). In another example, the first constant region comprises T350V, T366L, K392L and T394W mutations (knob) and the second constant region comprises T350V, L351Y, F405A and Y407V mutations (hole).

Methods for generating bispecific antibodies are known in the art and exemplary methods are described herein.

In one example, an IgG type bispecific antibody is secreted by a hybrid hybridoma (quadroma) formed by fusing two types of hybridomas that produce IgG antibodies (Milstein C et al., Nature 1983, 305: 537-540). In another example, the antibody can be secreted by introducing into cells genes of the L chains and H chains that constitute the two IgGs of interest for co-expression (Ridgway, J B et al. Protein Engineering 1996, 9: 617-621; Merchant, A M et al. Nature Biotechnology 1998, 16: 677-681).

In one example, a bispecific antibody fragment is prepared by chemically cross-linking Fab's derived from different antibodies (Keler T et al. Cancer Research 1997, 57: 4008-4014).

In one example, a leucine zipper derived from Fos and Jun or the like is used to form a bispecific antibody fragment (Kostelny S A et al. J. of Immunology, 1992, 148: 1547-53).

In one example, a bispecific antibody fragment is prepared in a form of diabody comprising two crossover scFv fragments (Holliger P et al. Proc. of the National Academy of Sciences of the USA 1993, 90: 6444-6448).

### Antibody Fragments

As described herein, a protein of the disclosure comprises an antigen binding fragment linked to a constant region of an antibody, Fc or a heavy chain constant domain C_{H}2 and/or C_{H}3. Exemplary antigen binding fragments for use in the present disclosure are described below.

### Single-Domain Antibodies

In some examples, an antigen binding fragment of an antibody of the disclosure is or comprises a single-domain antibody (which is used interchangeably with the term “domain antibody” or “dAb”). A single-domain antibody is a single polypeptide chain comprising all or a portion of the heavy chain variable domain of an antibody.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favoured linkers for a scFv.

In one example, the linker comprises the sequence SGGGGSGGGGSGGGGS.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

In some examples, an antigen binding fragment of the disclosure is or comprises a heavy chain antibody. Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR). A general description of heavy chain antibodies from camelids and the variable regions thereof and methods for their production and/or isolation and/or use is found inter alia in the following references WO 94/04678, WO 97/49805 and WO 97/49805. A general description of heavy chain antibodies from cartilaginous fish and the variable regions thereof and methods for their production and/or isolation and/or use is found inter alia in WO 2005/118629.

### Half-Antibodies

In some examples, the antigen binding fragment of the present disclosure is a half-antibody or a half-molecule. The skilled artisan will be aware that a half antibody refers to a protein comprising a single heavy chain and a single light chain. The term “half antibody” also encompasses a protein comprising an antibody light chain and an antibody heavy chain, wherein the antibody heavy chain has been mutated to prevent association with another antibody heavy chain. In one example, a half antibody forms when an antibody dissociates to form two molecules each containing a single heavy chain and a single light chain.

Methods for generating half antibodies are known in the art and exemplary methods are described herein.

In one example, the half antibody can be secreted by introducing into cells genes of the single heavy chain and single light chain that constitute the IgG of interest for expression. In one example, a constant region (e.g., an IgG₄ constant region) comprises a “key or hole” (or “knob or hole”) mutation to prevent heterodimer formation. In one example, a constant region (e.g., an IgG₄ constant region) comprises a T366W mutation (or knob). In another example, a constant region (e.g., an IgG₄ constant region) comprises a T366S, L368A and Y407V mutation (or hole). In another example, the constant region comprises T350V, T366L, K392L and T394W mutations (knob). In another example, the constant region comprises T350V, L351Y, F405A and Y407V mutations (hole). Exemplary constant region amino acid substitutions are numbered according to the EU numbering system.

### Other Antibodies and Antibody Fragments

The present disclosure also contemplates other antibodies and antibody fragments, such as:
(i) minibodies, e.g., as described in U.S. Pat. No. 5,837,821;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### Stabilised Proteins

Proteins of the present disclosure can comprise an IgG₄ constant region or a stabilized IgG₄ constant region. The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognizing two distinct antigens (resulting in bispecific molecules). Fab arm exchange occurs naturally in vivo and can be induced in vitro by purified blood cells or reducing agents such as reduced glutathione.

In one example, a stabilised IgG₄ constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG₄, this residue is generally a serine. Following substitution of the serine for proline, the IgG₄ hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG₁ according to the numbering system of Kabat. Hinge regions of other IgG isotypes may be aligned with the IgG₁ sequence by placing the first and last cysteine residues forming inter-heavy chain disulphide (S—S) bonds in the same positions (see for example WO2010080538).

### Preparation of the Pharmaceutical Formulation

The present disclosure provides a liquid pharmaceutical formulation comprising at least 100 mg/ml of a protein comprising an antigen binding domain that binds to or specifically binds to Factor XII and/or an activated form thereof (i.e., activated FXII; FXIIa), an organic acid buffer, a non-ionic surfactant and an amino acid stabiliser, wherein the formulation has a pH of 5.0 to 6.5 and a viscosity of less than about 30 mPa*s at 20° C.

Preparation of the pharmaceutical formulation is performed according to standard methods known in the art and/or according to methods described herein.

### Organic Acid Buffers

In one example, the present disclosure provides a pharmaceutical formulation comprising at least 100 mg/ml of a protein of the disclosure and an organic acid buffer having a pH of 5.0 to 6.5.

The skilled person will understand that organic acid buffers suitable for use in the present disclosure comprise one or more carboxylic acid or acid phenolic groups without basic amino groups. In addition to the buffering capacity provided by the acidic groups, such organic buffers used herein can contain additional ionisable functionality provided by, for example, an amino group.

It will be apparent to the skilled person that buffers suitable for use in the present disclosure will be stable and effective at the desired pH and will provide sufficient buffer capacity to maintain the desired pH over the range of conditions to which it will be exposed during formulation and storage of the product. For example, a stable buffer will provide thermal aggregation stability (e.g., during freeze/thaw or elevated temperatures), not be affected by oxidation of physical degradation (e.g., insoluble particulate formation) and provide the desired polydispersity (i.e., particle distribution). Suitable buffers will not form deleterious complexes with metal ions, be toxic, or unduly penetrate, solubilise, or absorb on membranes or other surfaces. Furthermore, the skilled person will recognise that such buffers should not interact with other components of the composition in any manner which decreases their availability or effectiveness. Additionally, the buffering agent of the pharmaceutical formulation must be safe for administration, compatible with other components of the composition over the shelf-life of the product, and acceptable for administration to the subject.

Suitable organic acid buffers for use in the present disclosure will be apparent to the skilled person and include, for example, histidine buffers (e.g., histidine chloride, histidine acetate, histidine phosphate, histidine sulfate, etc.), glutamate buffers (e.g., monosodium glutamate, etc.), citrate buffers (e.g. monosodium citrate-disodium citrate mixture, citric acid-trisodium citrate mixture, citric acid-monosodium citrate mixture, etc.), succinate buffers (e.g. succinic acid-monosodium succinate mixture, succinic acid-sodium hydroxide mixture, succinic acid-disodium succinate mixture, etc.), tartrate buffers (e.g. tartaric acid-sodium tartrate mixture, tartaric acid-potassium tartrate mixture, tartaric acid-sodium hydroxide mixture, etc.), fumarate buffers (e.g. fumaric acid-monosodium fumarate mixture, fumaric acid-disodium fumarate mixture, monosodium fumarate-disodium fumarate mixture, etc.) gluconate buffers (e.g. gluconic acid-sodium gluconate mixture, gluconic acid-sodium hydroxide mixture, gluconic acid-potassium gluconate mixture, etc.), oxalate buffers (e.g. oxalic acid-sodium oxalate mixture, oxalic acid-sodium hydroxide mixture, oxalic acid-potassium oxalate mixture, etc.), lactate buffers (e.g. lactic acid-sodium lactate mixture, lactic acid-sodium hydroxide mixture, lactic acid-potassium lactate mixture, etc.) and acetate buffers (e.g. acetic acid-sodium acetate mixture, acetic acid-sodium hydroxide mixture, etc.).

In one example of the present disclosure, the organic acid buffer is selected from the group consisting of a histidine buffer, a glutamate buffer, a succinate buffer and a citrate buffer. For example, the organic acid buffer is a glutamate buffer. For example, the organic acid buffer is a histidine buffer. For example, the organic acid buffer is L-histidine.

Methods of assessing the suitability of buffers will be apparent to the skilled person and/or described herein and include, for example, differential scanning fluorimetry and dynamic light scattering.

### Non-Ionic Surfactants

In one example, the present disclosure provides a pharmaceutical formulation comprising at least 100 mg/ml of a protein of the disclosure and a non-ionic surfactant.

The amount of surfactant added to the pharmaceutical formulation will be apparent to the skilled person and is in an amount such that it suppresses aggregation (e.g., by preventing surface denaturation), increases stabilisation (e.g., during thermal and/or physical stress), minimises the formation of particulates in the formulation (e.g., sub-visible particle formation), reduces surface adsorption and/or assists in protein refolding.

Suitable non-ionic surfactants for use in the present disclosure will be apparent to the skilled person and include, for example, polyoxyethylensorbitan fatty acid esters (e.g., polysorbate 20 and polysorbate 80), polyethylene-polypropylene copolymers, polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS).

In one example of the present disclosure, the non-ionic surfactant is selected from the group consisting of polyoxyethylensorbitan fatty acid esters and polyoxyethylene-polyoxypropylene copolymers. For example, the polyoxyethylensorbitan fatty acid ester is polyoxyethylene sorbitan monooleate (i.e., polysorbate 80) or polyoxyethylene sorbitan monolaurate (polysorbate 20).

### Amino Acid Stabilisers

In one example, the present disclosure provides a pharmaceutical formulation comprising at least 100 mg/ml of a protein of the present disclosure and an amino acid stabiliser.

The amount of amino acid stabiliser added to the pharmaceutical formulation will be apparent to the skilled person and is in an amount that such that it reduces thermal and/or physical stress (e.g., freeze/thaw or agitation), and/or confers or enhances stability of the protein.

Suitable amino acids for use in the present disclosure will be apparent to the skilled person and include, for example, glycine, alanine, valine, leucine, isoleucine, methionine, threonine, phenylalanine, tyrosine, serine, cysteine, histidine, tryptophan, proline, aspartic acid, glutamic acid, arginine, lysine, ornithine and asparagine and salts thereof.

In one example of the present disclosure, the amino acid is selected from the group consisting of proline, arginine and methionine. For example, the amino acid stabiliser is proline or a salt form thereof. For example, the amino acid stabiliser is arginine or a salt form thereof. For example, the amino acid stabilisers are proline and arginine or a salt form thereof.

### Polyols

In one example, the present disclosure provides a pharmaceutical formulation comprising at least 100 mg/ml of a protein of the disclosure and a polyol.

The amount of polyol added to the pharmaceutical formulation will be apparent to the skilled person and is in an amount that is effective in reducing aggregation and increase stabilisation of the antibody or fragment thereof. For example, the polyol for use in the present disclosure assist in maintaining the antibody or fragment thereof in a compact state (e.g., reduce unfolding and/or aggregation).

Suitable polyols for use in the present disclosure will be apparent to the skilled person and include, for example, a sugar (reducing or non-reducing sugar), a sugar alcohol and saccharic acid. “Reducing sugar” means a sugar capable of reducing metal ions or a sugar containing a hemiacetal group capable of reacting covalently with lysine within a protein and other amino groups. Examples of a reducing sugar include, for example, fructose, mannose, maltose, lactose, arabinose, xylose, ribose, rhamnose, galactose, and glucose. “Non-reducing sugar” has no such properties. Examples of a non-reducing sugar include, for example, sucrose, trehalose, sorbose, melezitose, and raffinose. Examples of a sugar alcohol include mannitol, xylitol, erythritol, threitol, sorbitol, and glycerol. In one example of the present disclosure, the polyol is sorbitol. Examples of a saccharic acid include L-gluconic acid and its metal salt.

### Assaying the Pharmaceutical Formulation and Proteins of the Disclosure

High concentration pharmaceutical formulations and proteins of the present disclosure are readily screened for physical and biological activity and/or stability using methods known in the art and/or as described below.

Binding to Factor XII and/or Factor XIIa

It will be apparent to the skilled artisan from the disclosure herein that a protein of the present disclosure binds (or specifically binds) to the ligand binding domain of Factor XII and/or activated Factor XII (i.e., Factor XIIa). Methods for assessing binding to a protein are known in the art, e.g., as described in Scopes (In: Protein purification: principles and practice, Third Edition, Springer Verlag, 1994). Such a method generally involves labelling the protein and contacting it with immobilised compound. Following washing to remove non-specific bound protein, the amount of label and, as a consequence, bound protein is detected. Of course, the protein can be immobilised and the compound that binds to Factor XII and/or Factor XIIa labelled. Panning-type assays can also be used. Alternatively, or additionally, surface plasmon resonance assays can be used.

The assays described above can also be used to detect the level of binding of a protein of the present disclosure to Factor XII and/or Factor XIIa or a ligand binding domain thereof. Methods of detecting the level of binding will be apparent to the skilled person and/or described herein. For example, the level of binding is determined using a biosensor.

### Measuring Factor XII/XIIa Activity

Methods for assessing the antagonistic activity of a protein are known in the art, and include for example a chromogenic assay. Chromogenic assays for measuring inhibitory activity are known in the art (e.g., Chromogenix S-2302™; Diapharma).

In one example, assay buffer is pre-mixed with Factor XIIa. The conjugate of the present disclosure is added followed by chromogenic substrate. Following cessation of the chromogenic reaction, the inhibitory activity of the conjugate is assessed.

### Determining Competitive Binding

Assays for determining a protein that competitively inhibits binding of antibodies 3F7 and/or 3F7G (or any other antibody described herein) will be apparent to the skilled artisan. For example, 3F7 or 3F7G is conjugated to a detectable label, e.g., a fluorescent label or a radioactive label. The labelled antibody and the test protein are then mixed and contacted with Factor XII or a region thereof or a cell expressing same. The level of labelled 3F7 or 3F7G is then determined and compared to the level determined when the labelled antibody is contacted with the Factor XII, region or cells in the absence of the protein. If the level of labelled 3F7 or 3F7G is reduced in the presence of the test protein compared to the absence of the protein, the protein is considered to competitively inhibit binding of 3F7 or 3F7G to Factor XII.

Optionally, the test protein is conjugated to different label to 3F7 or 3F7G. This alternate labelling permits detection of the level of binding of the test protein to Factor XII or the region thereof or the cell.

In another example, the protein is permitted to bind to Factor XII or a region thereof or a cell expressing same prior to contacting the Factor XII, region or cell with 3F7 or 3F7G. A reduction in the amount of bound 3F7 or 3F7G in the presence of the protein compared to in the absence of the protein indicates that the protein competitively inhibits 3F7 or 3F7G binding to Factor XII. A reciprocal assay can also be performed using labelled protein and first allowing 3F7 or 3F7G to bind to Factor XII. In this case, a reduced amount of labelled protein bound to Factor XII in the presence of 3F7 or 3F7G compared to in the absence of 3F7 or 3F7G indicates that the protein competitively inhibits binding of 3F7 or 3F7G to Factor XII.

### FXIIa Amidolytic Activity

In one example, proteins of the present disclosure inhibit the amidolytic activity of human Factor XIIa. Methods of determining amidolytic activity of the conjugates of the disclosure will be apparent to the skilled person and/or described herein.

In one example, an in vitro assay is used to determine the level of FXIIa amidolytic activity. For example, the amidolytic activity can be measured by assay of the cleavage of FXII in the presence of conjugate of the disclosure and a buffer. For example, FXII is incubated in the presence of absence of a conjugate of the disclosure or control. Following incubation and addition of a detection substrate, the amidolytic activity is spectrophotometrically determined as a change in optical density (i.e., colour change). Proteins that are found to effectively inhibit amidolytic activity are identified as proteins that inhibit FXII activity.

### Visual Appearance

Pharmaceutical formulations encompassed by the present disclosure are assessed for visual appearance to determine, for example, the colour and clarity.

### Dynamic Light Scattering

In one example, the particle size distribution is assessed using dynamic light scattering (DLS). DLS measures light scattered from particles based on Brownian motion and relies on differences in the index of refraction between the particle and the formulation. For example, the fluctuation of light intensity using a digital correlator is measured. The correlation functions are fitted into an analytical program (e.g., Malvern Zetasizer software) to calculate the particle size distribution. For the determination of Z-average hydrodynamic diameter, a cumulants analysis and the Stokes Einstein equation is performed using e.g., the viscosity of water (0.8872 mPa*s) at 25° C. The polydispersity index can also be obtained from the same cumulants analysis. Modality of fit is evaluated based on plots of size distribution versus intensity: modality can be described as monomodal (i.e., one peak) or multimodal (i.e., two or more peaks).

### Micro-Flow Imaging

In one example, sub-visible particles are assessed using micro-flow imaging (MFI). For example, digital images of particles suspended in a fluid are captured and automatically analysed for particle parameters, such as aspect ratio (AR) and intensity. The size (e.g., in μm) and count (i.e., number of particles per ml) can also be obtained. According to this method the data are morphologically categorised as proteinaceous (i.e., circular) and non-proteinaceous (i.e., non-proteinaceous particles such as air bubbles or silicone oil droplets) and a ratio of the non-proteinaceous particles to proteinaceous particles (i.e., the circular fraction) can be determined. A low circular fraction value indicates that the test article is comprised of mostly non-circular, likely proteinaceous particles.

### Size Exclusion Chromatography

In one example, soluble aggregates are assessed using size exclusion chromatography (SEC or SE-HPLC) which separates lower and higher molecular mass variants of the protein, as well as any impurities. According to this method, the results are described as the summation of aggregation peaks (APs) and summation of degradation peaks (DPs). For example, the identity of a pharmaceutical formulation of the present disclosure is determined by comparing the chromatographic retention time of the major peaks with the retention time of the major peak of a reference standard.

### Differential Scanning Fluorimetry (DSF)

In one example, thermal stability of the pharmaceutical formulation of the present disclosure is assessed using differential scanning fluorimetry (DSF). DSF is a fluorescence-based assay using real-time PCR to monitor thermally induced protein denaturation by measuring changes fluorescence of a dye that binds preferentially to unfolded protein. For example, thermal unfolding and aggregation are monitored by changes in intrinsic protein fluorescence and static light scattering, respectively, as a function of temperature. According to this method, the midpoint of thermal transition (Tₘ) and onset of melting temperature (Tₒₙₛₑₜ) are determined by monitoring intrinsic fluorescence. The onset of aggregation temperature (T_{agg}) are determined by monitoring static light scattering, e.g., at 266 nm and 473 nm. Samples of the pharmaceutical formulation can be assessed across a range of temperatures, (e.g., 20-95° C.) with a temperature increase at the rate of e.g., 0.5° C./min.

### Capillary Gel Electrophoresis

In one example, the pharmaceutical formulation of the present disclosure is assessed for stability and/or total accumulation of impurities using capillary gel electrophoresis (CGE). For example, both reduced-CGE (R-CGE) and non-reduced-CGE (NR-CGE) may be performed. In one example, R-GCE and NR-CGE are carried out using a capillary electrophoresis system (e.g., Beckman P/ACE MDQ or PA800) with a capillary length of e.g., 20.2 cm and 10 cm respectively from inlet to detection window, temperature control from e.g., 20 to 40° C. (+2° C.) and detector at e.g., 488 nm excitation.

### Cation Exchange Chromatography

In one example, the pharmaceutical formulation of the present disclosure is assessed for total charged variants using cation exchange (CEX) chromatography. CEX chromatography separates proteins according to their overall charge under native conditions. The CEX analysis is used to determine the purity of the product by separating the acidic and basic variants. The protein of interest must have a charge opposite to that of the functional group attached to the resin of the column in order to bind. Elution of the protein is achieved by increasing the ionic strength breaking the ionic interaction between the protein and the resin. The chromatographic technique separates the acidic, neutral and basic variants of a sample based on ionic strength. The peaks of interest are observed by UV detection at 280 nm where the acidic variants eluting first followed by neutral and basic variants. In one example CEX chromatography is carried out using a high performance liquid chromatography (HPLC) system (e.g. Dionex UltiMate 3000 BioRS (U) HPLC).

### Gibbs Free Energy (ΔG_{trend}; HUNK)

In one example, the chemical stability and aggregation behaviour of a pharmaceutical formulation of the present disclosure is evaluated by the change in the Gibbs free energy or ΔG_{trend} (HUNK) analysis. The ΔG_{trend} analysis measures the relationship between ΔG of protein unfolding and protein aggregation as a function of protein concentration. In the absence of aggregation, the AG of protein unfolding is a unimolecular process independent of protein concentration. If a change in ΔG is observed as a function of protein concentration, it signifies presence of aggregation. According to this method, there are two possible relationships between ΔG of protein unfolding and protein concentration if aggregation occurs:
1. ΔG_{trend} increases with protein concentration: This relationship indicates the presence of native state aggregation−the ΔG of protein unfolding increases (becomes more positive) as a function of protein concentration (i.e., concentration of native protein aggregates increases as a function of protein concentration); or
2. ΔG_{trend} decreases with protein concentration: This relationship indicates the presence of denatured state aggregation−the AG of protein unfolding decreases (become less positive) as a function of protein concentration (i.e., concentration of denatured protein aggregates increases as a function of protein concentration).

In a HUNK experiment the ΔG of protein unfolding is determined isothermally by measuring changes in a protein's intrinsic fluorescence spectrum (i.e., emission from tryptophan residues) as it unfolds in the presence of increasing amounts of denaturant.

In one example, ΔG_{trend} is determined by measuring AG of the protein unfolding at varying concentrations (e.g., 0.25, 0.6, 2.5, 6.0, 25.0 mg/ml) diluted to target concentration in a buffer of the pharmaceutical formulation of the disclosure. Each concentration level is titrated with increasing denaturant concentration (e.g., 32-point curve spanning urea concentration 2.00-8.74 M) while fluorescence spectra is measured from 300-500 nm (excitation 280 nm) with a slit width of 10 nm. The emission spectrum wavelength ratio of 350 nm/330 nm is plotted against urea concentration for each sample concentration level, and ΔG of protein unfolding determined using a 2 state (i.e., one transition) model fit. Determined ΔG values are plotted against sample concentration to determine ΔG_{trend}.

### Osmolality

In one example, the osmolality of the pharmaceutical formulation of the present disclosure is assessed.

### Turbidity Assessed by Absorbance at 550 nm

In one example, the turbidity of the pharmaceutical formulation of the present disclosure is assessed. For example, the turbidity is assessed using a spectrophotometer and measuring the absorbance at 550 nm.

### Syringeability

In one example, the syringeability of the pharmaceutical formulation of the present disclosure is assessed. For example, the formulation is expelled with a 2 ml syringe, 10 ml syringe, or left untreated as a pre-expulsion control. According to this method, the syringe plunger is pushed through the 2 ml syringes at a linear speed of 0.2 in/min and through the 10 ml syringes at 0.6 in/min until the plunger reaches the bottom and reaches the force of 30 N. Break-loose (BF) and glide (GF) forces are measured during expulsion and used to assess application suitability. Break-loose force describes the force required to initiate movement of the plunger (the initial 0.3 mm for 2 ml syringe and 0.5 mm for 10 ml syringe). Glide force Max refers to the maximum friction force required to sustain plunger movement. The maximum force value is measured from the end of the break loose region to the end of the glide force region (26 mm for 2 ml syringe and 24 mm for 10 ml syringe) prior to the point where the force reaches 30 N).

### Uses of the Pharmaceutical Formulation

As discussed herein, the present disclosure provides a method of treating or preventing a disease or condition in a subject, comprising administering a pharmaceutical formulation of the present disclosure to the subject. In one example, the present disclosure provides a method of treating or preventing a disease or condition in a subject in need thereof.

The present disclosure also provides for use of a pharmaceutical formulation of the present disclosure for treating or preventing a disease or condition in a subject comprising administering the pharmaceutical formulation of the present disclosure to the subject. In one example, the present disclosure provides for use of a pharmaceutical formulation of the present disclosure for treating or preventing a disease or condition in a subject in need thereof.

The present disclosure also provides a method of antagonising activity of Factor XII and/or activated Factor XII in a subject, the method comprising administering the high concentration formulation of the present disclosure to the subject. In one example, the present disclosure provides a method of antagonising activity of Factor XII and/or activated Factor XII in a subject in need thereof.

In one example, the disease or condition is a thrombotic disorder, an inflammatory disorder and/or a thrombo-inflammatory disorder.

In one example, the subject suffers from a thrombotic disorder, an inflammatory disorder and/or a thrombo-inflammatory disorder. The thrombotic disorder, inflammatory disorder and/or thrombo-inflammatory disorder can be inherited or acquired. For example, a subject suffering from a thrombotic disorder, an inflammatory disorder and/or a thrombo-inflammatory disorder has suffered a symptom of a thrombotic disorder, an inflammatory disorder and/or a thrombo-inflammatory disorder.

In one example, the thrombotic disorder, inflammatory disorder and/or thrombo-inflammatory disorder is selected from the group consisting of venous, arterial or capillary thrombus formation (such as stroke, myocardial infarction, deep vein thrombosis (DVT), portal vein thrombosis, renal vein thrombosis, jugular vein thrombosis, cerebral sinus thrombosis, Budd-Chiari syndrome, Paget-Schroetter disease or silent brain ischemia), thrombus formation in the heart, thromboembolism, thrombus formation during and/or after contacting blood of a human or animal subject with artificial surfaces, disseminated intravascular coagulation (DIC), atrial fibrillation, acute coronary syndromes (ACS), atherosclerotic disease, ischaemic stroke with reperfusion, a disease associated with ischemia-reperfusion injury (IRI, such as trauma, organ transplantation), neurotraumatic disorder (such as such as traumatic brain injury, spinal cord injury), a neurological inflammatory disease (such as multiples sclerosis), an interstitial lung disease (such as idiopathic pulmonary fibrosis (IPF)), pneumonia, fibrinolysis, a disease related to FXII/FXIIa-induced kinin formation (such as hereditary angioedema (HAE)), sepsis, a disease related to FXII/FXIIa-mediated complement activation, acute respiratory distress syndrome (ARDS), organ and cell transplantation, sickle cell disease and a condition associated with increased vascular permeability.

In one example, the pharmaceutical formulation of the present disclosure is administered to the subject in an amount to reduce the severity of the disease or condition in the subject.

In one example, the subject is at risk of developing a thrombotic disorder, an inflammatory disorder and/or a thrombo-inflammatory disorder. A subject is at risk if he or she has a higher risk of developing a thrombotic disorder, an inflammatory disorder and/or a thrombo-inflammatory disorder than a control population. The control population may include one or more subjects selected at random from the general population (e.g., matched by age, gender, race and/or ethnicity) who have not suffered from or have a family history of a thrombotic disorder, an inflammatory disorder and/or a thrombo-inflammatory disorder. A subject can be considered at risk for a disease or condition if a “risk factor” associated with a thrombotic disorder, an inflammatory disorder and/or a thrombo-inflammatory disorder is found to be associated with that subject. A risk factor can include any activity, trait, event or property associated with a given disorder, for example, through statistical or epidemiological studies on a population of subjects. A subject can thus be classified as being at risk for a thrombotic disorder, an inflammatory disorder and/or a thrombo-inflammatory disorder even if studies identifying the underlying risk factors did not include the subject specifically.

In one example, the subject is at risk of developing a thrombotic disorder, an inflammatory disorder and/or a thrombo-inflammatory disorder and the pharmaceutical formulation of the present disclosure is administered before or after the onset of symptoms of a thrombotic disorder, an inflammatory disorder and/or a thrombo-inflammatory disorder. In one example, the pharmaceutical formulation is administered before the onset of symptoms of a thrombotic disorder, an inflammatory disorder and/or a thrombo-inflammatory disorder. In one example, the pharmaceutical formulation is administered after the onset of symptoms of a thrombotic disorder, an inflammatory disorder and/or a thrombo-inflammatory disorder. In one example, the pharmaceutical formulation of the present disclosure is administered at a dose that alleviates or reduces one or more of the symptoms of a thrombotic disorder, an inflammatory disorder and/or thrombo-inflammatory disorder in a subject at risk.

The methods of the present disclosure can be readily applied to any form of a thrombotic disorder, an inflammatory disorder and/or a thrombo-inflammatory disorder in a subject.

In one example, a method of the disclosure reduces any symptom of a thrombotic disorder, an inflammatory disorder and/or a thrombo-inflammatory disorder known in the art and/or described herein.

As will be apparent to the skilled person a “reduction” in a symptom of a disorder in a subject will be comparative to another subject who also suffers from a disorder but who has not received treatment with a method described herein. This does not necessarily require a side-by-side comparison of two subjects. Rather population data can be relied upon. For example, a population of subjects suffering from a thrombotic disorder, an inflammatory disorder and/or a thrombo-inflammatory disorder who have not received treatment with a method described herein (optionally, a population of similar subjects to the treated subject, e.g., age, weight, race) are assessed and the mean values are compared to results of a subject or population of subjects treated with a method described herein.

A method of the present disclosure may also include co-administration of the pharmaceutical formulation according to the disclosure together with the administration of another therapeutically effective agent for the prevention or treatment of a thrombotic disorder, an inflammatory disorder and/or a thrombo-inflammatory disorder.

In one example, the pharmaceutical formulation of the disclosure is used in combination with at least one additional known compound or therapeutic protein which is currently being used or is in development for preventing or treating thrombotic disorders, inflammatory disorders and/or thrombo-inflammatory disorders, or antagonising activity of Factor XII and/or an activated form thereof. Compounds currently used in the treatment of thrombotic disorders, inflammatory disorders and/or thrombo-inflammatory disorders are known in the art, and include for example, vitamin K antagonists (e.g., warfarin), heparin (e.g., unfractionated or low-molecular weight heparin), synthetic pentasaccharides (e.g., fondaparinux and idraparinux), direct inhibitors of Factor Xa and thrombin (e.g., rivaroxaban, lepirudin, desirudin and dabigatran), cyclooxygenase inhibitors (e.g., aspirin, rofecoxib and valdecoxib), ADP-receptor antagonists (clopidogrel, prasugrel), protease-activated-receptor-1 inhibitors (i.e., PAR1), α_{IIb}β₃-integrin inhibitors (e.g., abciximab and eptifibatide) and statins (e.g., Lovastatin, Pravastatin, Rosuvastatin, Simvastatin, Atorvastatin, and Fluvastatin).

As will be apparent from the foregoing, the present disclosure provides methods of concomitant therapeutic treatment of a subject, comprising administering to a subject in need thereof an effective amount of a first agent and a second agent, wherein the first agent is a pharmaceutical formulation of the present disclosure, and the second agent is also for the prevention or treatment of a thrombotic disorder, an inflammatory disorder and/or a thrombo-inflammatory disorder.

As used herein, the term “concomitant” as in the phrase “concomitant therapeutic treatment” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agent, wherein the second or additional agent, for example, may have been previously administered. A concomitant therapeutic treatment may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g. a human).

### Kits and Other Compositions of Matter

Another example of the disclosure provides kits containing a pharmaceutical formulation of the present disclosure useful for the treatment or prevention of a disease or condition as described above.

In one example, the kit comprises (a) a container comprising a pharmaceutical formulation of the present disclosure; and (b) a package insert with instructions for treating or preventing a disease or condition in a subject.

In one example, the kit comprises (a) at least one pharmaceutical formulation of the present disclosure; (b) instructions for using the kit in treating or preventing the disease or condition in the subject; and (c) optionally, at least one further therapeutically active compound or drug.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating or preventing a blood coagulation disorder and may have a sterile access port (for example, the container may be an intravenous solution bag or a vial having a stopper pierceable by a hypodermic injection needle). The label or package insert indicates that the composition is used for treating a subject eligible for treatment, e.g., one having or predisposed to developing a thrombotic disorder, an inflammatory disorder and/or a thrombo-inflammatory disorder, with specific guidance regarding dosing amounts and intervals of the pharmaceutical formulation and any other medicament being provided. The kit may further include other materials desirable from a commercial and user standpoint, including filters, needles, and syringes. In some examples of the present disclosure, the formulation can be present in an injectable device (e.g., an injectable syringe, e.g., a prefilled injectable syringe). The syringe may be adapted for individual administration, e.g., as a single vial system including an autoinjector (e.g., a pen-injector device). In one example, the injectable device is a prefilled pen or other suitable autoinjectable device, optionally with instruction for use and administration.

The kit optionally further comprises a container comprising a second medicament, wherein the pharmaceutical formulation is a first medicament, and which article further comprises instructions on the package insert for treating the subject with the second medicament, in an effective amount. The second medicament may be a therapeutic protein set forth above.

In one example, the disclosure provides a prefilled syringe or autoinjector comprising a formulation of the present disclosure. In one example, the prefilled syringe is a glass luer syringe with plunger.

In one example, the disclosure provides a vial comprising a formulation of the disclosure.

The present disclosure includes the following non-limiting Examples.

### EXAMPLES

### Example 1: Physical and Chemical Stability of Excipients of the Formulation

To assess formulation excipients a biophysical and excipient screening study was performed, as well as a solubility study. The pre-formulation experiments were conducted using a monoclonal anti-Factor XII antibody (affinity matured 3F7 or 3F7^{aff} as described herein).

### Baseline Biophysical Screening Study

The physical and thermal stability of the anti-Factor XII antibody in ten buffer types (F1-F10, Table 1) was evaluated. Using volumetric dilution and 320 nm baseline correction, the concentration target of 100 mg/ml was achieved for each formulation.

The protein concentrations of the formulations were determined by measuring the absorbance (A_{280/320}) and pH was assessed using a calibrated pH Meter.

In the case of buffer exchanges with 20 mM glutamate buffer at pH 5.5 (F1), it was observed that the antibody solution formulated at 100 mg/ml did not reach the pH target (measured pH was 5.65) even after extensive buffer exchanges.

Particle distribution and thermal stability of antibody solutions were investigated by dynamic light scattering (DLS) and differential scanning fluorimetry (DSF).

Dynamic light scattering was performed using at least 100 μl of undiluted sample in an optical-quality plastic cuvette. For each sample measurement, five consecutive scans were acquired at 25° C. (utilizing Malvern's automatic attenuation selection setting) with 3 minutes of equilibration at the beginning of each measurement. The Protein Analysis algorithm (Malvern Zetasizer software) was used as a model for data processing. The Z-average hydrodynamic diameter was calculated from a cumulants analysis and the Stokes Einstein equation, using the viscosity of water (0.8872 mPa*s) at 25° C. The polydispersity index (PDI) was obtained from the same cumulants analysis. Modality of fit was evaluated based on plots of size distribution versus intensity: modality can be described as monomodal (i.e. one peak) or multimodal (i.e. two or more peaks).

In the DLS study, formulations were compared with respect to Z-average diameter and PDI (Table 1). Clear trends related to pH were observed for Z-average in succinate (F2-F4) and citrate buffers (F8-F10), where the Z-average values increased with increasing pH. The trend observed for Z-average values, however, did not correspond with changes in PDI, which suggests that elevated Z-average values are related to stronger, unspecific electrostatic interactions between antibody molecules at higher pH. As opposed to the samples formulated in succinate and citrate buffers, a slight upward trend as a function of pH was observed for PDI in histidine buffer (F5-F7), with no changes in Z-average values, which were lower compared to succinate and citrate formulations. Similar results as for histidine buffer were observed for glutamate buffer (F1) where elevated PDI (compared to succinate and citrate buffers) and low Z-average were measured.

The DSF experiments were performed using an Unchained Labs UNit. Tested formulations were screened for thermal stability and formation of soluble aggregates. The thermal unfolding and aggregation were monitored by changes in intrinsic protein fluorescence and static light scattering, respectively, as a function of temperature. The midpoint of thermal transition (Tₘ) and onset of melting temperature (Tₒₙₛₑₜ) were determined by monitoring intrinsic fluorescence. The onset of aggregation temperature (T_{agg}) was determined by monitoring static light scattering at 266 nm and 473 nm. Each DSF analysis was run in triplicate using 8.8 μl at a protein concentration of 100 mg/ml. Samples were analysed across a range of temperatures (20-95° C.) with a temperature increase at the rate of 0.5° C./min.

In DSF analysis the thermal stability and soluble aggregates formation were evaluated. The Tₘ, Tₒₙₛₑₜ, and T_{agg} values determined for all tested formulations are shown in Table 1.

The Tₒₙₛₑₜ and Tₘ values obtained for glutamate and histidine buffers were slightly higher compared to succinate and citrate and indicate higher thermal stability of formulations F1 and F5-F7. No significant differences in thermal stability were observed for different pH values. The onset aggregation temperatures and static light scattering intensities at 266 nm and 473 nm showed higher T_{agg} values and lower SLS intensities for glutamate (F1) and histidine buffers

TABLE 1
Summary of results in Baseline Biophysical Screening

| | | | DLS Analysis | | | | | |
|---|---|---|---|---|---|---|---|---|
| | | | Mean | | DSF Analysis | | | |
| | | | Z-Average | | Mean | Mean | Mean T_{agg} | Mean T_{agg} |
| | | | Diameter | Mean | Tₘ | Tₒₙₛₑₜ | (° C.) | (° C.) |
| Formulation | Buffer | pH | (nm) | polydispersity | (° C.) | (° C.) | [473 nm] | [266 nm] |
| F1 | 20 mM Glutamate | 5.5 | 19.05 | 0.078 | 65.7 | 60.3 | 61.5 | 62.5 |
| F2 | 20 mM Succinate | 5.5 | 10.28 | 0.129 | 62.2 | 59.7 | 60.8 | 59.8 |
| F3 | 20 mM Succinate | 6.0 | 24.04 | 0.082 | 61.1 | 59.0 | 59.9 | 59.0 |
| F4 | 20 mM Succinate | 6.5 | 34.64 | 0.070 | 61.3 | 585 | 59.1 | 58.8 |
| F5 | 20 mM Histidine | 5.5 | 48.09 | 0.091 | 64.5 | 58.6 | 60.6 | 61.4 |
| F6 | 20 mM Histidine | 6.0 | 11.21 | 0.116 | 65.0 | 60.1 | 62.0 | 60.4 |
| F7 | 20 mM Histidine | 6.5 | 13.68 | 0.149 | 5.4 | 60.2 | 61.1 | 61.3 |
| F8 | 20 mM Citrate | 5.5 | 12.69 | 0.216 | 59.6 | 57.5 | 58.0 | 56.6 |
| F9 | 20 mM Citrate | 6.0 | 37.87 | 0.057 | 60.1 | 57.6 | 58.3 | 58.8 |
| F10 | 20 mM Citrate | 6.5 | 40.72 | 0.068 | 60.4 | 57.8 | 58.5 | 57.7 |

(F5-F7), compared to succinate (F2-F4) and citrate (F8-F10) and indicated higher thermal aggregation stability of the antibody. No significant differences in thermal aggregation stability were observed for different pH values in a given buffer.

Based on the DLS and DSF results the formulations containing glutamate and histidine at pH 5.5 exhibited the most positive effect on thermal and aggregation stability of the anti-FXII antibody.

### Excipient Screen

Based on Baseline Biophysical screen, twelve formulations were evaluated to assess the effect of four different excipients (phenylalanine, proline, arginine, and sorbitol) in three different buffers (glutamate, succinate, and histidine) at pH 5.5 on the stability of the antibody at concentration of 100 mg/ml (F11-F22, Table 2). Additionally, a formulation containing glutamate and histidine at pH 5.5 was evaluated (F23). The effect of five different excipients on antibody stability was analysed by DLS and DSF, using methods described above.

In the DLS study, there were no significant differences observed in Z-average and PDI between formulations with and without (±) excipient (Table 2). Similarly, no appreciable differences were observed between tested buffers. The most noticeable effect on Z-average and PDI was observed for arginine in succinate buffer (F17), where both parameters were reduced, compared to other excipients. Overall, no significant differences in distribution of particle sizes were observed between tested formulations.

The Tₒₙₛₑₜ and Tₘ determined for the tested formulations by DSF analysis are shown in Table 2. The comparison of Tₒₙₛₑₜ and Tₘ values showed comparable thermal stability for all formulations containing phenylalanine, proline and sorbitol. A similar stability was observed for formulation F23, containing histidine and glutamate. The lowest thermal stability was observed for formulations containing arginine. The comparison of obtained results showed an elevated thermal aggregation of antibody in all formulations containing succinate and/or arginine compared to other tested formulations. A similar result was obtained for formulation F23, containing histidine and glutamate.

Based on the DLS and DSF results the succinate buffer was excluded from further investigation due to increased propensity towards aggregation of the unfolded protein at elevated temperature. The presence of sorbitol and phenylalanine in tested formulations did not exhibit a significant effect on antibody stability compared to proline and, therefore, both excipients were excluded from further investigation.

TABLE 2
Summary of Results in Excipient Screen

| | | | | DLS Analysis | | | | | |
|---|---|---|---|---|---|---|---|---|---|
| | | | | Mean Z- | | DSF Analysis | | | |
| | | | | Average | | Mean | Mean | Mean T_{agg} | Mean T_{agg} |
| | | | | Diameter | Mean | Tₘ | Tₒₙₛₑₜ | (° C.) | (° C.) |
| Formulation | Buffer | Excipient | pH | (nm) | polydispersity | (° C.) | (° C.) | [473 nm] | [266 nm] |
| F11 | 20 mM Glutamate | 50 mM Phenylalanine | 5.5 | 11.72 | 0.157 | 64.9 | 60.5 | 61.7 | 63.2 |
| F12 | 20 mM Glutamate | 150 mM Proline | 5.5 | 10.57 | 0.143 | 65.9 | 61.0 | 62.4 | 64.6 |
| F13 | 20 mM Glutamate | 150 mM Arginine | 5.5 | 16.42 | 0.090 | 61.3 | 58.9 | 60.0 | 59.7 |
| F14 | 20 mM Glutamate | 250 mM Sorbitol | 5.5 | 12.88 | 0.192 | 66.6 | 62.2 | 63.3 | 64.2 |
| F15 | 20 mM Succinate | 50 mM Phenylalanine | 5.5 | 21.72 | 0.081 | 61.6 | 59.2 | 60.2 | 58.9 |
| F16 | 20 mM Succinate | 150 mM Proline | 5.5 | 21.38 | 0.072 | 62.7 | 60.2 | 61.2 | 60.6 |
| F17 | 20 mM Succinate | 150 mM Arginine | 5.5 | 16.04 | 0.051 | 60.8 | 58.6 | 59.8 | 59.1 |
| F18 | 20 mM Succinate | 250 mM Sorbitol | 5.5 | 24.95 | 0.084 | 63.2 | 61.0 | 62.0 | 61.2 |
| F19 | 20 mM Histidine | 50 mM Phenylalanine | 5.5 | 11.35 | 0.114 | 63.8 | 58.7 | 60.4 | 62.3 |
| F20 | 20 mM Histidine | 150 mM Proline | 5.5 | 10.97 | 0.134 | 64.6 | 59.4 | 60.0 | 62.0 |
| F21 | 20 mM Histidine | 150 mM Arginine | 5.5 | 15.65 | 0.045 | 60.8 | 58.0 | 59.4 | 58.6 |
| F22 | 20 mM Histidine | 250 mM Sorbitol | 5.5 | 12.76 | 0.202 | 65.2 | 60.2 | 61.6 | 63.1 |
| F23 | 20 mM Histidine | | 5.5 | 18.16 | 0.061 | 63.4 | 61.0 | 61.9 | 61.2 |
| | 150 mM Glutamate | | | | | | | | |

### Solubility Study

Based on the results of the Excipient Screening study six formulations were evaluated to assess the solubility of the antibody at a concentration of ≥200 mg/ml. Two of the tested formulations contained 150 mM glutamate buffer at pH 5.5, with 20 mM histidine (F23) and without 20 mM histidine (F24). The four other formulations contained glutamate or histidine buffer at pH 5.5 with proline or arginine as excipients (F12, F13, F20, F21; Table 3). Two sets of samples were prepared and the appearance evaluated. The first set of six control samples was concentrated to 100 mg/ml. The second set of six solubility samples was concentrated to antibody concentration of ≥200 mg/ml.

Visual appearance (i.e., colour, clarity, and particulates) was assessed according to GTM-0033-03, “Appearance by Visual Evaluation.” The type of the observed particles was judged to be product or non-product related. The control solutions (i.e., 100 mg/ml) were slightly yellow; whereas, the concentrated solubility solutions (i.e., 200 mg/ml), due to high protein concentration, were slightly brown. The control and concentrated solubility solutions were slightly hazy and hazy liquids, respectively, and free of product related visible particles. After performing appearance testing the concentrated solubility solutions (200 mg/ml) were diluted in corresponding buffers to 100 mg/ml (solubility solutions) and tested with control samples by DLS, as described above, and SEC methods described below.

The DLS analysis was performed on each set of controls and solubility solutions and the results are shown in Table 3. The results obtained for all control and solubility solutions showed comparable Z-average and PDI (≤0.13) values.

Size exclusion chromatography (SEC) was performed by separating the high molecular weight species, main monomer and fragments under native conditions, using the Dionex UltiMate 3000 BioRS HPLC system. As shown in Table 3, there was a slight increase of high molecular weight species in solubility solutions, by 0.4-1.0%, compared to controls. No significant changes in percentage fragments were observed between solubility and control samples.

In summary, the concentrated samples (≥200 mg/ml) remained soluble and no negative effects on antibody stability were observed for all tested formulations.

TABLE 3
Summary of Results in Solubility Screen

| | | | | DLS Analysis | | | | |
|---|---|---|---|---|---|---|---|---|
| | | | | Mean Z- | | SEC Analysis | | |
| | | | | Average | | | % Main | |
| | | | | Diameter | Mean | % HMW | monomer | % LMW |
| Formulation | Buffer | Excipient | pH | (nm) | polydispersity | species | peak | species |
| F12 (control) | 20 mM Glutamate | 150 mM Proline | 5.5 | 11.90 | 0.100 | 2.0 | 97.2 | 0.8 |
| F12 (solub.) | 20 mM Glutamate | 150 mM Proline | 5.5 | 12.34 | 0.133 | 3.0 | 96.2 | 0.8 |
| F13 (control) | 20 mM Glutamate | 150 mM Arginine | 5.5 | 15.51 | 0.030 | 1.4 | 97.8 | 0.8 |
| F13 (solub.) | 20 mM Glutamate | 150 mM Arginine | 5.5 | 16.29 | 0.046 | 2.1 | 97.1 | 0.8 |
| F20 (control) | 20 mM Histidine | 150 mM Proline | 5.5 | 11.88 | 0.115 | 1.6 | 97.6 | 0.8 |
| F20 (solub.) | 20 mM Histidine | 150 mM Proline | 5.5 | 10.80 | 0.128 | 2.3 | 96.9 | 0.8 |
| F21 (control) | 20 mM Histidine | 150 mM Arginine | 5.5 | 15.96 | 0.046 | 1.3 | 98.0 | 0.8 |
| F21 (solub.) | 20 mM Histidine | 150 mM Arginine | 5.5 | 15.88 | 0.042 | 1.7 | 97.5 | 0.8 |
| F23 (control) | 20 mM Histidine | | 5.5 | 19.05 | 0.069 | 1.4 | 97.8 | 0.7 |
| | 150 mM Glutamate | | | | | | | |
| F23 (solub.) | 20 mM Histidine | | 5.5 | 18.79 | 0.058 | 1.9 | 97.4 | 0.8 |
| | 150 mM Glutamate | | | | | | | |
| F24 (control) | 150 mM Glutamate | | 5.5 | 18.85 | 0.056 | 1.6 | 97.6 | 0.8 |
| F24 (solub.) | 150 mM Glutamate | | 5.5 | 17.92 | 0.044 | 2.1 | 97.2 | 0.8 |

### Surfactant Screening

The protective effect of non-ionic surfactants on antibody soluble and insoluble aggregates formation was evaluated for polysorbate 80 (PS-80), polysorbate 20 (PS-20), and Poloxamer 188 in freeze-thaw and agitation studies. A total of fourteen formulations with antibody concentrations of 100 mg/ml were screened in freeze-thaw and agitation studies (F21 and F24 to F36; Table 4). For the freeze-thaw studies all formulations were subjected to 3 freeze-thaw cycles at −75±10° C. Samples were stored at −75±10° C. for a minimum of 1 hour and then thawed at room temperature for 1 hour. Respective control samples were prepared by incubating at 5±3° C. For the agitation studies formulations were agitated on a lab rotator (˜50 rpm) for approximately 50 hours at room temperature. Respective control samples were prepared by incubating at 5±3° C. All samples were analysed by appearance, size exclusion chromatography and differential light scattering as described above.

In both the freeze-thaw and agitation studies, all stressed samples remained unchanged in terms of colour (i.e., slightly yellow), clarity (i.e., clear), and particle content (i.e., free of visible particles) in comparison to the control samples.

The DLS results showed an increase in the PDI was seen in histidine and glutamate formulations without surfactant that were subjected to F/T and agitation stresses, compared to control samples. The highest increase in PDI (by 0.09) was observed for F21 agitation sample, compared to control, which may suggest the presence of particles in the stressed sample. Similar differences were not observed for any of freeze-thaw and agitated samples containing surfactant. The Z-average and PDI values measured for stressed samples were comparable to controls.

No significant changes in aggregates concentration were detected by SEC for freeze-thaw and agitated samples, compared to the corresponding controls. Percent area of the monomer peak ranged from 97.5% to 98.4%, percent area of high molecular weight species ranged from 1.0% to 1.9%, and percent area of low molecular weight species ranged from 0.6% to 0.8%.

Sub-visible particles were measured by Micro-Flow Imaging (MFI) using an MFI 5200 with Automated Pipet System to characterize the size, concentration, and morphology of particles present in the samples. The analyses were performed with single measurements using 700 or 1000 μl of neat sample and cumulative counts per ml for ≥2 μm, ≥5 μm, ÷10 μm and ÷25 μm sized particles determined (for 1-100 μm sized particles). Additionally, ≥5 μm sized particles with an aspect ratio (AR) of ≥0.85 were processed using morphological categorisation parameters within the MFI software (MVAS) to determine the circular fraction. A low circular fraction value indicates that the test article is comprised of mostly non-circular, likely proteinaceous particles.

The absence of surfactant in control samples (F21 and F24) subjected to freeze-thaw and agitation stresses appeared to have a significant impact on sub-visible particles formation. The increases in particle counts were observed for both formulations lacking surfactant. The only exception was formulation F24 subjected to agitation stress, which exhibited particle counts comparable to control sample. In contrast to results obtained for control samples, any type of surfactant present in samples subjected to freeze-thaw and agitation stress significantly suppressed formation of sub-visible particles. The suppression effect was similar for all tested surfactants; however, the reduction of particles observed for formulations containing PS-80 was the most beneficial. Also, the protective effect observed was slightly better for PS-80 concentration of 0.05% than for 0.02%.

TABLE 4
Formulations used in freeze-thaw and agitation studies

| Formulation | Buffer | | Excipient | Surfactant | pH |
|---|---|---|---|---|---|
| F21 | 20 | mM Histidine | 150 mM Arginine | | 5.5 |
| F24 | 150 | mM Glutamate | | | 5.5 |
| F25 | 20 | mM Histidine | 150 mM Arginine | 0.05% PS-80 | 5.5 |
| F26 | 150 | mM Glutamate | | 0.05% PS-80 | 5.5 |
| F27 | 20 | mM Histidine | 150 mM Arginine | 0.02% PS-80 | 5.5 |
| F28 | 150 | mM Glutamate | | 0.02% PS-80 | 5.5 |
| F29 | 20 | mM Histidine | 150 mM Arginine | 0.05% PS-20 | 5.5 |
| F30 | 150 | mM Glutamate | | 0.05% PS-20 | 5.5 |
| F31 | 20 | mM Histidine | 150 mM Arginine | 0.02% PS-20 | 5.5 |
| F32 | 150 | mM Glutamate | | 0.02% PS-20 | 5.5 |
| F33 | 20 | mM Histidine | 150 mM Arginine | 0.05% Poloxamer 188 | 5.5 |
| F34 | 150 | mM Glutamate | | 0.05% Poloxamer 188 | 5.5 |
| F35 | 20 | mM Histidine | 150 mM Arginine | 0.02% Poloxamer 188 | 5.5 |
| F36 | 150 | mM Glutamate | | 0.02% Poloxamer 188 | 5.5 |

### Example 2: Design of Experiment Study

The results from the baseline biophysical screening, excipient screening, solubility, and surfactant screening studies were used to design formulation buffers for this study. A total of 30 formulations were tested in the accelerated stability of the antibody (Table 5). Formulation F_{DOE}12 was chosen as a centre point (100 mg/ml antibody, 20 mM Glutamate, 100 mM Arginine, 0.05% PS-80, pH 5.5). In addition, the original formulation (F_{original}) was used as a control. The samples were stored at 5° C. and 40° C. and analysed at time-zero, at two weeks and at four weeks.

TABLE 5
Formulations used in Design of Experiment Studies

| | Ab conc. | PS-80 | Glutamate | Histidine | Proline | Arginine | |
|---|---|---|---|---|---|---|---|
| Form. | (mg/ml) | (%) | (mM) | (mM) | (mM) | (mM) | pH |
| F_{DOE}1 | 100 | 0.05 | 20 | | | | 5.0 |
| F_{DOE}2 | 100 | 0.05 | 20 | | | | 5.3 |
| F_{DOE}3 | 100 | 0.05 | 20 | | | | 5.5 |
| F_{DOE}4 | 100 | 0.05 | 20 | | 100 | | 5.0 |
| F_{DOE}5 | 100 | 0.05 | 20 | | 100 | | 5.3 |
| F_{DOE}6 | 100 | 0.05 | 20 | | 100 | | 5.5 |
| F_{DOE}7 | 100 | 0.05 | 20 | | 200 | | 5.0 |
| F_{DOE}8 | 100 | 0.05 | 20 | | 200 | | 5.3 |
| F_{DOE}9 | 100 | 0.05 | 20 | | 200 | | 5.5 |
| F_{DOE}10 | 100 | 0.05 | 20 | | | 100 | 5.0 |
| F_{DOE}11 | 100 | 0.05 | 20 | | | 100 | 5.3 |
| F_{DOE}12 | 100 | 0.05 | 20 | | | 100 | 5.5 |
| F_{DOE}13 | 100 | 0.05 | 20 | | | 200 | 5.0 |
| F_{DOE}14 | 100 | 0.05 | 20 | | | 200 | 5.3 |
| F_{DOE}15 | 100 | 0.05 | 20 | | | 200 | 5.5 |
| F_{DOE}16 | 100 | 0.05 | | 20 | | | 5.5. |
| F_{DOE}17 | 100 | 0.05 | | 20 | | | 5.7 |
| F_{DOE}18 | 100 | 0.05 | | 20 | | | 6.0 |
| F_{DOE}19 | 100 | 0.05 | | 20 | 100 | | 5.5. |
| F_{DOE}20 | 100 | 0.05 | | 20 | 100 | | 5.7 |
| F_{DOE}21 | 100 | 0.05 | | 20 | 100 | | 6.0 |
| F_{DOE}22 | 100 | 0.05 | | 20 | 200 | | 5.5. |
| F_{DOE}23 | 100 | 0.05 | | 20 | 200 | | 5.7 |
| F_{DOE}24 | 100 | 0.05 | | 20 | 200 | | 6.0 |
| F_{DOE}25 | 100 | 0.05 | | 20 | | 100 | 5.5. |
| F_{DOE}26 | 100 | 0.05 | | 20 | | 100 | 5.7 |
| F_{DOE}27 | 100 | 0.05 | | 20 | | 100 | 6.0 |
| F_{DOE}28 | 100 | 0.05 | | 20 | | 200 | 5.5. |
| F_{DOE}29 | 100 | 0.05 | | 20 | | 200 | 5.7 |
| F_{DOE}30 | 100 | 0.05 | | 20 | | 200 | 6.0 |
| F_{original} | 100 | 0.02 | | 20 | 140 | 150 | 6.1 |

The following analyses were performed on selected samples:
Time-zero: Osmolality, pH, UV, appearance and ΔG_{trend} HUNK analysis
At two weeks: size exclusion chromatography (SEC), capillary gel electrophoresis (reduced (R-CGE) and non-reduced (NR-CGE))
At four weeks: dynamic light scattering (DLS), micro-flow imaging (MFI), SEC, cation exchange chromatography (CEX), pH, R-CGE, NR-CGE and appearance

The osmolality of selected formulations (F_{DOE}9, F_{DOE}15, F_{DOE}19, and F_{DOE}25) was determined prior to storage and the osmolality of the selected formulations was 296 mOsm/kg (F_{DOE}9), 435 mOsm/kg (F_{DOE}15), 154 mOsm/kg (F_{DOE}19) and 240 mOsm/kg (F_{DOE}25).

Chemical stability and aggregation behaviour of the antibody in different formulations (F_{DOE}3, F_{DOE}6, F_{DOE}9, F_{DOE}12 and F_{DOE}15) was also assessed prior to storage using ΔG_{trend} (HUNK) analysis.

ΔG_{trend} was determined by measuring AG of unfolding at concentrations of 0.25, 0.6, 2.5, 6.0, 25.0 mg/ml. All samples were diluted to target concentration in formulation buffer. Each concentration level was titrated with increasing denaturant concentration (32-point curve spanning urea concentration 2.00-8.74 M) while fluorescence spectra was measured from 300-500 nm (excitation 280 nm) with a slit width of 10 nm. Gain was adjusted depending on the sample concentration to provide maximum signal without saturating the detector (100 for 0.25 and 0.6 mg/ml; 10 for 2.5 and 6.0 mg/ml; 1 for 25.0 mg/ml). The emission spectrum wavelength ratio of 350 nm/330 nm was plotted against urea concentration for each sample concentration level, and ΔG of protein unfolding was determined using a 2 state (i.e. one transition) model fit. Determined ΔG values were plotted against sample concentration to determine ΔG_{trend}, with a positive ΔG_{trend} (i.e. increasing AG with sample concentration) indicative of self-association of the native state and a negative ΔG_{trend} (i.e. decreasing AG with sample concentration) indicative of aggregation from the denatured state.

ΔG_{trend} analysis performed for F_{DOE}3 showed a gradual increase in ΔG with antibody concentration, and suggests the formation of native protein aggregates in the tested sample. The observed effect was significantly reduced after adding proline to F_{DOE}3 (F_{DOE}6 and F_{DOE}9). The ΔG_{trend} observed for formulations F_{DOE}6 (with 100 mM proline) and F_{DOE}9 (with 200 mM proline) was not dependent on antibody concentration and showed lack of aggregates formation. Interestingly, adding arginine to F_{DOE}3 (F_{DOE}12 and F_{DOE}15) showed a gradual decrease in ΔG with antibody concentration. The decrease effect was dramatic for F_{DOE}15 (with 200 mM arginine) and is evidence for formation of denatured protein aggregates in the tested sample. This result is in good agreement with the DSF data obtained in the excipient screening study where the thermal aggregation of antibody was observed for all formulations containing arginine.

The pH of samples was measured for the initial (i.e., pre-storage) and four week time points. For samples stored for four weeks at 5±3° C. and 40° C./75% relative humidity (RH) the measured pH was within 0.11 pH units of the initial value, for the respective formulation.

The appearance of the initial and four weeks time point samples was evaluated for colour, clarity, and particle content as described above. Samples held for four weeks at 5±3° C. and 40° C./75% RH showed no difference in comparison to the samples at the initial time point. Each sample appeared as a slightly yellow, clear liquid, and free of visible protein related particles.

MFI was performed as described above and the results obtained for samples stored at 5±3° C. and 40° C./75% RH did not reveal any clear trends in cumulative particle counts with respect to storage temperature, buffer or excipient type. A noticeable trend in accumulation of particles as a function of pH was observed for all tested formulations. In general, the smallest difference in the number of particles for samples stored at 5±3° C. and 40° C./75% RH, was observed at pH 5.5 for glutamate buffers and pH 5.5 and pH 5.7 for histidine buffers, for all particle sizes.

The results obtained for R-CGE, NR-CGE, SEC, DLS, and CEX were statistically analysed by Stat-Ease, Inc. Design-Expert® Version 7.0.3. Significance of the responses was evaluated using ANOVA technique at the 95% confidence interval (p values lower than 0.05 indicated a statistically significant correlation). The responses that met this significance threshold were analysed by the software in linear design mode. The calculations were carried out using three factors: pH, proline, and arginine concentration and four responses: accumulation of total impurities (R-CGE), accumulation of total impurities (SEC), PDI difference (DLS), and accumulation of total charged variants (CEX). The p-values obtained by ANOVA for total impurities percent determined by NR-CGE were statistically insignificant (p value=0.34 for glutamate and 0.47 for histidine buffers) and, therefore, the data were excluded from further analysis.

The models generated for the accumulation of total impurities (difference between total impurities obtained for samples stored at 5±3° C. and 40° C./75% RH), detected in R-CGE and SEC analysis for glutamate buffers±proline showed a progressive trend in reduction of impurities as a function of pH. No differences in the accumulation of impurities were observed between glutamate buffers±proline at all tested pH levels.

In R-CGE analysis, the models generated for histidine buffer±proline showed a shallow trend in reduction of impurities as a function of pH, whilst in SEC analysis, the models generated for histidine buffer±proline showed no trend in reduction of impurities as a function of pH. In contrast to glutamate, the presence of proline in histidine buffer slightly reduced the accumulation of impurities, observed mainly at pH 5.5 and pH 5.7. At pH 5.5 the accumulation of impurities was comparable between the glutamate and histidine buffers with and without proline, respectively.

In R-CGE and SEC analysis, the models generated for the accumulation of total impurities in glutamate buffer±arginine showed a progressive trend in reduction of impurities as a function of pH similar to the effects observed for glutamate buffer±proline. In comparison to proline, the presence of arginine in glutamate buffer increased the accumulation of total impurities at all tested PH levels.

In R-CGE analysis, a shallow pH dependent trend in reduction of impurities was observed for histidine buffer±arginine. No significant differences in the accumulation of impurities were observed between histidine buffers±arginine, at all tested PH levels. At pH 5.5 the accumulation of impurities was comparable between the glutamate and histidine buffers with and without arginine, respectively. In contrast, SEC analysis showed no pH dependent trend in accumulation of impurities was observed in models generated for histidine buffer±arginine. In contrast to proline, the presence of arginine in histidine buffer increased the accumulation of total impurities at all tested pH levels.

At pH 5.5 the accumulation of impurities was comparable between the glutamate and histidine buffers with and without arginine, respectively.

The models generated for PDI (difference between PDI obtained for samples stored at 5±3° C. and 40° C./75% RH) determined in DLS analysis for glutamate buffer±proline showed a shallow, decreasing trend in of PDI values as a function of pH. No significant differences in the PDI values were observed between glutamate buffers±proline at all tested pH levels. Similarly, the models generated for PDI in glutamate buffer±arginine showed a shallow, decreasing trend in PDI values as a function of pH. In contrast to proline, the presence of arginine in glutamate buffer significantly increased the PDI values, at all tested pH levels.

The models generated for histidine buffer±proline showed a shallow, increasing trend in of PDI values as a function of pH. No significant differences in the PDI values were observed between histidine buffers±proline, at all tested pH levels. Similarly, the models generated for PDI in histidine buffer±arginine showed a shallow, increasing trend in of PDI values as a function of pH. In contrast to proline, the presence of arginine in histidine buffer significantly increased the PDI values at all tested PH levels.

At pH 5.5 the PDI values were comparable between the glutamate and histidine buffers with and without proline, respectively, as well as with and without arginine.

The models generated for the accumulation of total charged variants (difference between total charged variants obtained for samples stored at 5±3° C. and 40° C./75% RH), detected in CEX analysis for glutamate buffer±proline showed a progressive trend in reduction of charge variants as a function of pH. A slight beneficial effect of proline on reduction of charged variants was observed at all tested pH levels. Similarly, the models generated for the accumulation of charged variants in glutamate buffer±arginine showed a progressive trend in reduction of impurities as a function of pH.

The models generated for histidine buffer±proline showed an upward trend in the accumulation of charged variants as a function of pH. Similar to glutamate buffer, the presence of proline in histidine buffer reduced the accumulation of charged variants at all tested PH levels. Similar to proline, but, more significantly, the presence of arginine decreased the accumulation of total charged variants at all tested PH levels. The models generated for histidine buffer±arginine showed an upward trend in accumulation of charged variants as a function of pH. No differences in the accumulation of impurities were observed between histidine buffers±arginine, at all tested PH levels.

The lowest accumulation of charge variants was observed at pH 5.5 for all tested formulations. No significant differences were observed at pH 5.5 between the proline containing and no proline-containing glutamate and histidine buffers, respectively as well as at pH 5.5 between the glutamate and histidine buffers with and without arginine, respectively. The only exception was glutamate buffer without arginine for which the concentration of charged species was noticeably higher compared to histidine buffer with arginine.

The results from this study showed the highest beneficial effect of proline on antibody stability in glutamate buffer at pH 5.5 and in histidine buffer at pH 5.5-5.7. The protective effect of arginine was observed only for CEX analysis. The DLS, SEC, and R-CGE results showed that the presence of arginine in glutamate and histidine buffers (at tested pH ranges) may decrease the stability of the protein.

### Example 3: Determining Syringeability of a High Concentration Formulation

The suitability of formulations F37 and F38 of the antibody for manufacture and clinical application in a liquid formulation at 100 mg/ml was assessed. Placebo solutions (formulations F37 and F38 without active pharmaceutical ingredient) were tested concurrently as part of the pumping, filter compatibility, and syringeability studies.

Formulation 37: 100 mg/ml antibody; 100 mM glutamate at pH 5.6; 150 mM proline and 0.05% PS-80.

Formulation 38: 100 mg/ml antibody; 20 mM histidine at pH 5.8; 150 mM proline, 80 mM sorbitol and 0.05% PS-80.

### Pumping Study

The pumping study was performed to evaluate if changes in aggregation or degradation could occur during the physical process of pumping antibody-containing material as it would be treated during certain manufacturing processes. To evaluate the stability of each formulation, an initial time point (T0) was collected and compared to samples taken after 60 and 120 minutes of pumping in a series of analytical tests.

### Appearance

The appearance of both placebo and antibody containing samples was assessed by colour, clarity, and relative number of visible particles. All antibody-containing samples evaluated after 60 and 120 minutes showed no differences in colour and clarity when compared to the initial time point samples. Each placebo sample appeared as clear, colourless liquid, and each antibody-containing sample appeared as slightly yellow, slightly opaque liquid. Either no particles or non-product-related particles were observed in all placebo and antibody-containing samples.

### Turbidity Assessed by Absorbance at 550 nm

The turbidity of antibody-containing and placebo formulations was evaluated by measuring the absorbance at 550 nm. The turbidity values for all antibody-containing and placebo samples showed no differences compared to the initial time point control. A550 was less than 0.07 AU at all time points for all samples.

### Size Exclusion Chromatography

The effect of pumping on the formation of soluble high and low-molecular weight species in antibody-containing samples of each formulation was evaluated by SEC, as described above. No significant changes in total percent peak areas of higher or lower molecular weight species were observed between the initial and the 120 minutes time point for samples in either formulation. Formulation F38 contained a greater fraction of high molecular weight species than those in formation F37 (2.5% vs 1.5%), and the fraction of lower weight molecular weight species did not vary significantly between samples of the two formulations (0.7% vs 0.7%). This difference in total peak area of higher molecular weight species was consistent, and observed in all samples from the pumping, filter compatibility, and syringeability studies.

### Dynamic Light Scattering

The particle distribution of antibody-containing samples was investigated by DLS, as described above, to evaluate the effect of pumping stress. Formulations were compared with respect to Z-average hydrodynamic diameter, mean polydispersity (PDI), and modality of fit. A change in the modality of fit (as evaluated based on plots of size distribution by intensity) was observed. Samples for both formulations were monomodal at TO, however, at 60 minutes, the F38 sample was multimodal and at 120 minutes both F37 and F38 samples were multimodal. Z-average diameter values were comparable for all tested samples of both formulations and showed slight upward trend as a function of stress time. A similar, but even more pronounced increasing trend was observed for PDI values obtained for samples of both tested formulations. For formulation F37 the PDI increased from 0.087 (at T0) to 0.120 and 0.136 after pumping for 60 and 120 minutes (an increase of approximately 1.4 and 1.6-fold, respectively). The obtained PDI values for F38 increased from 0.129 (at T0) to 0.238 and 0.242 after pumping for 60 and 120 minutes (an increase of approximately 1.8 and 1.9-fold, respectively). Together, the pumping stress had a slight impact on distribution of particles in F37 and F38.

MFI experiments were performed to characterise the size and concentration of particles present in antibody-containing samples over the course of the pumping study. No clear trends in subvisible particles accumulation were observed for F37 and F38 placebos over time. In contrast, progressive trends of increasing subvisible concentration were observed for F37 and F38 formulations over the duration of the pumping. The increase of subvisible particles was comparable for both formulations.

Overall, the obtained results show no increase of visible or subvisible particles in F37 and F38 subjected to pumping stress.

### Filter Compatibility Study

The filter compatibility study was designed to assess the compatibility and performance of filter materials for the final API formulations. Samples of placebo and antibody-containing formulations were passed through a 0.22 μm PES filter, a 0.22 μm PVDF filter, or kept unfiltered as a control. Analytical tests were then performed to evaluate the compatibility and performance of filtration.

### Appearance

Sample appearance was assessed by physical state, colour, clarity, and relative number of visible particles as described above. Samples filtered through a PES filter or a PVDF filter showed no difference in colour and clarity when compared to the unfiltered control samples. Each placebo sample was viewed as a clear, colourless liquid, and each antibody-containing sample was viewed as a slightly yellow, slightly opaque liquid. Either no particles or non-product-related particles were observed in all samples.

### Turbidity

Sample turbidity was assessed as described above. The turbidity values for all antibody-containing and placebo samples showed no differences compared to the initial time point control. The A550 value was less than 0.05 AU at all time points for all samples. No clear trends were observed with placebo samples in respect to time point or formulation composition.

### Size Exclusion Chromatography

The effect of filtering on the formation of soluble high and low molecular weight species on antibody-containing samples of each formulation was evaluated by SEC, as described above. No significant changes in percent peak areas of higher or lower molecular weight species were observed between the unfiltered control and the membrane-filtered samples in either formulation.

### Dynamic Light Scattering and Micro-Flow Imaging

DLS was performed as described above and results showed that filtration did not change modality of fit in either formulation, regardless of filter material, and the differences in Z-average diameter of the control samples and the filtered samples were not significant. The PDI values for formulation F37 reduced from 0.106 for the control to 0.049 and 0.050 for samples filtered by PES and PVDF, respectively. Formulation F38 PDI values reduced from 0.087 for the control sample to 0.081 and 0.063 for samples filtered by PES and PVDF, respectively.

MFI experiments were performed as described above to characterise the size, and concentration of particles present in antibody-containing samples taken from the filter compatibility study. No significant differences in cumulative particle counts were observed between filtered and control placebo samples for both formulations. Samples of antibody in both formulations F37 and F38 showed an overall decrease in cumulative particle counts of all sizes in the filtered samples as compared with the control sample. The observed reduction in particles in formulation F37 was greater in PVDF filtration than PES filtration. In contrast, the overall reduction in particles of antibody-containing samples in formulation F38 was not significantly greater for one filtration material than the other.

The overall changes in particle distribution observed in DLS and MFI analysis demonstrate that filtration of the antibody is beneficial in reducing potential aggregation during material processing as simulated by the filter compatibility study. Overall, similar reductions were observed in both formulations and for both filtration materials.

### Syringeability Study

The syringeability study was designed to simulate drug product application of final formulation, and determine the suitability of each formulation for DP application with two different sizes of Injekt Solo syringes. Samples of placebo and antibody-containing formulation were expelled with a 2 ml syringe, 10 ml syringe, or left untreated as a pre-expulsion control. To achieve this, the syringe plunger was pushed through the 2 ml syringes at a linear speed of 0.2 in/min and through the 10 ml syringes at 0.6 in/min until the plunger reached the bottom and reached the force of 30 N.

### Force

Break-loose (BF) and glide (GF) forces were measured during expulsion and used to assess application suitability. Break-loose force describes the force required to initiate movement of the plunger (the initial 0.3 mm for 2 ml syringe and 0.5 mm for 10 ml syringe). Glide force Max refers to the maximum friction force required to sustain plunger movement. The maximum force value is measured from the end of the break loose region to the end of the glide force region (26 mm for 2 ml syringe and 24 mm for 10 ml syringe) prior to the point where the force reaches 30 N). In all test conditions, the peak BF and max GF required to expel the antibody-containing or placebo samples were less than 12N, and the peak BF was less than max GF: these are indicators that both formulations in either syringe could be suitable for application as simulated in the syringeability study. Comparison of the test conditions shows that BF and GF values were greater for expulsion through a 10 ml syringe as compared to a 2 ml syringe, regardless of the formulation.

### Appearance

The appearance of both placebo and antibody-containing samples pre- and post-expulsion was assessed by colour, clarity, and relative number of visible particles. No discernible differences between the control and post-expulsion samples were observed. All placebo samples were clear, colourless liquids, and all antibody containing samples were slightly yellow, slightly opaque liquids. Either no particles or non-product-related particles were observed in all samples.

### Turbidity

Sample turbidity was assessed as described above. The absorbance at 550 nm was less than 0.05 AU at for all samples, with the exception of the control condition sample of antibody in formulation F37, which was 0.137 AU. No clear trends were observed with placebo and antibody-containing samples in respect to condition or formulation composition.

### Size Exclusion Chromatography

The effect of passing antibody containing samples through a syringe on the formation of soluble high and low molecular weight species was evaluated by SEC. No significant changes in total percent peak areas of higher or lower molecular weight species were observed between the control and the post-expulsion samples at either formulation.

### Dynamic Light Scattering

DLS was used to evaluate the particle distribution of the antibody-containing samples pre- and post-expulsion through syringes of different sizes (2 and 10 ml). Samples were compared with respect to Z-average diameter, mean polydispersity (PDI), and modality of fit. Syringe expulsion did not change modality of fit in either formulation, and there were no significant differences in Z-mean average diameter and PDI of the control samples and the syringe samples in either formulation.

### Micro-Flow Imaging

MFI experiments were performed to characterize the size, and concentration of particles present in samples. Placebo samples of F37 and F38 formulations displayed an increase in cumulative particle counts in postexpulsion samples compared to the control samples, and increases were greater with the 10 ml syringe samples compared to 2 ml syringe samples. Antibody control and 2 ml syringe samples in formulation F37 did not show a significant difference, however, 10 ml syringe samples show an overall a slight increase in cumulative particle count.

Samples of antibody in formulation F38 showed a more pronounced response to syringe application. Samples passed through the 2 ml syringe showed an increase in cumulative counts of 2.7 to 3.6-fold as compared with the control, and samples passed through the 10 ml syringe showed an increase in cumulative counts of 10 to 12-fold.

Results obtained in the syringeability study showed no impact of shear stress on antibody aggregation. An elevated concentration of subvisible particles was detected only by MFI in F38.

### Example 4: Preparation of Higher-Concentration Formulation

Tangential flow filtration (TFF) was used to concentrate an antibody formulation comprising about 116 mg/ml antibody, 20 mM histidine, 140 mM proline, 150 mM arginine and 0.02% w/v polysorbate 80 to a formulation comprising about 200 mg/ml antibody (‘High Conc. 2’). During TFF, an intermediate high concentration formulation was prepared at about 150 mg/ml (‘High Conc. 1’). The High Conc. 2 formulation was diluted to prepare a formulation comprising 100 mg/ml antibody (‘DP Test’). The original starting material was also diluted to prepare a formulation comprising 100 mg/ml antibody (‘DP Control’).

Following concentration, the concentration, density and viscosity of each formulation was assessed, as shown in Table 6. The viscosity was compared to purified antibody preparations in 20 mM histidine, 140 mM proline and 150 mM arginine, but not containing polysorbate 80.

TABLE 6
Characteristics of antibody formulations

| | | Viscosity @ | Density @ |
|---|---|---|---|
| | Ab. | 20° C. | 20° C. |
| Formulation | conc (mg/ml) | (mPa*S) | (g/cm³) |
| Purified formulation | 73.5 | 2.2 | |
| (no polysorbate 80) | 99.3 | 2.9 | |
| | 110.8 | 3.3 | |
| | 139.9 | 5.0 | |
| | 154.1 | 6.7 | |
| | 171.8 | 9.4 | |
| | 194.6 | 13.7 | |
| | 215.6 | 24.1 | |
| DP Control (pre-TFF) | 100 | 3.05 | 1.04 |
| DP Test | 114.9 | 3.81 | 1.04 |
| High Conc. 1 | 148.8 | 6.63 | 1.05 |
| High Conc. 2 | 215.4 | 26.90 | 1.07 |

The stress stability of the high concentration formulations (DP control; DP Test; High Conc. 1; and High Conc. 2) was assessed by subjecting the formulations to 35° C. for up to 5 weeks in both glass vials and pre-filled glass syringes (PFS). There was no effect on protein concentration or pH (pH 6.1) in either the syringes or vials after 5 weeks at 35° C.

Micro-Flow Imaging (MFI) was used as described above to characterise the size, concentration, and morphology of particles present in the samples. No significant differences were observed in the size, number and/or morphology of particles present between the different concentration formulations after 5 weeks at 35° C.

The % high molecular weight species and monomer was determined using SEC as described above. Formulations were assessed at time-zero and after 5 weeks held at 5° C. or 35° C. and the results are shown in Table 7.

TABLE 7
Percent monomer and high molecular weight species in high concentration formulations

| | | DP Control | | DP Test | | DP Test | | DP Test | |
|---|---|---|---|---|---|---|---|---|---|
| | | (100 mg/ml) | | (about 100 mg/ml) | | (about 150 mg/ml) | | (about 200 mg/ml) | |
| Sample | | Glass | | Glass | | Glass | | Glass | |
| Time Point | Analysis | vial | PFS | vial | PFS | vial | PFS | vial | PFS |
| 0 | % monomer | 99.2 | 99.2 | 99.1 | 99.1 | 99.1 | 99.0 | 98.9 | 98.9 |
| | % HMW | 0.6 | 0.6 | 0.7 | 0.7 | 0.8 | 0.8 | 1.0 | 1.0 |
| 5 weeks @ | % monomer | N/A | 98.5 | N/A | 98.4 | N/A | 98.2 | N/A | 97.9 |
| 5° C. | % HMW | N/A | 0.9 | N/A | 1.0 | N/A | 1.2 | N/A | 1.6 |
| 5 weeks @ | % monomer | 97.4 | 97.5 | 97.3 | 97.3 | 96.8 | 96.9 | 96.1 | 96.2 |
| 35° C. | % HMW | 1.9 | 1.8 | 2.1 | 2.1 | 2.5 | 2.5 | 3.3 | 3.2 |

As shown above, high concentration antibody formulations exhibited long term thermal stability.

### Example 5: Long-Term Stability of High Concentration Formulations in Vials

The long-term stability of the high concentration DP control formulation from Example 4 was assessed, using methodology described above, by holding the formulation at 5° C. (±3° C.) or 25° C. (±2° C.) for 36 months and 24 months, respectively, and the results are shown in Tables 8 and 9.

TABLE 8
Long-term stability of DP control formulation after 36 months at 5° C. ± 3° C.

| | | Number of months | | | | | | | |
|---|---|---|---|---|---|---|---|---|---|
| Test | Provisional limits | 0 | 3 | 6 | 9 | 12 | 18 | 24 | 36 |
| Container closure | Pass if vial is integral | Pass | NS | Pass | NS | Pass | Pass | Pass | Pass |
| integrity test | | | | | | | | | |
| Dynamic light | Z-average (no limit | 10.2 | NS | NS | NS | 10.3 | NS | 10.3 | 13.9 |
| scattering | specified) | | | | | | | | |
| | Polydispersity (no | 0.044 | NS | NS | NS | 0.043 | NS | 0.042 | 0.199 |
| | limit specified) | | | | | | | | |
| Sub-visible | ≥2 μm per container | NT | 3980 | 7528 | 27505 | 9663 | 3967 | 602 | 698 |
| particle count | ≥5 μm per container | NT | 548 | 2242 | 7918 | 3848 | 923 | 83 | 93 |
| (low volume) | ≤6,000 particles of ≥10 μm | NT | 68 | 557 | 1438 | 1215 | 170 | 5 | 12 |
| | per container | | | | | | | | |
| | ≤600 particles of ≥10 μm | NT | 2 | 33 | 87 | 93 | 13 | 0 | 0 |
| | per container | | | | | | | | |
| Description | 1°: Slightly opalescent to | Pass | Pass | Pass | Pass | Pass | Pass | Pass | Pass |
| | clear, yellow to colourless | | | | | | | | |
| | liquid. No visible particles. | | | | | | | | |
| | 2°: Slightly opalescent to | Zero | Zero | No visible | Zero | Zero | Zero | No visible | Zero |
| | celar, yellow to colourless | particles | particles | particles | particles | particles | particles | particles | particles |
| | liquid, which may contain | present | present | | observed | observed | observed | | observed |
| | particles. | | | | | | | | |
| pH | 5.8-6.4 | 6.0 | 5.9 | 6.0 | 6.0 | 6.0 | 6.0 | 6.0 | 6.0 |
| Total protein | 90-110 mg/ml | 100 | 98 | 99 | 99 | 97 | 99 | 99 | 99 |
| concentration | | | | | | | | | |
| SE-HPLC | ≤5.0% high mol. wt | 1.1 | 1.2 | 1.3 | 1.4 | 1.6 | 1.6 | 1.7 | 1.7 |
| | ≥95.0% monomer | 98.5 | 98.3 | 98.3 | 98.2 | 98.1 | 98.1 | 98.0 | 98.0 |
| | ≤5.0% fragments | 0.4 | 0.5 | 0.4 | 0.4 | 0.3 | 0.4 | 0.3 | 0.3 |
| CEX-HPLC | ≤30% acidic species | 8 | 8 | 7 | 7 | 8 | 7 | 7 | 7 |
| | ≥60% main peak | 85 | 85 | 85 | 85 | 85 | 85 | 85 | 85 |
| | ≤30% basic species | 8 | 7 | 7 | 7 | 7 | 7 | 8 | 8 |
| SDS-PAGE | ≥95% sum of heavy | 100 | 100 | 100 | 100 | 100 | 100 | 100 | 100 |
| (reduced) | chain + light chain | | | | | | | | |
| | The heavy chain band at | Pass | Pass | Pass | Pass | Pass | Pass | Pass | Pass |
| | ~54 kDa and light chain band | | | | | | | | |
| | at ~26 kDa of the test sample | | | | | | | | |
| | have a migration consistent | | | | | | | | |
| | with the ref. standard | | | | | | | | |
| SDS-PAGE | ≥85% main band | 97 | 94 | 98 | 98 | 99 | 100 | 100 | 98 |
| (non-reduced) | | | | | | | | | |
| CE-SDS | No limits specified. | NS | NS | NS | NS | NS | 98 | 98 | 98 |
| (reduced) | Report % sum heave | | | | | | | | |
| | chain and light chain. | | | | | | | | |
| CE-SES (non- | No limits specified. | NS | NS | NS | NS | NS | 98 | 99 | 98 |
| reduced) | Report % main peak. | | | | | | | | |
| | No limits specified. | NS | NS | NS | NS | NS | <2 | 1 | <2 |
| | Report % fragment. | | | | | | | | |
| Relative potency | 60-150% potency relative | 94 | 105 | 107 | 108 | 101 | 98 | 110 | 103 |
| | to reference standard | | | | | | | | |
| | Positive for FXIIa inhibition | Pass | Pass | Pass | Pass | Pass | Pass | Pass | Pass |
| Endotoxin | ≤25.00 EU/ml | <2.50 | NS | NS | NS | <2.50 | NS | <2.50 | <2.5 |
| Sterility | No microbial growth | Pass | NS | NS | NS | NS | NS | NS | NS |
| | detected | | | | | | | | |

NS: Not scheduled;
NT: Not tested;
1°: Primary limits relevant at zero time;
2°: Secondary limits relevant after time zero

TABLE 9
Long-term stability of DP control formulation after 24 months at 25° C. ± 2° C.

| | | Number of months | | | | | | |
|---|---|---|---|---|---|---|---|---|
| Test | Provisional limits | 0 | 3 | 6 | 9 | 12 | 18 | 24 |
| Container closure | Pass if vial is integral | Pass | NS | Pass | NS | Pass | Pass | Pass |
| integrity test | | | | | | | | |
| Dynamic light | Z-average (no limit specified) | 10.2 | NS | 10.4 | NS | 10.9 | 11.1 | 11.4 |
| scattering | Polydispersity (no limit | 0.044 | NS | 0.062 | NS | 0.097 | 0.128 | 0.153 |
| | specified) | | | | | | | |
| Sub-visible | ≥2 μm per container | 3980 | 6010 | 10772 | 18615 | 6787 | 1798 | 3272 |
| particle count | ≥5 μm per container | 548 | 1430 | 2180 | 9258 | 2275 | 390 | 817 |
| (low volume) | ≤6,000 particles of ≥10 μm | 68 | 318 | 297 | 4162 | 618 | 53 | 160 |
| | per container | | | | | | | |
| | ≤600 particles of ≥10 μm | 2 | 17 | 5 | 337 | 40 | 2 | 5 |
| | per container | | | | | | | |
| Description | 1°: Slightly opalescent to | Pass | Pass | Pass | Pass | Pass | Pass | Pass |
| | clear, yellow to colourless | | | | | | | |
| | liquid. No visible particles. | | | | | | | |
| | 2°: Slightly opalescent to | Zero | No visible | Zero | Zero | Zero | Zero | No visible |
| | celar, yellow to colourless | particles | particles | particles | particles | particles | particles | particles |
| | liquid, which may contain | present | observed | observed | observed | observed | observed | observed |
| | particles. | | | | | | | |
| pH | 5.8-6.4 | 5.9 | 6.0 | 6.0 | 6.0 | 6.0 | 6.0 | 6.0 |
| Total protein | 90-110 mg/ml | 98 | 98 | 99 | 97 | 99 | 98 | 99 |
| concentration | | | | | | | | |
| SE-HPLC | ≤5.0% high mol. wt | 1.2 | 1.7 | 2.0 | 2.2 | 2.2 | 2.3 | 2.4 |
| | ≥95.0% monomer | 98.3 | 97.8 | 97.6 | 97.4 | 97.3 | 97.0 | 96.9 |
| | ≤5.0% fragments | 0.5 | 0.5 | 0.4 | 0.4 | 0.5 | 0.7 | 0.7 |
| CEX-HPLC | ≤30% acidic species | 8 | 7 | 8 | 9 | 8 | 10 | 11 |
| | ≥60% main peak | 85 | 84 | 84 | 84 | 84 | 83 | 82 |
| | ≤30% basic species | 7 | 8 | 8 | 7 | 8 | 7 | 7 |
| SDS-PAGE | ≥95% sum of heavy | 100 | 100 | 100 | 99 | 100 | 98 | 100 |
| (reduced) | chain + light chain | | | | | | | |
| | The heavy chain band at | Pass | Pass | Pass | Pass | Pass | Pass | Pass |
| | ~54 kDa and light chain band | | | | | | | |
| | at ~26 kDa of the test sample | | | | | | | |
| | have a migration consistent | | | | | | | |
| | with the ref. standard | | | | | | | |
| SDS-PAGE | ≥85% main band | 94 | 96 | 91 | 94 | 93 | 88 | 85 |
| (non-reduced) | | | | | | | | |
| CE-SDS | No limits specified. | NS | NS | NS | NS | NS | 95 | 93 |
| (reduced) | Report % sum heave | | | | | | | |
| | chain and light chain. | | | | | | | |
| CE-SES | No limits specified. | NS | NS | NS | NS | NS | 89 | 88 |
| (non-reduced) | Report % main peak. | | | | | | | |
| | No limits specified. | NS | NS | NS | NS | NS | 10 | 12 |
| | Report % fragment. | | | | | | | |
| Relative potency | 60-150% potency relative | 105 | 105 | 110 | 100 | 101 | 105 | 106 |
| | to reference standard | | | | | | | |
| | Positive for FXIIa inhibition | Pass | Pass | Pass | Pass | Pass | Pass | Pass |
| Endotoxin | ≤25.00 EU/ml | <2.50 | NS | NS | NS | <2.50 | NS | <2.50 |
| Sterility | No microbial growth detected | Pass | NS | NS | NS | NS | NS | NS |

NS: Not scheduled;
1°: Primary limits relevant at zero time;
2°: Secondary limits relevant after time zero

### Example 6: Stability of High Concentration Formulations n Pre-Filled Syringes

The stability of the high concentration DP control formulation (Example 4) in pre-filled syringes was assessed at 3, 6 and 12 months, using methodology described above, by holding the formulation at 5° C. (±3° C.) or 25° C. (±2° C.). The results are shown in Tables 10 to 15.

### Table 10.1: Stability of DP Control Formulation in Pre-Filled Syringes at 5° C.±3° C.

TABLE 10.2
Stability of DP control formulation in pre-filled syringes at 5° C. ± 3° C.
DP in PFS, 1.8 ml fill

| | | +5° C. | | |
|---|---|---|---|---|
| | | t (m) | | |
| | | 0 | 3 | 6 |
| Visual Description | | Yellow with slight opalescence, No Visible Particles | | |
| | | 1.8 ml | 1.8 ml | 1.8 ml |
| Protein Concentration | | 173 | 171 | 170 |
| (mg/ml) | | | | |
| pH | | 6 | 6 | 6 |
| Potency | | 93 | 97 | 105 |
| SE-HPLC | | 1.1 | 2.0 | 2.4 |
| (% aggregates) | | | | |
| CE-SDS (% fragments) NR | | 1 | 2 | 2 |
| Acidic Variants | | 8% | 8% | 8% |
| (IEX-HPLC) | | | | |
| Basic Variants | | 6% | 8% | 7% |
| (IEX-HPLC) | | | | |
| Particles | ≥10 μm | 42 | 12 | 18 |
| Counts per ml | ≥25 μm | 2 | 0 | 1 |

| | | | |
|---|---|---|---|
| Osmolality (mOsm/Kg) | 464 | 465 | 445 |

TABLE 11.1
Stability of DP control formulation in pre-filled syringes at 25° C. ± 2° C.
DP in PFS, 1.2 ml fill

| | | +25° C. | | | | |
|---|---|---|---|---|---|---|
| | | t (m) | | | | |
| | | 0 | 3 | 6 | 9 | 12 |
| Visual Description | | Yellow with slight opalescence, No Visible Particles | | | | |
| | | 1.2 ml | 1.2 ml | 1.2 ml | 1.2 ml | 1.2 ml |
| Protein Concentration | | 172 | 170 | 170 | 170 | 178 |
| (mg/ml) | | | | | | |
| pH | | 5.9 | 6.0 | 6.0 | 6.0 | 6.0 |
| Potency | | 101 | 91 | 104 | 103 | 105 |
| SE-HPLC | | 1.1 | 2.9 | 3.3 | 3.4 | 3.6 |
| (% aggregates) | | | | | | |
| CE-SDS (% fragments) NR | | 1 | 3 | 4 | 6 | 6 |
| Acidic Variants | | 8% | 8% | 8% | 9% | 9% |
| (IEX-HPLC) | | | | | | |
| Basic Variants | | 6% | 7% | 8% | 9% | 8% |
| (IEX-HPLC) | | | | | | |
| Particles | ≥10 μm | 21 | 69 | 358 | 540 | 1080 |
| Counts per ml | ≥25 μm | 1 | 3 | 18 | 25 | 49 |

| | | | | | |
|---|---|---|---|---|---|
| Osmolality (mOsm/Kg) | 467 | 477 | 447 | NS | NS |

TABLE 11.2
Stability of DP control formulation in pre-filled syringes at 25° C. ± 2° C.
DP in PFS, 1.8 ml fill

| | | +25° C. | | |
|---|---|---|---|---|
| | | t (m) | | |
| | | 0 | 3 | 6 |
| Visual Description | | Yellow with slight opalescence, No Visible Particles | | |
| | | 1.8 ml | 1.8 ml | 1.8 ml |
| Protein Concentration | | 173 | 170 | 169 |
| (mg/ml) | | | | |
| pH | | 6 | 6 | 6 |
| Potency | | 93 | 91 | 116 |
| SE-HPLC | | 1.1 | 2.9 | 3.3 |
| (% aggregates) | | | | |
| CE-SDS (% fragments) NR | | 1 | 3 | 4 |
| Acidic Variants | | 8% | 8% | 8% |
| (IEX-HPLC) | | | | |
| Basic Variants | | 6% | 7% | 8% |
| (IEX-HPLC) | | | | |
| Particles | ≥10 μm | 42 | 49 | 144 |
| Counts per ml | ≥25 μm | 2 | 0 | 5 |

| | | | |
|---|---|---|---|
| Osmolality (mOsm/Kg) | 464 | 457 | 450 |

TABLE 12
Stability of DP control formulation in pre-filled syringes after 12 months at 5° C. ± 2° C.
DP in PFS (2.2 ml) + 5° C.

| | | t (m) | | | | | | |
|---|---|---|---|---|---|---|---|---|
| | | 0 | 3 | 6 | 9 | 12 | 18 | 24 |
| Visual Description | | Pass, No | Pass, No | Pass, No | Pass, No | Pass, No | Pass, No | Pass, No |
| | | Visible | Visible | Visible | Visible | Visible | Visible | Visible |
| | | Particles | Particles | Particles | Particles | Particles | Particles | Particles |
| Protein Concentration | | 170 | 172 | 167 | 167 | 171 | 171 | 169 |
| (mg/ml) | | | | | | | | |
| pH | | 6.0 | 6.0 | 6.0 | 6.0 | 6.0 | 6.0 | 6.0 |
| Potency | | 95 | 95 | 105 | 103 | 97 | 108 | 99 |
| SE-HPLC | | 1.7 | 2.3 | 2.8 | 3.0 | 3.1 | 3.2 | 3.4 |
| (% aggregates) | | | | | | | | |
| CE-SDS | | 1 | 1 | 2 | 2 | 2 | 1 | 2 |
| (% fragments) NR | | | | | | | | |
| Acidic Variants | | 10 | 9 | 10 | 9 | 9 | 9 | 9 |
| (IEX-HPLC) | | | | | | | | |
| Basic Variants | | 7 | 8 | 8 | 8 | 8 | 8 | 9 |
| (IEX-HPLC) | | | | | | | | |
| Particle Counts | ≥10 μm | 581 | 380 | 208 | 331 | 227 | 364 | 966 |
| per container | | | | | | | | |
| Particle Counts | ≥25 μm | 40 | 106 | 18 | 25 | 6 | 32 | 129 |
| per container | | | | | | | | |

| | | | | | | | |
|---|---|---|---|---|---|---|---|
| Osmolality (mOsm/Kg) | 453 | NS | 462 | NS | 458 | NS | NS |

TABLE 13
Stability of DP control formulation in pre-filled syringes after 12 months at 25° C. ± 2° C.
DP in PFS (2.2 ml) + 25° C.

| | | t (m) | | | | | | |
|---|---|---|---|---|---|---|---|---|
| | | 0 | 3 | 6 | 9 | 12 | 18 | 24 |
| Visual Description | | Pass, No | Pass, No | Pass, No | 1 to 2 | 10-20 | >10 | >30 |
| | | Visible | Visible | Visible | visible | visible | visible | visible |
| | | Particles | Particles | Particles | particles | particles | particles | particles |
| Protein Concentration | | 170 | 170 | 169 | 172 | 168 | 169 | 171 |
| (mg/ml) | | | | | | | | |
| pH | | 6.0 | 6.0 | 6.0 | 6.0 | 6.0 | 5.9 | 5.9 |
| Potency | | 95 | 87 | 105 | 111 | 102 | 113 | 107 |
| SE-HPLC | | 1.7 | 3.2 | 4.0 | 4.3 | 4.5 | 4.8 | 5.3 |
| (% aggregates) | | | | | | | | |
| CE-SDS | | 1 | 3 | 4 | 5 | 7 | 9 | 12 |
| (% fragments) NR | | | | | | | | |
| Acidic Variants | | 10 | 9 | 9 | 10 | 11 | 12 | 13 |
| (IEX-HPLC) | | | | | | | | |
| Basic Variants | | 7 | 9 | 9 | 9 | 9 | 9 | 10 |
| (IEX-HPLC) | | | | | | | | |
| Particle Counts | ≥10 μm | 581 | 315 | 991 | 3585 | 1120 | 2682 | 2465 |
| per container | | | | | | | | |
| Particle Counts | ≥25 μm | 40 | 33 | 126 | 606 | 137 | 449 | 320 |
| per container | | | | | | | | |

| | | | | | | | |
|---|---|---|---|---|---|---|---|
| Osmolality (mOsm/Kg) | 453 | NS | 461 | NS | 426 | NS | NS |

TABLE 14
Stability of DP control formulation in hand-filled
syringes after 12 months at 5° C. ± 2° C.
DP in PFS (2.2 ml) + 5° C. (Hand filled)

| | | t (m) | | | | |
|---|---|---|---|---|---|---|
| | | 0 | 3 | 6 | 9 | 12 |
| Visual Description | | Pass, | Pass, | Pass, | Pass, | Pass, |
| | | No Visible | No Visible | No Visible | No Visible | No Visible |
| | | Particles | Particles | Particles | Particles | Particles |
| Protein Concentration | | 170 | 172 | 167 | 169 | 171 |
| (mg/ml) | | | | | | |
| pH | | 6.0 | 6.0 | 6.0 | 6.0 | 6.0 |
| Potency | | 95 | 95 | 105 | 103 | 97 |
| SE-HPLC | | 1.7 | 2.3 | 2.8 | 3.0 | 3.1 |
| (% aggregates) | | | | | | |
| CE-SDS | | 1 | 1 | 2 | 2 | 2 |
| (% fragments) NR | | | | | | |
| Acidic Variants | | 10 | 9 | 10 | 9 | 9 |
| (IEX-HPLC) | | | | | | |
| Basic Variants | | 7 | 8 | 8 | 8 | 8 |
| (IEX-HPLC) | | | | | | |
| Particle Counts | ≥10 μm | 581 | 380 | 208 | 331 | 227 |
| per container | | | | | | |
| Particle Counts | ≥25 μm | 40 | 106 | 18 | 25 | 6 |
| per container | | | | | | |

| | | | | | |
|---|---|---|---|---|---|
| Osmolality (mOsm/Kg) | 453 | NS | 462 | NS | 458 |

TABLE 15
Stability of DP control formulation in hand-filled
syringes after 12 months at 25° C. ± 2° C.
DP in PFS (2.2 ml) + 25° C.

| | | t (m) | | | | |
|---|---|---|---|---|---|---|
| | | 0 | 3 | 6 | 9 | 12 |
| Visual Description | | Pass, | Pass, | Pass, | 1 to 2 | 10-20 |
| | | No Visible | No Visible | No Visible | visible | visible |
| | | Particles | Particles | Particles | particles | particles. |
| Protein Concentration | | 170 | 170 | 169 | 172 | 168 |
| (mg/ml) | | | | | | |
| pH | | 6.0 | 6.0 | 6.0 | 6.0 | 6.0 |
| Potency | | 95 | 87 | 105 | 111 | 102 |
| SE-HPLC | | 1.7 | 3.2 | 4.0 | 4.3 | 4.5 |
| (% aggregates) | | | | | | |
| CE-SDS | | 1 | 3 | 4 | 5 | 7 |
| (% fragments) NR | | | | | | |
| Acidic Variants | | 10 | 9 | 9 | 10 | 11 |
| (IEX-HPLC) | | | | | | |
| Basic Variants | | 7 | 9 | 9 | 9 | 9 |
| (IEX-HPLC) | | | | | | |
| Particle Counts | ≥10 μm | 581 | 315 | 991 | 3585 | 1120 |
| per container | | | | | | |
| Particle Counts | ≥25 μm | 40 | 33 | 126 | 606 | 137 |
| per container | | | | | | |

| | | | | | |
|---|---|---|---|---|---|
| Osmolality (mOsm/Kg) | 453 | NS | 461 | NS | 426 |

### Example 7: Exemplary Formulations

Two stable high concentration formulations based on the data presented herein are shown in Table 16. The acceptance criteria for these formulations are shown in Table 17.

TABLE 16
Exemplary high concentration formulations

| | Amount or | | Amount or | |
|---|---|---|---|---|
| Excipient | concentration | | concentration | |
| anti-FXII antibody | 100 | mg/ml | 170 | mg/ml |
| L-Histidine | 20 | mM | 20 | mM |
| L-Arginine | 150 | mM | 150 | mM |
| Monohydrochloride | | | | |
| L-Proline | 140 | mM | 140 | mM |
| Polysorbate 80 | 0.02% | w/v | 0.02% | w/v |
| pH | 6.1 | | 6.1 | |
| Osmolality | ~430 | mOsm/kg | ~450 | mOsm/kg |
| (mOsm/kg) | | | | |
| Viscosity | 2.8 | cP (@ 25° C.); | 7.5 | cP (@ 25° C.); |
| | 3.3 | cP (@ 20° C.) | 8.9 | cP (@ 20° C.) |

TABLE 17
Acceptance criteria for exemplary high concentration formulations

| Parameter | Test | Acceptance criteria | |
|---|---|---|---|
| Protein content | UV Spectrophotometry (A280 nm) | 160-180 | mg/ml |
| Purity | Size Exclusion HPLC | ≤4.5% | HMWS |
| | | ≥95.0% | monomer |
| | | ≤20% | acidic species |
| | | ≥60% | main peak |
| | Cation Exchange (CEX) HPLC | ≤20% | basic species |
| | CE-SDS (non-reducing) | ≥90% | main peak |
| | | ≤8% | LMWS |
| | CE-SDS (reducing) | ≥95% | sum of HC + LC |
| Excipients | Polysorbate 80 | 0.010%-0.030% | (w/v) |
| | Histidine | 12-15 | mM |
| | Arginine | 100-160 | mM |
| | Proline | 90-150 | mM |
| Purity/Safety | Sterility | No growth of micro-organisms | |
| | Endotoxin | ≤25.00 | EU/ml |
| Quality | Visible Particles | None | |
| | Clarity | ≤25 | NTU |
| | Colour | Between BY2 and BY5 | |
| | Osmolality | 430-530 | mOsm/kg |
| | pH | 5.8-6.4 | |
| | Sub-visible Particles | ≤6000 particles of ≥10 μm per | |
| | | container | |
| | | ≤600 particles of ≥25 μm per | |
| | | container | |
| | Extractable volume | ≥1.2 | ml |

### Example 8: Single-Dose Pharmacokinetic Study of High-Concentration Formulations Following Subcutaneous Administration to Rabbits

The objective of this study was to assess the pharmacokinetic (PK) properties of two high concentration formulations (100 and 170 mg/ml as shown in Example 7) after a single subcutaneous administration to rabbits.

The blood samples of the New Zealand White Rabbits were processed for plasma and analysed. The analysis of the plasma samples showed a similar increase of drug concentration profile for both groups of anti-FXII antibody concentration. The levels of the anti-FXII antibody antigen in the rabbit plasma samples were determined by using a validated assay (ELISA). Maximum plasma concentrations (Cₘₐ) of the anti-FXII antibody were generally observed at 24 or 48 hours post dosing. Cₘₐₓ and area under the curve (AUC) for group 1 and group 2, increased approximately to the same extent on test day 1. The mean terminal plasma elimination half-life ty of the anti-FXII antibody was comparable across all treatments. No noteworthy gender differences were noted for any pharmacokinetic parameter. The marginal differences noted between the various treatments tested for a few pharmacokinetic parameters are all considered to be within the normal variability for this sample size (three animals per sex and time-point). The results of the pharmacokinetic evaluation of CSL312 in rabbit serum are summarized in Table 18.

TABLE 18
Pharmacokinetic analysis of DP (means ± standard deviation)

| | | | | | | | | | |
|---|---|---|---|---|---|---|---|---|---|
| Drug Conc. | Dose^{#1} | Animal | Cₘₐₓ | tₘₐₓ | t_{1/2} | Kₑₗ | AUC_{0-t last} | AUC_{0-inf} | AUC_{0-t last}/dose |
| [mg/ml] | [mg/kg] | No. | [μg/ml] | [h] | [h] | [1/h] | [μg · h/ml] | [μg · h/ml] | [g · h/l] |

Males

| | | | | | | | | | |
|---|---|---|---|---|---|---|---|---|---|
| 100 | 20 | 1 | 67.53 | 48 | 84.35 | 0.0082 | 7953.38 | 11055.78 | 397.8 |
| | | 2 | 52.26 | 24 | 117.05 | 0.0059 | 5550.47 | 9137.87 | 277.5 |
| | | 3 | 70.59 | 48 | 49.47 | 0.0140 | 7012.31 | 7990.66 | 350.6 |
| | | Mean | 63.46 ± | 40.0 ± | 83.26 ± | 0.0094 ± | 6839.72 ± | 10096.83 ± | 342.0 ± |
| | | | 9.8 | 13.9 | 47.79 | 0.0042 | 1212.2 | 1356.2 | 60.6 |
| 170 | 20 | 7 | 73.73 | 48 | 114.94 | 0.0060 | 9016.31 | 15057.97 | 450.8 |
| | | 8 | 70.72 | 24 | 88.03 | 0.0079 | 8706.48 | 12400.41 | 435.3 |
| | | 9 | 82.84 | 48 | 110.35 | 0.0063 | 9947.69 | 16155.04 | 497.4 |
| | | Mean | 75.76 ± | 40.0 ± | 104.44 ± | 0.0067 ± | 9223.49 ± | 14537.81 ± | 461.2 ± |
| | | | 6.3 | 13.9 | 14.40 | 0.0010 | 6646.0 | 1930.6 | 32.3 |

Females

| | | | | | | | | | |
|---|---|---|---|---|---|---|---|---|---|
| 100 | 20 | 4 | 76.41 | 48 | 93.19 | 0.0074 | 9383.27 | 13722.02 | 469.2 |
| | | 5 | 73.31 | 24 | 90.71 | 0.0076 | 8810.06 | 12656.57 | 440.5 |
| | | 6 | 79.74 | 48 | 91.75 | 0.0076 | 9943.87 | 14478.75 | 497.2 |
| | | Mean | 76.49 ± | 40.0 ± | 91.88 ± | 0.0075 ± | 9379.07 ± | 13619.11 ± | 469.0 ± |
| | | | 3.2 | 13.9 | 1.25 | 0.0001 | 566.9 | 915.4 | 28.4 |
| 170 | 20 | 10 | 103.47 | 24 | 137.39 | 0.0050 | 12433.46 | 22743.18 | 621.7 |
| | | 11 | 73.74 | 48 | 116.38 | 0.0060 | 9411.46 | 15820.82 | 470.6 |
| | | 12 | 63.31 | 48 | 66.03 | 0.0105 | 63.70.69 | 8125.82 | 318.5 |
| | | Mean | 80.17 ± | 40.0 ± | 106.60 ± | 0.0072 ± | 9405.28 ± | 15563.27 ± | 470.3 ± |
| | | | 20.8 | 13.9 | 36.67 | 0.0029 | 3031.39 | 7312.1 | 151.6 |

^{#1}Values obtained from serum analysis, all other values calculated by pharmacokinetic analysis.

Overall, both high concentration formulations showed a similar PK profile following SC administration.

### Example 9: A Repeated-Dose Local Tolerance of High Concentration Formulations Following Subcutaneous Administration in Rabbits

The local tolerance of two high-concentration formulations (100 and 170 mg/mL as shown in Example 7) after subcutaneous administration to rabbits on two separate days, 1 week apart, was assessed. The New Zealand White Rabbits were macroscopically inspected for subcutaneous injections under the dorsal skin at 1, 2, 6, 24, 48 and 72 hours after administration. Following an overall observation period (11 days after the first administration), all animals were sacrificed and injection sites were examined macro- and microscopically.

There were no signs of toxicity in any rabbit during the observation period and no effect on body weight observed. None of the animals died prematurely. No local irritation at any application site was apparent at any time during the study period following administration with the two high-concentration formulations. Necropsy did not reveal any changes, either. The histopathological examination of the rabbit skin treated subcutaneously with the two formulations revealed no drug related morphological changes following subcutaneous administration. There were only few mild to moderate morphological changes observed for the drug-treated skin on the right side and the negative control treated with 0.9% NaCl solution on the left side. All these changes are considered to be procedure-related caused by the technical application and not to be drug-related.

Overall, subcutaneous administration with two high-concentration formulations administered on two days was well tolerated and revealed no drug-related changes at the injection sites.

1. A liquid pharmaceutical formulation comprising at least 100 mg/ml of a protein comprising an antigen binding domain that binds to or specifically binds to Factor XII and/or an activated form thereof, an organic acid buffer, a non-ionic surfactant and an amino acid stabiliser, wherein the formulation has a pH of 5.0 to 6.5 and a viscosity of less than 30 mPa*s at 20° C.

2. The formulation according to claim 1, wherein the protein is present in the formulation at a concentration of at least 150 mg/ml.

3. The formulation according to claim 1, wherein the protein is present in the formulation at a concentration of 160 mg/ml to 180 mg/ml.

4. The formulation according to claim 1, wherein the formulation is an aqueous formulation.

5. The formulation according to claim 1, wherein the organic acid buffer is selected from the group consisting of a histidine buffer and a glutamate buffer.

6. The formulation according to claim 1, wherein the organic acid buffer is a histidine buffer.

7. The formulation according to claim 1, wherein the non-ionic surfactant is selected from the group consisting of polysorbate 80, polysorbate 20, and poloxamer 188.

8. The formulation according to claim 1, wherein the non-ionic surfactant is polysorbate 80.

9. The formulation according to claim 1, wherein the amino acid stabiliser is selected from the group consisting of proline, arginine, salts thereof, and a combination thereof.

10. The formulation according to claim 1, wherein the amino acid stabiliser is proline.

11. The formulation according to claim 1, wherein the formulation further comprises a polyol.

12. The formulation according to claim 1, wherein the formulation comprises a histidine buffer, proline, and polysorbate 80.

13. The formulation according to claim 12, wherein the formulation further comprises arginine monohydrochloride.

14. The formulation according to claim 1, wherein the formulation comprises 100 mg/ml to 110 mg/ml of the protein, a histidine buffer, polysorbate 80 and proline and arginine monohydrochloride as stabilisers, wherein the formulation has a pH of 5.5 to 6.5 and a viscosity of less than 10 mPa*s at 20° C.

15. The formulation according to claim 1, wherein the formulation comprises 160 mg/ml to 180 mg/ml of the protein, a histidine buffer, polysorbate 80 and proline and arginine monohydrochloride as stabilisers, wherein the formulation has a pH of 5.5 to 6.5 and a viscosity of less than about 10 mPa*s at 20° C.

16. The formulation according to claim 1, wherein the formulation has a pH of 5.8 to 6.4 and comprises 12 mM to 25 mM L-histidine buffer, 0.01% to 0.03% (w/v) polysorbate 80, 90 mM to 150 mM L-proline, and 100 mM to 160 mM L-arginine monohydrochloride.

17. The formulation according to claim 1, wherein the formulation has a pH of 5.8 to 6.4 and comprises 20 mM L-histidine buffer, 0.02% (w/v) polysorbate 80, 140 mM L-proline, and 150 mM L-arginine monohydrochloride.

18. The formulation according to claim 1, wherein the viscosity of the formulation is less than 9 mPa*s at 20° C.

19. The formulation according to claim 1, wherein the formulation has a density of 1.00 to 1.10 g/cm³ at 20° C.

20. The formulation according to claim 1, wherein the formulation comprises less than 10% total aggregates of the protein.

21. The formulation according to claim 1, wherein at least 90% of the protein in the formulation is a monomer.

22. The formulation according to claim 1, wherein the antigen binding domain binds to or specifically binds to Factor XII and/or an activated form thereof and antagonises activity of the Factor XII and/or an activated form thereof and/or antagonises activation of the Factor XII and/or an activated form thereof.

23. The formulation according to claim 1, wherein the protein comprises an antigen binding domain of an antibody.

24. The formulation according to claim 1, wherein the protein is selected from the group consisting of:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)2;
(viii) a Fv;
(ix) one of (i) to (viii) is linked to a constant region of an antibody, Fc or a heavy chain constant domain (C_{H}) C_{H}2 and/or C_{H}3; or
(x) an antibody.

25. The formulation according to claim 1, wherein the protein comprises:
(i) a V_{H} comprising an amino acid sequence set forth in SEQ ID NO: 1 and a V_{L} comprising an amino acid sequence set forth in SEQ ID NO: 2
(ii) a V_{H} comprising an amino acid sequence set forth in SEQ ID NO: 3 and a V_{L} comprising an amino acid sequence set forth in SEQ ID NO: 4; or
(iii) a V_{H} comprising an amino acid sequence set forth in SEQ ID NO: 5 and a V_{L} comprising an amino acid sequence set forth in SEQ ID NO: 6.

26. The formulation according to claim 1, wherein the protein comprises:
(i) a V_{H} comprising:
(a) a CDR1 comprising a sequence set forth in SEQ ID NO: 7; a CDR2 comprising a sequence set forth in SEQ ID NO: 8; and a CDR3 comprising a sequence set forth in SEQ ID NO: 9; or
(b) a CDR1 comprising a sequence set forth in SEQ ID NO: 7; a CDR2 comprising a sequence set forth in SEQ ID NO: 10; and a CDR3 comprising a sequence set forth in SEQ ID NO: 11; or
(c) a CDR1 comprising a sequence set forth in SEQ ID NO: 7; a CDR2 comprising a sequence set forth in SEQ ID NO: 10; and a CDR3 comprising a sequence set forth in SEQ ID NO: 9; or
(d) a CDR1 comprising a sequence set forth in SEQ ID NO: 7; a CDR2 comprising a sequence set forth in SEQ ID NO: 16; and a CDR3 comprising a sequence set forth in SEQ ID NO: 9; and/or
(ii) a V_{L} comprising:
(a) a CDR1 comprising a sequence set forth in SEQ ID NO: 12; a CDR2 comprising a sequence set forth in SEQ ID NO: 13; and a CDR3 comprising a sequence set forth in SEQ ID NO: 14; or
(b) a CDR1 comprising a sequence set forth in SEQ ID NO: 12; a CDR2 comprising a sequence set forth in SEQ ID NO: 13; and a CDR3 comprising a sequence set forth in SEQ ID NO: 15.

27. The formulation according to claim 26, wherein the protein comprises a V_{H} comprising a CDR2 set forth in SEQ ID NO: 10 wherein the X at position 3 is D, the X at position 4 is I, the X at position 5 is P, the X at position 6 is T, the X at position 7 is K, and the X at position 8 is G.

28. The formulation according to claim 1, wherein the protein comprises an IgG₄ constant region.

29. The formulation according to claim 28, wherein the IgG₄ constant region is a stabilized IgG₄ constant region.

30. The formulation according to claim 1, wherein the formulation comprises 100 mg/ml and 170 mg/ml of the protein, a histidine buffer, polysorbate 80 and proline and arginine monohydrochloride as stabilisers, wherein the formulation has a pH of 5.5 to 6.5 and a viscosity of less than 30 mPa*s at 20° C. and wherein the protein comprises a V_{H} comprising an amino acid sequence set forth in SEQ ID NO: 5 and a V_{L} comprising an amino acid sequence set forth in SEQ ID NO: 6.

31. The formulation according to claim 1, wherein the formulation comprises 100 mg/ml and 170 mg/ml of the protein, a histidine buffer, polysorbate 80 and proline and arginine monohydrochloride as stabilisers, wherein the formulation has a pH of 5.5 to 6.5 and a viscosity of less than 30 mPa*s at 20° C. and wherein the protein comprises:
(i) a V_{H} comprising a CDR1 comprising a sequence set forth in SEQ ID NO: 7; a CDR2 comprising a sequence set forth in SEQ ID NO: 16; and a CDR3 comprising a sequence set forth in SEQ ID NO: 9; and
(ii) a V_{L} comprising a CDR1 comprising a sequence set forth in SEQ ID NO: 12; a CDR2 comprising a sequence set forth in SEQ ID NO: 13; and a CDR3 comprising a sequence set forth in SEQ ID NO: 14.

32.-44. (canceled)

45. A prefilled syringe comprising the formulation according to claim 1.

46. An autoinjector device comprising the formulation according to claim 1.

47. A method of antagonizing activity and/or antagonising activation of Factor XII and/or an activated form thereof in a subject in need thereof, comprising administering to the subject a therapeutically effective amount of the formulation of claim 1.

48. The method of claim 45, wherein the subject has a disease or condition selected from the group consisting of a thrombotic disorder, an inflammatory disorder, and a thrombo-inflammatory disorder.

49. The method of claim 46, wherein the disease or condition is selected from the group consisting of venous, arterial, or capillary thrombus formation; thrombus formation in the heart; thromboembolism; thrombus formation during and/or after contacting blood of a human or animal subject with artificial surfaces; disseminated intravascular coagulation (DIC); atrial fibrillation; acute coronary syndromes (ACS); atherosclerotic disease; ischaemic stroke with reperfusion; a disease associated with ischemia-reperfusion injury (IRI); neurotraumatic disorder; a neurological inflammatory disease; an interstitial lung disease; pneumonia; fibrinolysis; a disease related to FXII/FXIIa-induced kinin formation; sepsis; a disease related to FXII/FXIIa-mediated complement activation; acute respiratory distress syndrome (ARDS); organ and cell transplantation; sickle cell disease; and a condition associated with increased vascular permeability.
